(19) United States
(12) Patent Application Publication
(10) Pub. No.: US 2009/0258420 A1
(43) Pub. Date: Oct. 15, 2009

(54) Altered polypeptides, immunoconjugates thereof, and methods related thereto

(76) Inventors: Herman van Vlijmen, Mechelen (BE); Alexey Alexandrovich Lugovskoy, Woburn, MA (US); Kathryn Strauch, Bedford, MA (US); Frederick R. Taylor, Milton, MA (US)

Correspondence Address: LAHIVE & COCKFIELD, LLP / BIOGEN IDEC;FLOOR 30, SUITE 3000, ONE POST OFFICE SQUARE, BOSTON, MA, 02109-2127, US

(21) Appl. No.: 12/012,224

(22) Filed: Jan. 30, 2008

Related U.S. Application Data
(63) Continuation of application No. PCT/US06/30234, filed on Aug. 1, 2006.
(60) Provisional application No. 60/704,603, filed on Aug. 1, 2005.

(51) Int. Cl. C12N 5/10; C12N 15/63; C07K 14/00; C07K 16/00; C07H 21/00

(52) U.S. Cl. 435/358; 435/320.1; 530/350; 530/387.1; 536/23.1; 536/23.53

(57) ABSTRACT

The present invention features inter alia altered binding polypeptides having engineered cysteine residues or analogs thereof at a predetermined site within, for example, a constant region domain or a portion thereof. The engineered cysteine residues or analogs thereof provide sites for conjugating effector moieties (e.g. diagnostic or therapeutic agents) that impart novel functionality to the binding polypeptide, preferably without interfering with a desirable property (e.g. an Fc-mediated effector function). The invention includes methods for the rational design of such altered polypeptides, as well as methods for modifying (ie. conjugating) the altered polypeptides with desirable effector moieties. Particular modified binding polypeptides (ie. immunoconjugates) of altered binding polypeptides and methods for utilizing such modified binding polypeptides as protein-based therapeutics are also provided.

This application is a continuation application of International Patent Application No. PCT/US2006/30234, filed Aug. 1, 2006, which claims the benefit of priority to U.S. provisional patent application No. 60/704,603, filed on Aug. 1, 2005, the content of which is hereby incorporated by reference in its entirety.

This application is also related to U.S. Provisional Application Ser. Nos. 60/592,886 and 60/592,787, both entitled “Binding Molecules and Methods of Use Thereof,” and both filed on Jul. 30, 2005. This application is also related to PCT/US05/27262, titled “Binding Molecules and Methods of Use Thereof,” filed on Aug. 1, 2005. The contents of these applications are incorporated in their entirety by this reference.

### BACKGROUND OF THE INVENTION

Conjugation is a useful method for engineering novel functional properties in polypeptides. Conventional conjugation methods for modifying polypeptides with functional moieties, however, have certain shortcomings. First, the chemistry of the cross-linking reaction is usually very inefficient. Typically the yields of chemical cross-linking reactions are only about 10% to 20%. As a result, a significant amount of unmodified polypeptide is lost during the manufacturing. This becomes extremely important when one considers the manufacturing costs for certain low-yield proteins such as therapeutic and diagnostic antibodies. Second, the modified polypeptide produced by chemical cross-linking contains a chemical cross-linker fragment that can be immunogenic or toxic. This is especially important in therapeutic applications where the modified polypeptide may have to be administered many times. Third, many cross-linking methods are neither site-specific nor stereo-specific, and consequently, may decrease the functionality, specificity, affinity, or avidity of the polypeptide. Fourth, the cross-linking method may not allow for precise stoichiometric control of the number of modifications to the polypeptide.

Site-directed conjugation is a preferred method for polypeptide conjugation. A variety of site-specific techniques have been employed with varying degrees of specificity. In one method, native chemical groups present in a polypeptide have been modified with exogenous reagents (eg. the amine or hydroxyl groups present in lysine or tyrosine residues), but such modification methods have been shown to be essentially random, such that polypeptides are modified at multiple different sites. A heterogeneous mixture of polypeptides so modified is inadequate for diagnostic and therapeutic use as it is inadequately defined and consists of a population of different chemical entities with varying biological properties. Another approach has employed the modification of amino acid residues such as cysteines which are present relatively infrequently within a polypeptide sequence. For example, native, disulfide-bonded, cysteines may be reduced to generate a native free cysteine which can then subsequently be modified. However, these methods are also problematic, as disulfide bonds formed by native cysteines are usually essential for maintaining the structure and function of the polypeptide.

Improvements in the specificity of site-directed polypeptide conjugation have been afforded by introducing a small number of mutations within the polypeptide (e.g. a non-native amino acid residue) to thereby produce an altered polypeptide which is capable of being site-specifically modified to enable the production of a homogeneous mixture of altered binding proteins. One preferred site-directed alteration is to introduce (e.g. by site-directed mutagenesis) an engineered free cysteine into a polypeptide which can be subsequently modified by conjugation with an effector moiety. The vast majority of polypeptides do not contain free cysteine residues, and so when engineered as an alteration within a polypeptide, an engineered free cysteine presents a unique site for highly specific site-directed conjugation.

When engineered into a polypeptide, however, it is vitally important that the engineered free cysteine does not interfere with the desired function that is intrinsic to the polypeptide. An important class of polypeptides for which conjugation of functional moieties is desirable includes binding polypeptides (e.g. antibodies and other binding polypeptides) having a constant region or portion thereof derived from an immunoglobulin molecule. Attachment of effector moieties to engineered cysteine residues can improve their therapeutic and diagnostic utility. Unfortunately, however, the engineered cysteine residue will often result in drastic effects on the favorable activities (e.g. effector function) that constant region sequences provide.

Constant regions (e.g. Fc region) of immunoglobulin chains mediate effector functions that have been divided into two categories. In the first are the functions that occur independently of antigen binding; these functions confer persistence in the circulation and the ability to be transferred across cellular barriers by transcytosis (see Ward and Ghetie, Therapeutic Immunology 2:77-94, 1995). The circulatory half-life of the immunoglobulin is regulated by the affinity of the Fc region of the heavy chain for an Fc-binding protein termed the neonatal Fc receptor or FcRn (see Ghetic et al., Nature Biotechnol. 15:637-640, 1997; Kim et. al., Eur. J. Immunol. 24:542-548, 1994; Della'Acqua et al. (J. Immunol. 169:5171-5180, 2002). The second general category of effector functions include those that operate after an immunoglobulin binds an antigen; these functions involve the participation of the complement cascade or Fc gamma receptor (FcγR)-bearing cells. Binding of the Fc portion of an immunoglobulin to an FcγR causes certain immune effects, for example, endocytosis of immune complexes, engulfment and destruction of immunoglobulin-coated particles or microorganisms (also called antibody-dependent phagocytosis, or ADCP), clearance of immune complexes, lysis of immunoglobulin-coated target cells by killer cells (called antibody-dependent cell-mediated cytotoxicity, or ADCC), release of inflammatory mediators, regulation of immune system cell activation, and regulation of immunoglobulin production.

Accordingly, there is a need in the art for altered polypeptides comprising at least one constant region domain that have been rationally designed for site-directed conjugation so that the subsequently modified polypeptide retains desired Fc effector function(s). Similarly, there is a need for methods that would allow for prediction of changes in amino acid sequence which will not alter the effector functions of the polypeptide comprising a constant region domain (thus obviating the need to rely on laborious trail-and-error processes. Such therapeutics and methods or making them would be of great benefit.

### SUMMARY OF THE INVENTION

The present invention features inter alia altered binding polypeptides having engineered cysteine residues or analogs thereof within, for example, a constant region domain or a portion thereof. The engineered cysteine residues or analogs thereof provide sites for conjugating effector moieties (e.g. diagnostic or therapeutic agents) that impart novel functionality to the binding polypeptide, preferably without interfering with a desirable property (e.g. antigen-dependent effector functions (e.g. ADCC) or antigen-independent effector functions (e.g. serum half-life)) that is mediated by the constant domain. The invention includes methods for the rational design of such altered polypeptides, as well as methods for modifying (ie. conjugating) the altered polypeptides with desirable effector moieties. Particular modified binding polypeptides (ie. immunoconjugates) of altered binding polypeptides and methods for utilizing such modified binding polypeptides as protein-based therapeutics are also provided.

In one aspect, the invention pertains to an altered binding polypeptide comprising a first CH3 domain or portion thereof which comprises an engineered cysteine or thiol-containing analog thereof at one or more amino acid positions selected from the group consisting of amino acid positions 350, 352, 353, 355, 358-366, 368, 370, 371, 389-391, 394-396, 398, 400-407, 409-423 441, 443, 445, 446, and 446b, according to the EU numbering index.

In another aspect, the invention pertains to a monospecific altered binding protein comprising an altered binding polypeptide comprising an engineered cysteine or thiol-containing analog thereof at one or more amino acid positions selected from the group consisting of amino acid positions 341-441, 443, 445, and 446b, according to the EU numbering index.

In another aspect, the invention pertains to an altered binding polypeptide comprising a first CH3 domain or portion thereof, wherein the CH3 domain comprises an engineered cysteine or thiol-containing analog thereof at one or more amino acid positions selected from the group consisting of 350, 355, 361, 389, 415, 441, 443, and 446b, according to the EU numbering index.

In yet another aspect, the invention pertains to an altered binding polypeptide wherein said altered binding polypeptide is an altered antibody variant chain comprising a first CH3 domain or portion thereof which comprises an engineered cysteine or thiol-containing analog thereof at one or more amino acid positions selected from the group consisting of 341-441, 443, 445, and 446b, according to the EU numbering index.

In one embodiment, the altered antibody variant chain is part of a tetravalent antibody, a minibody, a tetravalent minibody, or a diabody.

In one embodiment, the CH3 domain comprises an engineered cysteine or thiol-containing analog thereof at one or more amino acid positions selected from the group consisting of amino acid positions 355, 359-361, 389, 413, 415, 418, 422, 441, 443, and 446b, according to the EU numbering index.

In another embodiment, the CH3 domain comprises an engineered cysteine or thiol-containing analog thereof at one or more amino acid positions selected from the group consisting of 350, 355, 361, 389, 415, 441, 443, and 446b, according to the EU numbering index.

In another embodiment, the CH3 domain comprises an engineered cysteine or thiol-containing analog thereof at one or more amino acid positions selected from the group consisting of 355, 361, 389, 415, and 446b, according to the EU numbering index.

In another embodiment, the CH3 domain comprises an engineered cysteine or thiol-containing analog thereof at amino acid position 355 or 415, according to the EU numbering index. In a preferred embodiment, the altered polypeptide comprising the CH3 domain is capable of expression by a host cell at a yield of at least 5 mg per liter of host cell culture medium. In a still more preferred embodiment, the altered polypeptide is capable of expression by a host cell at a yield of at least 5 mg per liter of host cell culture medium. In a preferred embodiment, the host cell is a mammalian host cell (e.g. a CHO cell).

In still another embodiment, the altered binding polypeptide comprises at least two engineered cysteine residues or thiol-containing analogs thereof.

In yet another embodiment, the altered polypeptides of the invention are capable of expression by a host cell at a yield of at least 2 mg per liter of host cell culture medium (e.g. at least 2, 3, 4, 5, 6, 7, 8, 9, 10, 11, 12, 13, 14, 15, 20, 25, 50, 75, or 100 mg per liter of host cell culture medium).

In another embodiment, the altered binding polypeptide comprises at least one CH1 domain or portion thereof wherein the CH1 domain comprises an engineered cysteine or thiol-containing analog thereof at one or more amino acid positions selected from the group consisting of amino acid positions 118-215, according to the EU numbering index.

In one embodiment, the CH1 domain comprises an engineered cysteine residue or thiol-containing analog thereof at one or more amino acid positions selected from the group consisting of amino acid positions 132, 138, 164, and 191, according to the EU numbering index.

In still another embodiment the altered binding polypeptide comprises at least one CL domain or portion thereof wherein the CL domain comprises an engineered cysteine or thiol-containing analog thereof at one or more of amino acid positions 108-211, according to the Kabat numbering index.

In one embodiment, the CL domain comprises an engineered cysteine residue or thiol-containing analog thereof at one or more amino acid positions selected from the group consisting of amino acids 122, 127, 158, and 184, according to the Kabat numbering index.

In one embodiment, the altered binding polypeptide comprises a second CH3 domain comprising an engineered cysteine or thiol-containing analog thereof.

In another embodiment, the first and second CH3 domains comprise cysteine or thiol-containing analogs thereof at different amino acid positions.

In yet another embodiment, the first and second CH3 domains comprise cysteine or thiol-containing analogs thereof at the same amino acid positions.

In one embodiment, the CH3 domain is derived from an IgG1 antibody. In another embodiment, the CH3 domain is derived from an IgG4 antibody.

In still another embodiment, the altered binding polypeptide lacks a CH2 domain. In another embodiment, the altered binding polypeptide comprises a CH2 domain. In one embodiment, the CH2 domain comprises at least one engineered cysteine residue or thiol-containing analog thereof. In a more specific embodiment, the CH2 domain comprises an engineered cysteine residue or thiol-containing analog thereof at amino acid position 274 or 324, according to the EU numbering index.

In one embodiment, altered binding polypeptide comprises a complete Fc region.

In one aspect, the invention pertains to an altered binding polypeptide comprising a CH3 domain having an amino acid sequence selected from the group consisting of the amino acid sequences set forth in SEQ ID NO:7-9, wherein the CH3 domain comprises the substitution of an original amino acid at one or more of the amino acids positions selected from the group consisting of amino acid positions 350, 352, 353, 355, 358-366, 368, 370, 371, 389-391, 394-396, 398, 400-407, 409-423 441, 443, 445, 446, and 446b, according to the EU numbering index.

In one embodiment, the original amino acid is solvent-exposed. In another embodiment, the original amino acid has a cysteine-compatible backbone.

In still another aspect, the invention pertains to an altered binding polypeptide comprising at least one of: (i) a CH1 domain or portion thereof comprising an engineered cysteine or thiol-containing analog thereof at one or more amino acid positions selected from the group consisting of amino acid positions 118-215, according to the EU numbering index, and (ii) a CL domain or portion thereof comprising an engineered cysteine or thiol-containing analog thereof at one or more amino acid positions selected from the group consisting of amino acid positions 108-211, according to the Kabat numbering index.

In one embodiment, the CH1 domain comprises an engineered cysteine residue or thiol-containing analog thereof at one or more amino acid positions selected from the group consisting of amino acid positions 132, 138, 164, and 191, according to the EU index.

In another embodiment, the CL domain comprises an engineered cysteine residue or thiol-containing analog thereof at one or more amino acid positions selected from the group consisting of 122, 127, 158, and 184, according to the Kabat numbering index.

In yet another embodiment, the altered binding polypeptide comprises an antigen binding portion of an antibody molecule.

In still another embodiment, the antigen binding portion is a Fab fragment.

In another embodiment, the CH1 domain or the CL domain is fused to a scFv fragment.

In one embodiment, the binding polypeptide comprises at least one binding site selected from the group consisting of an antigen binding site, a ligand binding portion of a receptor, and a receptor binding portion of a ligand.

In one embodiment, the binding polypeptide comprises a first and second binding sites.

In yet another embodiment, the first and second binding sites have the same specificity.

In yet another embodiment, the first and second binding sites have different specificities.

In one embodiment, at least one binding site comprises at least one CDR from antibody selected from the group consisting of CBE11, 14A2, B3F6, 2B8, Lym 1, Lym 2, LL2, Her2, B1, MB1, BH3, B4, B72.3, CC49, and 5E10.

In another embodiment, the invention pertains to a nucleic acid molecule encoding an altered binding polypeptide.

In another embodiment, the invention pertains to a vector comprising a nucleic acid molecule encoding an altered binding polypeptide of the invention. In another embodiment, the invention pertains to a host cell comprising such a vector.

In another embodiment, the invention pertains to an altered binding protein comprising a first and second binding polypeptide wherein the first and second polypeptides are independently selected from the altered binding polypeptides of claims 1, or 3-33.

In one aspect, the invention pertains to an altered polypeptide comprising at least one CH3 domain or portion thereof wherein the CH3 domain comprises an engineered cysteine or thiol-containing analog thereof at one or more amino acid positions selected from the group consisting of amino acid positions 350, 352, 353, 355, 358-366, 368, 370, 371, 389-391, 394-396, 398,400-407, 409-423, 441, 443, 445, 446, or 446b, according to the EU index.

In still another aspect, the invention pertains to an altered polypeptide comprising at least one CH1 domain comprising an engineered cysteine residue or thiol-containing analog thereof at one or more amino acid positions selected from the group consisting of acid positions 132, 138, 164, and 191, according to the EU numbering index.

In another aspect, the invention pertains to an altered polypeptide comprising at least one CH2 domain comprising an engineered cysteine residue or thiol-containing analog thereof amino acid position 274 or 324, according to the EU numbering index.

In yet another aspect, the invention pertains to an altered polypeptide comprising at least one CL domain, wherein the CL domain comprises an engineered cysteine residue or thiol-containing analog thereof at one or more amino acid positions selected from the group consisting of amino acid positions 122, 127, 158, and 184, according to the Kabat numbering index.

In still another aspect, the invention pertains to a method for making a modified binding protein, the method comprising the steps of:
a) contacting an altered binding protein of any one of claims 1-33 or 37 with a thiol-modification reagent; and
b) conjugating the binding protein of step a) with an effector moiety.

In yet another aspect, the invention pertains to a modified binding protein of formula I:

Pro-(S—[Y-E_{q}]ₘ)ₙ (I)

wherein
a. Pro is an altered binding protein of any of the preceding claims;
b. S is a sulfur atom of an engineered cysteine residue or analog thereof of the altered binding protein;
c. Y is a linking moiety or a covalent bond, independently selected for each occurrence;
d. E is an independently selected effector moiety for each occurrence; and
e. q, m and n are each independently selected positive integers for each occurrence.

In one embodiment, the linker moiety is an optionally substituted chain of 1 to 100 carbon, oxygen, nitrogen, and sulfur atoms.

In another embodiment, the bridging moiety comprises a spacer moiety and a linker moiety.

In one embodiment, the linker moiety is non-cleavable. In another embodiment, the linker moiety is cleavable.

In one embodiment, the linker moiety is selected such that it is cleaved extracellularly. In still another embodiment, the linker moiety is selected such it is cleaved intracellularly.

In one embodiment, the linker moiety is selected such that it is cleaved by a drop of pH, enzymatic cleavage or a change in redox potential.

In another embodiment, the linker moiety comprises a disulfide, acetal, ketal, orthoester, trityl, cis-aconityl, thiocarbamoyl, or a peptide moiety.

In still another embodiment, the effector moiety is a therapeutic or diagnostic agent.

In another embodiment, the effector moiety is a therapeutic agent selected from the group consisting of an anti-cancer agent, antibiotic, and an anti-inflammatory agent.

In one embodiment, the therapeutic agent is an anti-cancer agent selected from the group consisting of a cytostatic agent, an enzyme inhibitor, a gene regulator, a cytotoxic nucleoside, a tubulin binding agent, a hormone or hormone antagonist, and an antiangiogenic agent.

In another embodiment, the therapeutic agent is an anti-cancer agent selected from the group consisting of a doxorubicin, a maytansanoid, an etoposide, a taxane, paclitaxel, fluorouracil, mitomycin, camptothecin, a vinca alkaloid, neocarzinostatin, calicheamicin, a maytansinoid, (RS)-cyclophosphamide, 6-mercatopurine, auristatin E, daunorubicin, and a derivative or analog thereof.

In another embodiment, the anti-cancer agent is a maytansinoid having the following formula (II):

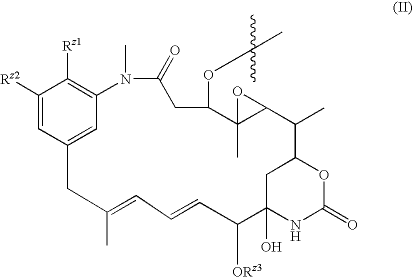

wherein

R^{Z1} is halogen or hydrogen; and

R^{Z2} and R^{Z3} are each hydrogen or lower alkyl.

In one embodiment, the effector moiety further comprises an affinity moiety or a tag moiety.

In another aspect, the invention pertains to a modified binding protein of formula V:

Pro₁-(S—[Y—(S-Pro₂)_{q}]ₘ)ₙ (V)

wherein

Pro₁ and Pro₂ are independently selected from the altered binding proteins of any of claims 1-33;

S is a sulfur atom of an engineered cysteine residue or analog thereof;

Y is a linking moiety or a covalent bond, independently selected for each occurrence;

E is an independently selected effector moiety for each occurrence; and

q, m and n are each independently selected positive integers for each occurrence.

In one embodiment, q is 1, m is 1, n is 2, Pro₁ is an altered CH₃-containing binding protein and Pro₂ is an altered CH1-containing binding protein or an altered CL-containing protein.

In another embodiment, the altered CH3-containing binding protein is an altered antibody.

In yet another embodiment, the altered CH1-containing binding protein or the altered CL-containing protein is an altered Fab fragment.

In one embodiment, the modified binding protein has at least one binding specificity for a TNF ligand family member or a TNF receptor family member.

In one embodiment, q is 1, m is 3, n is 1, Pro₁ is an altered CH1-containing binding protein or CL-containing binding protein, and each Pro₂ is an altered CH1-containing binding protein.

In still another embodiment, the CH1-containing binding protein and the CL-containing binding protein is a Fab fragment.

In yet another embodiment, at least one CH1-containing binding protein has at least one binding specificity for TRAIL-R2.

In another embodiment, at least one CH1-containing binding protein has at least one binding specificity for LTβR.

In yet another aspect, the invention pertains to a method for treating a subject suffering from a disorder that would benefit from treatment with a binding protein, comprising administering to the subject an effective amount of modified binding protein of the invention, such that the subject is treated.

In one embodiment, the subject is suffering from cancer.

In yet another aspect, the invention pertains to a pharmaceutical composition, comprising a modified binding protein of the invention and a pharmaceutically acceptable carrier.

### BRIEF DESCRIPTION OF THE DRAWINGS

FIG. 1 depicts exemplary constant region domain amino acid sequences. FIG. 1A depicts an exemplary CH1 domain amino acid sequence (SEQ ID NO:1). FIGS. 1B-1D depict exemplary CH2 domain amino acid sequences (SEQ ID NOs:2-4). FIGS. 1E-1G depict exemplary CH3 domain amino acid sequences (SEQ ID NOs:5-7).

FIG. 2 depicts structural models of an exemplary Fc region. Panel A shows the location of the FcRn binding loop that extends from aspartate 280 to threonine 299 with relations to FcRn domains. Panel B shows the location of residues in 15 angstrom FcRn contact zone with relations to FcRn domains.

FIG. 3 depicts the amino acid sequences for the variable region of an exemplary altered CBE11 antibody of the invention. FIG. 3A depicts the amino acid sequence of the light chain variable region of the mouse CBE11 antibody (SEQ ID NO:8). FIG. 3B depicts the amino acid sequence of the heavy chain variable region of the mouse CBE11 antibody (SEQ ID NO:9). FIG. 3C depicts the light chain variable region sequence of a humanized CBE11 antibody (SEQ ID NO: 10). FIG. 3D depicts the heavy chain variable region sequence of a humanized CBE11 antibody (SEQ ID NO:11).

FIG. 4 depicts the amino acid sequences for the variable region of an exemplary altered 14A2 antibody of the invention. FIG. 4A depicts the light chain variable region sequence of mouse 14A2 (SEQ ID NO:12). FIG. 4B depicts the heavy chain variable region of mouse 14A2 (SEQ ID NO:13).

FIG. 5 depicts a schematic diagram of an exemplary modified tetravalent antibody of the invention wherein the engineered cysteine residues in the altered CH3 domains of the antibody heavy chain polypeptides are each conjugated to engineered cysteine residues in the CL domains of the Fab fragments via a linking moiety. Other configurations are also possible, for example, the altered CH3 domains of the heavy chain polypeptides may be conjugated to engineered cysteine residues in the CH1 domains of the Fab fragments via the linking moiety. In another embodiment, an altered CH2 domain of the heavy chain polypeptide is conjugated to engineered cysteine residue in the CH1 domain of the Fab fragment via a linking moiety. In another embodiment, one heavy chain polypeptide is conjugated to an altered CH1 domain of a first Fab fragment and the other heavy chain is conjugated to an altered CL domain of a second Fab fragment. In another embodiment, only one of the heavy chain polypeptides is conjugated. The modified tetravalent antibody can also be constructed to be bispecific, where in one Fv region and one Fab have a first specificity (e.g. anti-TRAIL-R2) and the remaining Fv region and Fab have a second specificity (e.g. anti-LTβR). In another embodiment, both Fv regions have a first specificity and both Fabs have a second specificity.

FIG. 6 depicts a schematic diagram of an exemplary modified tetravalent Fab₄ fragment of the invention wherein the engineered cysteine residues in the altered CH1 constant region domains of four altered Fab fragments are conjugated to each other via an exemplary linking moiety of the invention. Other configurations are also possible, for example, one or two of the Fab fragments may comprise an altered CL constant region domain having at least one engineered cysteine residue which is conjugated to the remaining Fab fragments via the linking moiety. The modified tetravalent Fab₄ fragments can also be multispecific. In one embodiment, two Fab fragments have a first binding specificity (e.g. anti-TRAIL-R2) and the remaining Fab fragments have a second binding specificity (e.g. anti-LTβR). In another embodiment, one Fab fragment has a first binding specificity and the remaining Fab fragments have a second binding specificity.

FIG. 7A depicts the amino acid sequence (SEQ ID NO: 14) of an IgG1 Fc region. The location of exemplary amino acid positions within the sequence which may be substituted with a engineered cysteine residue or analog thereof are indicated in bold typeface. FIG. 7B depicts a structural model of the IgG1 Fc region, illustrating the surface positions and distribution of the amino acid positions highlighted in FIG. 7A.

FIG. 8 is an alignment of the amino acid sequences of a CH3 domain (SEQ ID NO: 16), a CL domain (SEQ ID NO:17), and a CH1 domain (SEQ ID NO:15). The location of exemplary amino acid positions which may be substituted with an engineered cysteine residue or analog thereof are indicated in bold typeface.

FIG. 9 depicts the plasmid map of Pichia pastoris expression vector pKS528 employed as site-directed mutagenesis template for the introduction of engineered cysteine residues in a His6-Fc human IgG1 fragment (the 6×His tag is disclosed as SEQ ID NO: 59).

FIG. 10 depicts the amino acid (SEQ ID NO:18) and nucleotide (SEQ ID NO: 19) sequences of the alpha factor-Fc fusion protein mutagenesis template in pKS538. The underlined KR sequence adjacent to the XhoI site, is a site for KEX2 processing, which removes the alpha factor seretion signal. The expected amino-terminus of the secreted Fc protein is GCHHHHHHVVDK (SEQ ID NO: 54). Underlined residues are mutagenesis sites at which engineered cysteine residues were introduced: R355, N361, N389 and S415.

FIG. 11 is a photograph of an SDS-PAGE gel of culture supernatants from recombinant Pichia pastoris strains following induction of protein expression with methanol (the 6×His tag is disclosed as SEQ ID NO: 59). The major protein band in each band is a wild type or altered Fc fragment having an engineered cysteine residue (R355C, N361C, N389C, K447C, S415C).

FIG. 12 is a photograph of an SDS-PAGE gel of culture supernatants from recombinant Pichia pastoris strains following induction of protein expression with doxycycline. The major protein band in each band is a wild type or altered Fc fragment having an engineered cysteine residue (R355C, N361C, N389C, S415C).

FIG. 13 depicts plasmids which were used in a subcloning strategy for the generation of an altered humanized CBE11 antibody. FIG. 13A depicts the EAG1325 plasmid containing the coding region of the CBE11 Heavy Chain. FIG. 13B depicts the pKS532 plasmid containing altered CH3 domain with a R355C mutation. FIG. 13C depicts the PV90 expression plasmid for expression of the altered CBE11 heavy chain in CHO cells.

FIG. 14 depicts the amino acid (SEQ ID NO:20) and nucleotide (SEQ ID NO:21) for the coding region of the CBE11 Heavy Chain in EAG1325. After signal sequence cleavage, the N-terminal sequence is EVQL (SEQ ID NO: 55). The N-terminal E residue is underlined. The SacII and NotI sites used for subcloning are shown.

FIG. 15 depicts a plasmid map of pCCM266. pCCM266 encodes the Heavy Chain of B3F6.

FIG. 16 depicts the amino acid (SEQ ID NO:22) and nucleotide (SEQ ID NO:23) sequences for the coding region of the heavy chain of B3F6. After signal sequence cleavage, the N-terminal sequence is QVQL (SEQ ID NO: 56). The N-terminal Q residue is underlined. The SacII and BsrG1 sites used for subcloning are shown.

FIG. 17 depicts a plasmid map of XW335. pCCM266 encodes the Heavy and Light Chains of humanized CBE11 Fab.

FIG. 18 depicts the amino acid (SEQ ID NO:24) and nucleotide (SEQ ID NO:25) sequences for the coding region of the heavy chain of CBE11 Fab. After signal sequence cleavage, the N-terminal sequence is QVQL (SEQ ID NO: 56). Following signal sequence cleavage, the N-terminus is AVQL- (SEQ ID NO: 57). Residues at which Cys mutations were engineered in the CH1 region are underlined.

FIG. 19 depicts the amino acid (SEQ ID NO:26) and nucleotide (SEQ ID NO:27) sequences for the coding region of the light chain of CBE11 Fab. Following signal sequence cleavage, the N-terminus is DIQM- (SEQ ID NO: 58). Residues at which Cys mutations were engineered in the CL region are underlined.

FIG. 20 is a photograph of a denaturing, non-reducing SDS-PAGE gel of a, modified B3F6 antibody containing an engineered S415C cysteine residue following various chemical treatments. Lanes 1 and 2 show the protein samples which have been treated with N-ethylmaleimide (NEM) or 5K molecular weight PEG-maleimide (5K-PEGM), respectively. Lanes 3 and 4 show protein samples that have been treated with MEA reductant prior to treatment with NEM (lane 3) or 5K-PEGM (lane 4), respectively.

FIGS. 21A and 21B depict different views of a structural model of the IgG1 Fc region, illustrating the surface positions and distribution of exemplary amino acid positions which are substituted according to the invention.

FIG. 22 depicts an alignment of the amino acid sequence of an IgG1 Fc region (SEQ ID NO: 14) and an IgG4 Fe region (SEQ ID NO: 63). Numbering is according to the EU index. The location of exemplary amino acid positions within the IgG1 sequence that may be substituted with an engineered cysteine residue or analog thereof, as well as complementary positions within the IgG4 sequence that may likewise be substituted, are indicated in bold typeface and shading.

### DETAILED DESCRIPTION OF THE INVENTION

The present invention advances the art by providing inter alia altered binding polypeptides and binding proteins comprising such altered binding polypeptides (e.g, antibodies and fusion proteins), which comprise constant regions or portions thereof that are capable of being modified by site-specific conjugation. In particular, the altered polypeptides of the invention feature engineered free cysteines or analogs thereof that facilitate the conjugation of effector moieties that impart novel functionality to the binding polypeptide or protein.

The altered polypeptides of the invention are rationally designed. Unlike previous efforts for site-specific conjugation, the altered polypeptides of the invention do not contain engineered cysteine residues that form interchain or intrachain disulfide bonds which alter the desired properties of the binding protein or facilitate its oligomerization with other binding proteins (e.g. Glockshuber et al., Biochemistry, 29(6): 1362-7, 1990; Shopes, J. Immunol., 148(9): 2918-22, 1992; Caron et al., J. Exp. Med., 176:1191-5, 1992; Shopes, Mol. Immunol., 30(6): 603-9; 1993, Adams et al., Cancer Res., 53(17): 4026-34; Reiter et al., Protein. Eng., 7(5): 697-704, 1994; Kipriyanov et al., Cell Biophys., 26(3): 187-204, 1995; Webber et al., Mol. Immunol., 32(4): 249-58, 1995; WO 91/19515). Furthermore, the altered polypeptides of the invention retain the desirable biological properties conferred by the constant region, e.g. antigen-dependent effector functions such as complement fixation or cytolytic activity.

The invention further provides modified binding polypeptides and methods for using such modified binding polypeptides in therapy and diagnosis. The modified polypeptides comprise one or more constant region domains that have been conjugated with effector moieties at an engineered cysteine residue or an analog or derivative thereof.

### I. DEFINITIONS

As used herein, the term “polypeptide” refers to a polymer of two or more of the natural amino acids or non-natural amino acids. The polypeptides of the invention comprise at least one amino acid sequence derived from an immunoglobulin (Ig) molecule. In one embodiment a polypeptide of the invention comprises an amino acid sequence or one or more moieties not derived from an immunoglobulin molecule. Exemplary modifications are described in more detail below. For example, in one embodiment, a polypeptide of the invention may comprise a flexible linker sequence. In another embodiment, a polypeptide may be modified to add a functional moiety (e.g., PEG, a drug, or a label).

As used herein, the term “protein” refers to a polypeptide or composition comprising more than one polypeptide. Accordingly, proteins may be either monomers or multimers. For example, in one embodiment, a binding protein of the invention is a dimer. In one embodiment, the dimers of the invention are homodimers, comprising two identical monomeric subunits or polypeptides. In another embodiment, the dimers of the invention are heterodimers, comprising two non-identical monomeric subunits or polypeptides. The subunits of the dimer may comprise one or more polypeptide chains. For example, in one embodiment, the dimers comprise at least two polypeptide chains. In one embodiment, the dimers comprise two polypeptide chains. In another embodiment, the dimers comprise four polypeptide chains (e.g., as in the case of antibody molecules).

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence.

Preferred polypeptides of the invention comprise an amino acid sequence derived from a human immunoglobulin sequence. However, polypeptides may comprise one or more amino acids from another mammalian species. For example, a primate heavy chain portion, hinge portion, or binding site may be included in the subject polypeptides. Alternatively, one or more murine amino acids may be present in a polypeptide. Preferred polypeptides of the invention are not immunogenic.

It will also be understood by one of ordinary skill in the art that the polypeptides of the invention may be altered such that they vary in amino acid sequence from the naturally occurring or native polypeptide from which they were derived, while retaining the desirable activity of native polypeptide. For example, nucleotide or amino acid substitutions leading to conservative substitutions or changes at “non-essential” amino acid residues may be made. An isolated nucleic acid molecule encoding a non-natural variant of a polypeptide derived from an immunoglobulin (e.g., an immunoglobulin heavy chain portion or light chain portion) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis.

The polypeptides of the invention may comprise conservative amino acid substitutions at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in polypeptide is preferably replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of the immunoglobulin coding sequence, such as by saturation mutagenesis, and the resultant mutants can be incorporated into polypeptides of the invention and screened for their ability to bind to the desired target.

As used herein, the term “altered polypeptide” includes polypeptides (e.g. binding polypeptides) comprising at least one engineered cysteine residue or an analog or derivative thereof.

A used herein, the term “native cysteine” shall refer to a cysteine amino acid that occurs naturally at a particular amino acid position of a polypeptide and which has not been modified, introduced, or altered by the hand of man. The term “engineered cysteine residue or analog thereof” or “engineered cysteine or analog thereof” shall refer to a non-native cysteine residue or a cysteine analog (e.g. thiol-containing analogs such as thiazoline-4-carboxylic acid and thiazolidine-4 carboxylic acid (thioproline, Th)), which is introduced by synthetic means (e.g. by recombinant techniques, in vitro peptide synthesis, by enzymatic or chemical coupling of peptides or some combination of these techniques) into an amino acid position of a polypeptide that does not naturally contain a cysteine residue or analog thereof at that position.

The term “modified polypeptide”, as used herein, include polypeptides to a composition that has been conjugated or linked wherein the composition is not found conjugated or linked to the polypeptide in nature. In preferred embodiments, the modified polypeptides of the invention are conjugated or linked to a composition at an engineered cysteine or an analog or derivative thereof.

As used herein, the term “binding polypeptide” refers to polypeptides that comprise at least one binding site or binding domain that specifically binds to a target molecule (such as an antigen or binding partner). For example, in one embodiment, a binding polypeptide of the invention comprises an immunoglobulin antigen binding site or the portion of a receptor molecule responsible for ligand binding or the portion of a ligand molecule that is responsible for receptor binding. The binding polypeptides of the invention preferably also comprise at least one amino acid sequence derived from an immunoglobulin (Ig) molecule.

The terms “binding domain” or “binding site”, as used herein, shall refer to a binding polypeptide that mediates specific binding with a target molecule (e.g. an antigen, ligand, receptor, substrate or inhibitor). Exemplary binding domains include an antibody variable domain, a receptor binding domain of a ligand, a ligand binding domain of a receptor or an enzymatic domain. The term “ligand binding domain” as used herein refers to any native receptor (e.g., cell surface receptor) or any region or derivative thereof retaining at least a qualitative ligand binding ability, and preferably the biological activity of a corresponding native receptor. The term “receptor binding domain” as used herein refers to any native ligand or any region or derivative thereof retaining at least a qualitative receptor binding ability, and preferably the biological activity of a corresponding native ligand. In one embodiment, the binding polypeptides of the invention have at least one binding domain specific for a molecule targeted for reduction or elimination, e.g., a cell surface antigen or a soluble antigen. In preferred embodiments, the binding domain is an antigen binding site.

The term “specificity” includes the number of potential binding sites which specifically bind (e.g., immunoreact with) a given target. A binding polypeptide may be monospecific and contain one or more binding sites which specifically bind a target or the binding polypeptide may be multispecific and contain two or more binding sites which specifically bind the same or different targets. In one embodiment, multispecific binding polypeptide having binding specificity for more than one target molecule (e.g., more than one antigen or more than one epitope on the same antigen) can be made. In another embodiment, the multispecific binding polypeptide have at least one binding domain specific for a molecule targeted for reduction or elimination and at least one binding domain specific for a target molecule on a cell. In another embodiment, the multispecific binding polypeptide has at least one binding domain specific for a molecule targeted for reduction or elimination and at least one binding domain specific for a drug. In yet another embodiment, the multispecific binding polypeptide has at least one binding domain specific for a molecule targeted for reduction or elimination and at least one binding domain specific for a prodrug. In a preferred embodiment, the multispecific binding polypeptides are tetravalent antibodies that have two binding domains specific for one target molecule and two binding sites specific for the second target molecule.

The binding polypeptides of the invention comprise at least one binding site. In one embodiment, the binding polypeptides of the invention comprise at least two binding sites. In one embodiment, the binding polypeptides comprise three binding sites. In another embodiment, the binding polypeptides comprise four binding sites. As used herein the term “valency” refers to the number of potential binding domains in a binding polypeptide. Each binding domain specifically binds one target molecule. When a binding polypeptide comprises more than one binding domain, each binding domain may specifically bind the same or different molecules (e.g., may bind to different ligands or different antigens, or different epitopes on the same antigen). Polypeptides comprising two constant region portions may be linked to form two associated Ys so there will be four binding sites forming a “tetravalent” molecule (see e.g., WO02/096948A2)). In another embodiment, tetravalent minibodies or domain deleted antibodies can be made.

As used herein, the term “immunoglobulin” includes a polypeptide having a combination of two heavy and two light chains whether or not it possesses any relevant specific immunoreactivity. As used herein, the term “antibody” refers to such assemblies (e.g., intact antibody molecules, antibody fragments, or variants thereof) which have significant known specific immunoreactive activity to an antigen of interest (e.g. a tumor associated antigen). Antibodies and immunoglobulins comprise light and heavy chains, with or without an interchain covalent linkage between them. Basic immunoglobulin structures in vertebrate systems are relatively well understood.

As will be discussed in more detail below, the generic term “antibody” comprises five distinct classes of antibody that can be distinguished biochemically. All five classes of antibodies are clearly within the scope of the present invention, the following discussion will generally be directed to the IgG class of immunoglobulin molecules. With regard to IgG, immunoglobulins comprise two identical light polypeptide chains of molecular weight approximately 23,000 Daltons, and two identical heavy chains of molecular weight 53,000-70,000. The four chains are joined by disulfide bonds in a “Y” configuration wherein the light chains bracket the heavy chains starting at the mouth of the “Y” and continuing through the variable region.

Light chains of immunoglobulin are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma, mu, alpha, delta, or epsilon, (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, etc. are well characterized and are known to confer functional specialization. Modified versions of each of these classes and isotypes are readily discernable to the skilled artisan in view of the instant disclosure and, accordingly, are within the scope of the instant invention.

Both the light and heavy chains are divided into regions of structural and functional homology. The term “region” refers to a part or portion of an immunoglobulin or antibody chain and includes constant region or variable regions, as well as more discrete parts or portions of said regions. For example, light chain variable regions include “complementarity determining regions” or “CDRs” interspersed among “framework regions” or “FRs”, as defined herein.

The regions of an immunoglobulin heavy or light chain may be defined as “constant” (C) region or “variable” (V) regions, based on the relative lack of sequence variation within the regions of various class members in the case of a “constant region”, or the significant variation within the regions of various class members in the case of a “variable regions”. The terms “constant region” and “variable region” may also be used functionally. In this regard, it will be appreciated that the variable regions of an immunoglobulin or antibody determine antigen recognition and specificity. Conversely, the constant regions of an immunoglobulin or antibody confer important effector functions such as secretion, transplacental mobility, Fc receptor binding, complement binding, and the like. The subunit structures and three dimensional configuration of the constant regions of the various immunoglobulin classes are well known.

The constant and variable regions of immunoglobulin heavy and light chains are folded into domains. The term “domain” refers to a globular region of a heavy or light chain polypeptide comprising peptide loops (e.g., comprising 3 to 4 peptide loops) stabilized, for example, by β-pleated sheet and/or intrachain disulfide bond. Constant region domains on the light chain of an immunoglobulin are referred to interchangeably as “light chain constant region domains”, “CL regions” or “CL domains”. Constant domains on the heavy chain (e.g. hinge, CH₁, CH₂ or CH₃ domains) are referred to interchangeably as “heavy chain constant region domains”, “CH” region domains or “CH domains”. Variable domains on the light chain are referred to interchangeably as “light chain variable region domains”, “VL region domains or “VL domains”. Variable domains on the heavy chain are referred to interchangeably as “heavy chain variable region domains”, “VH region domains” or “VH domains”.

By convention the numbering of the variable constant region domains increases as they become more distal from the antigen binding site or amino-terminus of the immunoglobulin or antibody. The N-terminus of each heavy and light immunoglobulin chain is a variable region and at the C-terminus is a constant region; the CH₃ and CL domains actually comprise the carboxy-terminus of the heavy and light chain, respectively. Accordingly, the domains of a light chain immunoglobulin are arranged in a VL-CL orientation, while the domains of the heavy chain are arranged in the VH—CH1-hinge-CH2-CH3 orientation.

Amino acid positions in a heavy chain constant region, including amino acid positions in the CH1, hinge, CH2, and CH3 domains, are numbered herein according to the EU index numbering system (see Kabat et al., in “Sequences of Proteins of Immunological Interest”, U.S. Dept. Health and Human Services, 5^{th} edition, 1991). In contrast, amino acid positions in a light chain constant region (e.g. CL domains) are numbered herein according to the Kabat index numbering system (see Kabat et al., ibid).

As used herein, the term “V_{H} domain” includes the amino terminal variable domain of an immunoglobulin heavy chain, and the term “V_{L} domain” includes the amino terminal variable domain of an immunoglobulin light chain.

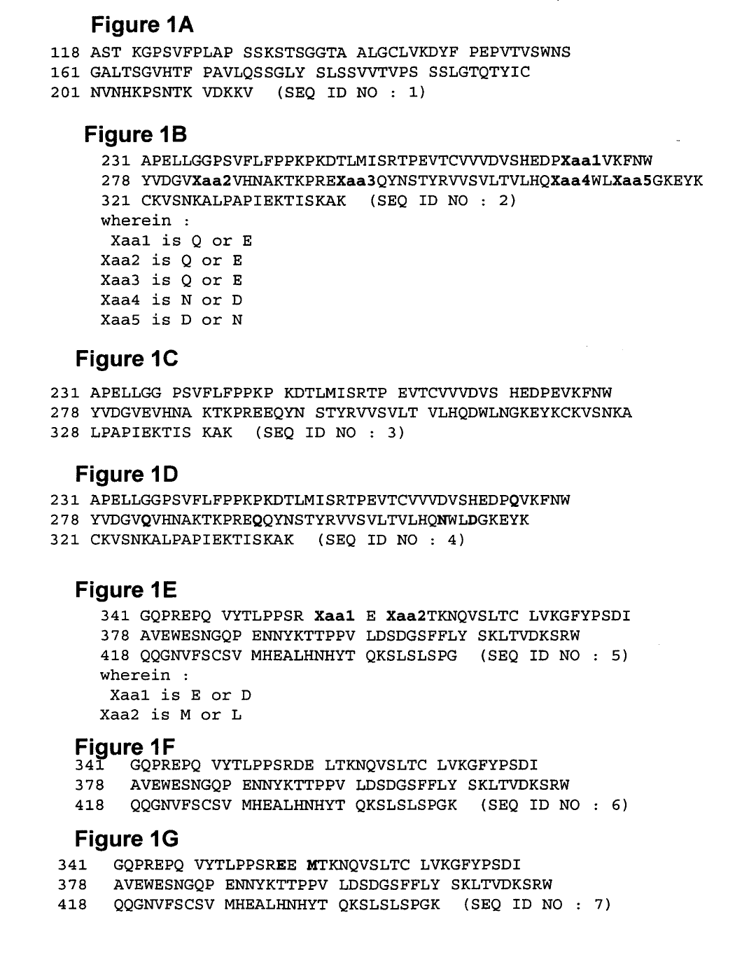

As used herein, the term “CH1 domain” includes the first (most amino terminal) constant region domain of an immunoglobulin heavy chain that extends, e.g., from about EU positions 118-215. The CH1 domain is adjacent to the V_{H} domain and amino terminal to the hinge region of an immunoglobulin heavy chain molecule, and does not form a part of the Fc region of an immunoglobulin heavy chain. In one embodiment, an altered polypeptide of the invention comprises a CH1 domain derived from an immunoglobulin heavy chain molecule (e.g., a human IgG1 or IgG4 molecule). In one embodiment, an altered polypeptide of the invention comprises a CH1 domain (EU positions 118-215) having the amino acid sequence depicted in FIG. 1A (SEQ ID NO:1) or a portion thereof.

As used herein, the term “hinge region” includes the portion of a heavy chain molecule that joins the CH1 domain to the CH2 domain. This hinge region comprises approximately 25 residues and is flexible, thus allowing the two N-terminal antigen binding regions to move independently. Hinge regions can be subdivided into three distinct domains: upper, middle, and lower hinge domains (Roux et al. J. Immunol. 1998, 161:4083).

As used herein, the term “CH2 domain” includes the portion of a heavy chain immunoglobulin molecule that extends, e.g., from about EU positions 231-340. The CH2 domain is unique in that it is not closely paired with another domain. Rather, two N-linked branched carbohydrate chains are interposed between the two CH2 domains of an intact native IgG molecule. In one embodiment, an altered polypeptide of the invention comprises a CH2 domain derived from an IgG1 molecule (e.g. a human IgG1 molecule). In another embodiment, an altered polypeptide of the invention comprises a CH2 domain derived from an IgG4 molecule (e.g., a human IgG4 molecule). In an exemplary embodiment, an polypeptide of the invention comprises a CH2 domain (EU positions 231-340) having the amino acid sequence depicted in FIG. 1B (SEQ ID NO:2), or a portion thereof. In another embodiment, an altered polypeptide of the invention comprises the CH2 domain depicted in FIG. 1C (SEQ ID NO:3) or a portion thereof. In another embodiment, an altered polypeptide of the invention comprises the CH2 domain depicted in FIG. 1D (SEQ ID NO:4) or a portion thereof.

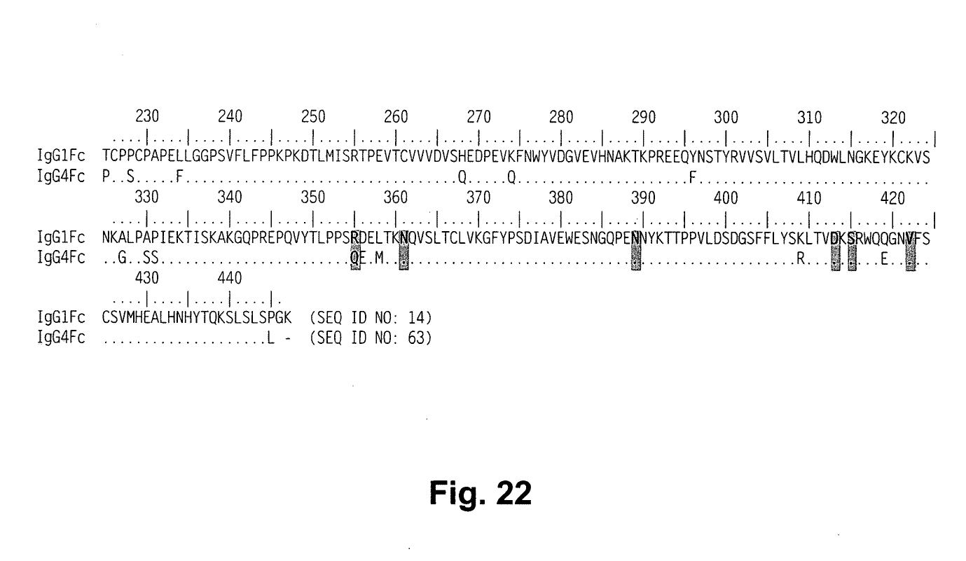

As used herein, the term “CH3 domain” includes the portion of a heavy chain immunoglobulin molecule that extends approximately 110 residues from N-terminus of the CH2 domain, e.g., from about position 341-446b (EU numbering system). The CH3 domain typically forms the C-terminal portion of the antibody. In some immunoglobulins, however, additional domains may extend from CH3 domain to form the C-terminal portion of the molecule (e.g. the CH4 domain in the μ chain of IgM and the ε chain of IgE). In one embodiment, an altered polypeptide of the invention comprises a CH3 domain derived from an IgG1 molecule (e.g., a human IgG1 molecule). In another embodiment, an altered polypeptide of the invention comprises a CH3 domain derived from an IgG4 molecule (e.g., a human IgG4 molecule). In another embodiment, an altered polypeptide of the invention comprises a CH3 domain (EU positions 341-446b) having the amino acid sequence depicted in FIG. 1E (SEQ ID NO:5) or a portion thereof. In one embodiment, the altered polypeptide comprises the CH3 domain depicted in FIG. 1F (SEQ ID NO:6) or a portion thereof. In another embodiment, the altered polypeptide comprises the CH3 domain depicted in FIG. 1G (SEQ ID NO:7) or a portion thereof. In another embodiment, the altered polypeptide comprises the CH3 domain of the IgG4 Fc region depicted in FIG. 22 (SEQ ID NO: 63).

As used herein, the term “CL domain” includes the first (most amino terminal) constant region domain of an immunoglobulin light chain that extends, e.g. from about Kabat position 107A-216. The CL domain is adjacent to the V_{L} domain. In one embodiment, an altered polypeptide of the invention comprises a CL domain derived from a kappa light chain (e.g., a human kappa light chain).

As used herein, the term “Fc region” shall be defined as the portion of a heavy chain constant region beginning in the hinge region just upstream of the papain cleavage site (i.e. residue 216 in IgG, taking the first residue of heavy chain constant region to be 114) and ending at the C-terminus of the antibody. Accordingly, a complete Fc region comprises at least a hinge domain, a CH2 domain, and a CH3 domain.

As used herein, the term “effector function” refers to the functional ability of the Fc region or portion thereof to bind proteins and/or cells of the immune system and mediate various biological effects. Effector functions may be antigen-dependent or antigen-independent.

As used herein, the term “antigen-dependent effector function” refers to an effector function which is normally induced following the binding of an antibody to a corresponding antigen. Typical antigen-dependent effector functions include the ability to bind a complement protein (e.g. C1q). For example, binding of the C1 component of complement to the Fc region can activate the classical complement system leading to the opsonisation and lysis of cell pathogens, a process referred to as complement-dependent cytotoxicity (CDCC). The activation of complement also stimulates the inflammatory response and may also be involved in autoimmune hypersensitivity.

Other antigen-dependent effector functions are mediated by the binding of antibodies, via their Fc region, to certain Fc receptors (“FcRs”) on cells. There are a number of Fc receptors which are specific for different classes of antibody, including IgG (gamma receptors, or IgγRs), IgE (epsilon receptors, or IgεRs), IgA (alpha receptors, or IgαRs) and IgM (mu receptors, or IgμRs). Binding of antibody to Fc receptors on cell surfaces triggers a number of important and diverse biological responses including endocytosis of immune complexes, engulfment and destruction of antibody-coated particles or microorganisms (also called antibody-dependent phagocytosis, or ADCP), clearance of immune complexes, lysis of antibody-coated target cells by killer cells (called antibody-dependent cell-mediated cytotoxicity, or ADCC), release of inflammatory mediators, regulation of immune system cell activation, placental transfer and control of immunoglobulin production.

Certain Fc receptors, the Fc gamma receptors (FcγRs), play a critical role in either abrogating or enhancing immune recruitment. FcγRs are expressed on leukocytes and are composed of three distinct classes: FcγRI, FcγRII, and FcγRIII (Gessner et al., Ann. Hematol., (1998), 76: 231-48). Structurally, the FcγRs are all members of the immunoglobulin superfamily, having an IgG-binding α-chain with an extracellular portion composed of either two or three Ig-like domains. Human FcγRI (CD64) is expressed on human monocytes, exhibits high affinity binding (Ka=10⁸-10⁹ M⁻¹) to monomeric IgG1, IgG3, and IgG4. Human FcγRII (CD32) and FcγRIII (CD16) have low affinity for IgG1 and IgG3 (Ka<10⁷ M⁻¹), and can bind only complexed or polymeric forms of these IgG isotypes. Furthermore, the FcγRII and FcγRIII classes comprise both “A” and “B” forms. FcγRIIa (CD32a) and FcγRIIIa (CD16a) are bound to the surface of macrophages, NK cells and some T cells by a transmembrane domain while FcγRIIb (CD32b) and FcγRIIIb (CD16b) are selectively bound to cell surface of granulocytes (e.g. neutrophils) via a phosphatidyl inositol glycan (GPI) anchor. The respective murine homologs of human FcγRI, FcγRII, and FcγRIII are FcγRIIa, FcγRIIb/1, and FcγRlo.

As used herein, the term “antigen-independent effector function” refers to an effector function which may be induced by an antibody, regardless of whether it has bound its corresponding antigen. Typical antigen-independent effector functions include cellular transport, circulating half-life and clearance rates of immunoglobulins, and facilitation of purification. A structurally unique Fc receptor, the “neonatal Fc receptor” or “FcRn”, also known as the salvage receptor, plays a critical role in regulating half-life and cellular transport. Other Fc receptors purified from microbial cells (e.g. Staphylococcal Protein A or G) are capable of binding to the Fc region with high affinity and can be used to facilitate the purification of the Fc-containing polypeptide.

Unlike FcγRs which belong to the Immunoglobulin superfamily, human FcRns structurally resemble polypeptides of Major Histoincompatibility Complex (MHC) Class I (Ghetie and Ward, Immunology Today, (1997), 18(12): 592-8). FcRn is typically expressed as a heterodimer consisting of a transmembrane a or heavy chain in complex with a soluble β or light chain (β2 microglobulin). FcRn shares 22-29% sequence identity with Class I MHC molecules has a non-functional version of the MHC peptide binding groove (Simister and Mostov, Nature, (1989), 337: 184-7. Like MHC, the α chain of FcRn consists of three extracellular domains (α1, α2, α3) and a short cytoplasmic tail anchors the protein to the cell surface. The α1 and α2 domains interact with FcR binding sites in the Fc region of antibodies (Raghavan et al., Immunity, (1994), 1: 303-15).

FcRn is expressed in the maternal placenta or yolk sac of mammals and it is involved in transfer of IgGs from mother to fetus. FcRn is also expressed in the small intestine of rodent neonates, where it is involved in the transfer across the brush border epithelia of maternal IgG from ingested colostrum or milk. FcRn is also expressed in numerous other tissues across numerous species, as well as in various endothelial cell lines. It is also expressed in human adult vascular endothelium, muscle vasculature, and hepatic sinusoids. FcRn is thought to play an additional role in maintaining the circulatory half-life or serum levels of IgG by binding it and recycling it to the serum. The binding of FcRn to IgG molecules is strictly pH-dependent with an optimum binding at a pH of less than 7.0.

The term “domain-deleted antibody” includes immunoglobulins, antibodies, and immunoreactive fragments or recombinants thereof, in which at least a fraction of one or more of the constant region domains has been deleted or otherwise altered so as to provide desired biochemical characteristics such as the ability to non-covalently dimerize, increased ability to localize at the site of a tumor, or reduced serum half-life when compared with a whole, unaltered antibody of approximately the same immunogenicity.

As indicated above, the variable regions of an antibody allow it to selectively recognize and specifically bind epitopes on antigens. That is, the V_{L} domain and V_{H} domain of an antibody combine to form the variable region (Fv) that defines a three dimensional antigen binding site. This quaternary antibody structure forms the antigen binding site present at the end of each arm of the Y. More specifically, the antigen binding site is defined by three complementary determining regions (CDRs) on each of the heavy and light chain variable regions.

As used herein, the term “antigen binding site” includes a site that specifically binds (immunoreacts with) an antigen (e.g., a cell surface or soluble antigen). The antigen binding site includes an immunoglobulin heavy chain and light chain variable region and the binding site formed by these variable regions determines the specificity of the antibody. An antigen binding site is formed by variable regions that vary from one polypeptide to another. The altered antibodies of the invention comprise at least two antigen binding sites.

Altered antibodies of the instant invention comprise at least two antigen binding domains that provide for the association of the antibody with the selected antigen. The antigen binding domains need not be derived from the same immunoglobulin molecule. In this regard, the variable region may or be derived from any type of animal that can be induced to mount a humoral response and generate immunoglobulins against the desired antigen. As such, the variable region of the altered antibody may be, for example, of mammalian origin e.g., may be human, murine, non-human primate (such as cynomolgus monkeys, macaques, etc.), lupine, camelid (e.g., from camels, llamas and related species).

In naturally occurring antibodies, the six CDRs present on each monomeric antibody are short, non-contiguous sequences of amino acids that are specifically positioned to form the antigen binding site as the antibody assumes its three dimensional configuration in an aqueous environment. The remainder of the heavy and light variable domains show less inter-molecular variability in amino acid sequence and are termed the framework regions. The framework regions largely adopt a β-sheet conformation and the CDRs form loops which connect, and in some cases form part of, the β-sheet structure. Thus, these framework regions act to form a scaffold that provides for positioning the six CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to the immunoreactive antigen epitope.

The term “antibody variant” includes an antibody which does not occur in nature and which has an amino acid sequence or amino acid side chain chemistry which differs from that of a naturally-derived antibody by at least one amino acid or amino acid modification as described herein. As used herein, the term “antibody variant” includes synthetic forms of antibodies which are altered such that they are not naturally occurring, e.g., antibodies that comprise at least two heavy chain portions but not two complete heavy chains (such as, domain deleted antibodies or minibodies); multispecific forms of antibodies (e.g., bispecific, trispecific, etc.) altered to bind to two or more different antigens or to different epitopes on a single antigen); heavy chain molecules joined to scFv molecules; single-chain antibodies; diabodies; and antibodies with altered effector function and the like. ScFv molecules are known in the art and are described, e.g., in U.S. Pat. No. 5,892,019.

As used herein the term “disulfide bond” includes the covalent bond formed between two sulfur atoms. The amino acid cysteine comprises a thiol group that can form a disulfide bond or bridge with a second thiol group. In most naturally occurring IgG molecules, the CH1 and CL regions are linked by native disulfide bonds and the two heavy chains are linked by two native disulfide bonds at positions corresponding to 239 and 242 using the Kabat numbering system (position 226 or 229, EU numbering system).

As used herein, the term “bonded cysteine” shall refer to a native or engineered cysteine residue within a polypeptide which forms a disulfide bond or other covalent bond with a second native or engineered cysteine or other residue present within the same or different polypeptide. An “intrachain bonded cysteine” shall refer to a bonded cysteine that is covalently bonded to a second cysteine present within the same polypeptide (ie. an intrachain disulfide bond). An “interchain bonded cysteine” shall refer to a bonded cysteine that is covalently bonded to a second cysteine present within a different polypeptide (ie. an interchain disulfide bond).

As used herein, the term “free cysteine” refers to a native or engineered cysteine amino acid residues within a polypeptide sequence (and analogs or mimetics thereof, e.g. thiazoline-4-carboxylic acid and thiazolidine-4 carboxylic acid (thioproline, Th)) that exists in a substantially reduced form. Free cysteines are preferably capable of being modified with an effector moiety of the invention.

The term “thiol modification reagent” shall refer to a chemical agent that is capable of selectively reacting with the thiol group of an engineered cysteine residue or analog thereof in an altered polypeptide, and thereby providing means for site-specific chemical addition or crosslinking of effector moieties to the altered polypeptide. Preferably the thiol modification reagent exploits the thiol or sulfhydryl functional group which is present in a free cysteine residue. Exemplary thiol modification reagents include maleimides, alkyl and aryl halides, α-haloacyls, and pyridyl disulfides.

The term “linking moiety” includes moieties which are capable of linking the effector moiety (or optionally an affinity or PEGylation moiety) to the remainder of the modified peptide. In certain embodiments where Z is hydrogen or an amino acid side chain, the linking moiety may be a covalent bond. The linking moiety may be selected such that it is cleavable or non-cleavable. Uncleavable linking moieties generally have high systemic stability, but may also have unfavorable pharmacokinetics. It should be noted that for certain Z moieties, such as affinity moieties and tag moieties, non-cleavable linking moieties may be preferred.

The term “spacer moiety” may be an optionally substituted chain of 0 to 100 atoms, selected from carbon, oxygen, nitrogen, sulfur, etc. In one embodiment, the spacer moiety is selected such that it is water soluble. In another embodiment, the spacer moiety is polyalkylene glycol, e.g., polyethylene glycol or polypropylene glycol.

The terms “PEGylation moiety” or “PEG moiety” includes a polyalkylene glycol compound or a derivative thereof, with or without coupling agents or derivitization with coupling or activating moieties (e.g., with thiol, triflate, tresylate, azirdine, oxirane, or preferably with a maleimide moiety, e.g., PEG-maleimide). Other appropriate polyalkylene glycol compounds include, maleimido monomethoxy PEG, activated PEG polypropylene glycol, but also charged or neutral polymers of the following types: dextran, colominic acids, or other carbohydrate based polymers, polymers of amino acids, and biotin derivatives.

As used herein, the term “effector moiety” (E) may comprise diagnostic and therapeutic agents (e.g. proteins, nucleic acids, lipids, drug moieties, and fragments thereof) with biological or other functional activity. For example, a modified binding polypeptide comprising an effector moiety conjugated to an altered polypeptide has at least one additional function or property as compared to the unconjugated polypeptide. For example, the conjugation of a cytotoxic drug moiety (e.g., an effector moeity) to an altered antibody results in the formation of a modified antibody with drug cytotoxicity as second function (i.e. in addition to antigen binding). In another example, the conjugation of a second altered binding polypeptide to the altered antibody may confer additional binding properties.

In one aspect, wherein the effector moiety is a genetically encoded therapeutic or diagnostic protein or nucleic acid, the effector moiety may be synthesized or expressed by either peptide synthesis or recombinant DNA methods that are well known in the art. In another aspect, wherein the effector is a non-genetically encoded peptide, or a drug moiety, the effector moiety may be synthesized artificially or purified from a natural source.

As used herein, the term “drug moiety” includes anti-inflammatory, anticancer, anti-infective (e.g., anti-fungal, antibacterial, anti-parasitic, anti-viral, etc.), and anesthetic therapeutic agents. In a further embodiment, the drug moiety is an anticancer or cytotoxic agent. Compatible drug moieties may also comprise prodrugs.

As used herein, the term “prodrug” refers to a precursor or derivative form of a pharmaceutically active agent that is less active, reactive or prone to side effects as compared to the parent drug and is capable of being enzymatically activated or otherwise converted into a more active form in vivo. Prodrugs compatible with the invention include, but are not limited to, phosphate-containing prodrugs, amino acid-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. One skilled in the art may make chemical modifications to the desired drug moiety or its prodrug in order to make reactions of that compound more convenient for purposes of preparing modified binding proteins of the invention. The drug moieties also include derivatives, pharmaceutically acceptable salts, esters, amides, and ethers of the drug moieties described herein. Derivatives include modifications to drugs identified herein which may improve or not significantly reduce a particular drug's desired therapeutic activity.

As used herein, the term “anticancer agent” include agents which are detrimental to the growth and/or proliferation of neoplastic or tumor cells and may act to reduce, inhibit or destroy malignancy. Examples of such agents include, but are not limited to, cytostatic agents, alkylating agents, antibiotics, cytotoxic nucleosides, tubulin binding agents, hormones and hormone antagonists, and the like. Any agent that acts to retard or slow the growth of immunoreactive cells or malignant cells is within the scope of the present invention.

An “affinity tag” or an “affinity moiety” is a chemical moiety that is attached to one or more of the altered polypeptide or effector moiety in order to facilitate its separation from other components during a purification procedure. Exemplary affinity domains include the His tag, chitin binding domain, maltose binding domain, biotin, and the like.

An “affinity resin” is a chemical surface capable of binding the affinity domain with high affinity to facilitate separation of the protein bound to the affinity domain from the other components of a reaction mixture. Affinity resins can be coated on the surface of a solid support or a portion thereof. Alternatively, the affinity resin can comprise the solid support its. Such solid supports can include a suitably modified chromatography column, microtiter plate, bead, or biochip (e.g. glass wafer). Exemplary affinity resins are comprised of nickel, chitin, amylase, and the like.

The term “vector” or “expression vector” is used herein for the purposes of the specification and claims, to mean vectors used in accordance with the present invention as a vehicle for introducing into and expressing a desired polynucleotide in a cell. As known to those skilled in the art, such vectors may easily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

For the purposes of this invention, numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV) or SV40 virus. Others involve the use of polycistronic systems with internal ribosome binding sites. Exemplary vectors include those described in U.S. Pat. Nos. 6,159,730 and 6,413,777, and U.S. Patent Application No. 2003 0157641 A1. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by cotransformation. In one embodiment, an inducible expression system can be employed. Additional elements may also be needed for optimal synthesis of mRNA. These elements may include signal sequences, splice signals, as well as transcriptional promoters, enhancers, and termination signals. In one embodiment, a secretion signal, e.g., any one of several well characterized bacterial leader peptides (e.g., pelB, phoA, or ompA), can be fused in-frame to the N terminus of a polypeptide of the invention to obtain optimal secretion of the polypeptide. (Lei et al. (1988), Nature, 331:543; Better et al. (1988) Science, 240:1041; Mullinax et al., (1990). PNAS, 87:8095).

The term “host cell” refers to a cell that has been transformed with a vector constructed using recombinant DNA techniques and encoding at least one heterologous gene. In descriptions of processes for isolation of proteins from recombinant hosts, the terms “cell” and “cell culture” are used interchangeably to denote the source of protein unless it is clearly specified otherwise. In other words, recovery of protein from the “cells” may mean either from spun down whole cells, or from the cell culture containing both the medium and the suspended cells. The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3x63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature. The polypeptides of the invention can also be expressed in non-mammalian cells such as bacteria or yeast or plant cells. In this regard it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides typically become part of inclusion bodies. The polypeptides must be isolated, purified and then assembled into functional molecules. Where tetravalent forms of antibodies are desired, the subunits will then self-assemble into tetravalent antibodies (WO02/096948A2).

In addition to prokaryotes, eukaryotic microbes may also be used. Saccharomyces cerevisiae, or common baker's yeast, is the most commonly used among eukaryotic microorganisms although a number of other strains are commonly available including Pichia pastoris. For expression in Saccharomyces, the plasmid YRp7, for example, (Stinchcomb et al., (1979), Nature, 282:39; Kingsman et al., (1979), Gene, 7:141; Tschemper et al., (1980), Gene, 10:157) is commonly used. This plasmid already contains the TRP1 gene which provides a selection marker for a mutant strain of yeast lacking the ability to grow in tryptophan, for example ATCC No. 44076 or PEP4-1 (Jones, (1977), Genetics, 85:12). The presence of the trpl lesion as a characteristic of the yeast host cell genome then provides an effective environment for detecting transformation by growth in the absence of tryptophan.

In vitro production allows scale-up to give large amounts of the desired altered binding polypeptides of the invention. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose or affinity chromatography.

As used herein, “tumor-associated antigens” means any antigen which is generally associated with tumor cells, i.e., occurring at the same or to a greater extent as compared with normal cells. More generally, tumor associated antigens comprise any antigen that provides for the localization of immunoreactive antibodies at a neoplastic cell irrespective of its expression on non-malignant cells. Such antigens may be relatively tumor specific and limited in their expression to the surface of malignant cells. Alternatively, such antigens may be found on both malignant and non-malignant cells. The altered and modified binding polypeptides of the present invention preferably bind to tumor-associated antigens. Accordingly, the altered and modified binding polypeptides of the present invention may be derived, generated or fabricated from any one of a number of antibodies that react with tumor associated molecules.

As used herein, the term “malignancy” refers to a non-benign tumor or a cancer. As used herein, the term “cancer” includes a malignancy characterized by deregulated or uncontrolled cell growth. Exemplary cancers include: carcinomas, sarcomas, leukemias, and lymphomas. The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

As used herein, the phrase “subject that would benefit from administration of a binding protein” includes subjects, such as mammalian subjects, that would benefit from administration of altered binding polypeptides used, e.g., for detection of an antigen recognized by an altered and modified binding polypeptide (e.g., for a diagnostic procedure) and/or from treatment with an altered binding polypeptide to reduce or eliminate the target recognized by the altered binding polypeptide. For example, in one embodiment, the subject may benefit from reduction or elimination of a soluble or particulate molecule from the circulation or serum (e.g., a toxin or pathogen) or from reduction or elimination of a population of cells expressing the target (e.g., tumor cells). As described in more detail herein, the altered binding polypeptide can be used in unconjugated form or can be conjugated, e.g., to a drug, prodrug, or an isotope.

### II. ALTERED POLYPEPTIDES COMPRISING A CONSTANT REGION DOMAIN

In one aspect, the invention provides altered polypeptides comprising at least one constant region or portion thereof derived from an immunoglobulin molecule. Preferred altered polypeptides of the invention comprise at least a constant region domain or portion thereof having at least one engineered cysteine residue or analog thereof. Cysteine residues or analogs thereof may be introduced, for example by insertion or amino acid substitution, using methods that well known in the art. In one embodiment, the engineered cysteine residue is located in a constant region portion derived from the light chain of an immunoglobulin molecule (e.g. a CL domain or portion thereof). In another embodiment, the engineered cysteine residue or analog thereof is located in a constant region domain derived from the heavy chain of an immunoglobulin molecule (e.g. a CH domain or portion thereof).

Constant region domain sequences useful for producing the altered polypeptides of the present invention may be obtained from a number of different sources. In preferred embodiments, the constant region domain or portion thereof of the altered polypeptide is derived from a human immunoglobulin. It is understood, however, that the constant region domain or portion thereof may be derived from an immunoglobulin of another mammalian species, including for example, a rodent (e.g. a mouse, rat, rabbit, guinea pig) or non-human primate (e.g. chimpanzee, macaque) species. Moreover, the altered polypeptide constant region domain or portion thereof may be derived from any immunoglobulin class, including IgM, IgG, IgD, IgA and IgE, and any immunoglobulin isotype, including IgG1, IgG2, IgG3 and IgG4. In a preferred embodiment, the human isotype IgG1 is used.

A variety of constant region gene sequences (e.g. human constant region gene sequences) are available in the form of publicly accessible deposits. Constant region domains can be selected having a particular effector function (or lacking a particular effector function) or with a particular modification to reduce immunogenicity. Many sequences of antibodies and antibody-encoding genes have been published and suitable constant region domain sequences (e.g. CH1, hinge, CH2, CH3, or CL sequences) or portions thereof can be deroved from these sequences using art recognized techniques. The genetic material obtained using any of the foregoing methods may then be altered or synthesized to provide obtain polypeptides of the present invention. It will further be appreciated that the scope of this invention encompasses alleles, variants and mutations of constant region DNA sequences.

Constant region domains can be cloned, e.g., using the polymerase chain reaction and primers which are selected to amplify the domain of interest. To clone a constant region domain from an antibody, mRNA can be isolated from hybridoma, spleen, or lymph cells, reverse transcribed into DNA, and antibody genes amplified by PCR. PCR amplification methods are described in detail in U.S. Pat. Nos. 4,683,195; 4,683,202; 4,800,159; 4,965,188; and in, e.g., “PCR Protocols: A Guide to Methods and Applications” Innis et al. eds., Academic Press, San Diego, Calif. (1990); Ho et al. 1989. Gene 77:51; Horton et al. 1993. Methods Enzymol. 217:270). PCR may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes. Numerous primer sets suitable for amplification of antibody genes are known in the art (e.g., 5′ primers based on the N-terminal sequence of purified antibodies (Benhar and Pastan. 1994. Protein Engineering 7:1509); rapid amplification of cDNA ends (Ruberti, F. et al. 1994. J. Immunol. Methods 173:33); antibody leader sequences (Larrick et al. 1989 Biochem. Biophys. Res. Commun. 160:1250). The cloning of antibody sequences is further described in Newman et al., U.S. Pat. No. 5,658,570, filed Jan. 25, 1995, which is incorporated by reference herein.

The altered polypeptide may comprise any number of constant region domains of different types. In one embodiment, the altered polypeptide of the invention comprises at least one CH1 domain or portion thereof. In another embodiment, the altered polypeptide of the invention comprises at least one CH2 domain or portion thereof. In another embodiment, the altered polypeptide of the invention comprises at least one CH3 domain or portion thereof. In another embodiment, the altered polypeptide of the invention comprises at least one CH1 domain or portion thereof and at least one CH2 domain or portion thereof. In another embodiment, the altered polypeptide of the invention comprises at least one CH2 domain or portion thereof and at least one CH3 domain or portion thereof. In another embodiment, the altered polypeptide of the invention comprises at least one CH1 domain or portion thereof, at least one CH2 domain or portion thereof, and least one CH3 domain or portion thereof, for example in the orientation CH1-CH2-CH3.

In certain embodiments, the altered polypeptide comprises a whole constant region derived from an immunoglobulin light chain. In other embodiments, the altered polypeptide comprises a whole constant region derived from an immunoglobulin heavy chain. In other embodiments, the altered polypeptide comprises a whole Fc region derived from an immunoglobulin heavy chain, including hinge, CH2, and CH3 domains.

In another embodiment, an altered polypeptide of the invention comprises a complete CH3 domain (about amino acids 341-438 of an antibody Fc region according to EU numbering). In another embodiment, an altered polypeptide of the invention comprises a complete CH2 domain (about amino acids 231-340 of an antibody Fc region according to EU numbering). In another embodiment, an altered polypeptide of the invention comprises at least a CH3 domain, and at least one of a hinge region (about amino acids 216-230 of an antibody Fc region according to EU numbering), and a CH2 domain. In one embodiment, an altered polypeptide of the invention comprises a hinge and a CH3 domain. In another embodiment, an altered polypeptide of the invention comprises a hinge, a CH₂, and a CH₃ domain.

The constant region domains or portions thereof making up the constant region of an altered polypeptide may be derived from different immunoglobulin molecules. For example, a polypeptide may comprise a CH2 domain or portion thereof derived from an IgG1 molecule and a CH3 region or portion thereof derived from an IgG3 molecule. In another example, an altered polypeptide can comprise a CH1 domain derived, in part, from an IgG1 molecule and, in part, from an IgG3 molecule. As set forth above, it will be understood by one of ordinary skill in the art that the constant region domains may be altered such that they vary in amino acid sequence from a naturally occurring antibody molecule.

In another embodiment, an altered polypeptide of the invention comprises at least a CH₃ portion of an Fc region together with a portion sufficient to confer FcRn binding. For example, the portion of the Fc region that binds to FcRn comprises from about amino acids 282-438 of IgG1, EU numbering. Fc regions or FcRn binding portions thereof may be derived from heavy chains of any isotype, including IgG1, IgG2, IgG3 and IgG4. In one embodiment, an Fc region domain from an antibody of the human isotype IgG1 is used. In another embodiment, an Fc region domain from an antibody of the human isotype IgG4 is used.

In one embodiment, an altered polypeptide of the invention comprises a altered synthetic constant regions wherein or more constant region domains are partially or entirely deleted (“domain-deleted constant regions”). In a certain preferred embodiments altered polypeptides of the invention will comprise domain deleted constructs or variants wherein the entire CH2 domain has been removed (ΔCH2 constructs). Those skilled in the art will appreciate that such constructs are particularly preferred due to the regulatory properties of the CH2 domain on the catabolic rate of the antibody. Particularly preferred altered polypeptides comprise CH2 domain-deleted constant regions derived from a vector (e.g., from IDEC Pharmaceuticals, San Diego) encoding an IgG₁ human constant region domain (see, e.g., WO 02/060955A2 and WO02/096948A2). This exemplary vector is engineered to delete the CH2 domain and provide a synthetic vector expressing a domain deleted IgG₁ constant region. This vector may be further modified to encode at least one engineered cysteine residue or analog thereof in the remaining constant region sequence. It will be noted that these exemplary constructs are preferably engineered to fuse an altered CH3 domain directly to a hinge region of the respective polypeptides of the invention.

In other constructs it may be desirable to provide a peptide spacer between the hinge region and the synthetic CH2 and/or CH3 domains. For example, compatible constructs could be expressed wherein the CH2 domain has been deleted and the remaining CH3 domain (synthetic or unsynthetic) is joined to the hinge region with a 5-20 amino acid peptide spacer. Such a peptide spacer may be added, for instance, to ensure that the regulatory elements of the constant region domain remain free and accessible or that the hinge region remains flexible. Preferably, any linker peptide compatible with the instant invention will be relatively non-immunogenic and not inhibit any non-covalent association of among monomer subunits of an altered binding protein.

In certain embodiments, an altered polypeptide of the invention comprises at least one engineered cysteine residue or analog thereof which is located at the solvent-exposed surface of the polypeptide. Preferably the engineered cysteine residue or analog thereof does not interfere with a biological activity conferred by the constant region or portion thereof of the altered polyeptide. More preferably, the alteration does not interfere with the ability of the altered polyeptide to bind to Fc receptors (e.g. FcγRI, FcγRII, or FcγRIII) or complement proteins (e.g. C1q), or to trigger immune effector function (e.g., antibody-dependent cytotoxicity (ADCC), phagocytosis, or complement-dependent cytotoxicity (CDCC)).

In one embodiment, the engineered cysteine residue or analog thereof is located in a CH1 domain or portion thereof derived from an immunoglobulin heavy chain (e.g. a human IgG1 or IgG4 heavy chain). In another embodiment, the engineered cysteine residue or analog thereof is located in a hinge domain or portion thereof derived from an immunoglobulin heavy chain (e.g. a human IgG1 or IgG4 heavy chain). In another embodiment, the engineered cysteine residue or analog thereof is located in a CH2 domain or portion thereof derived from an immunoglobulin heavy chain (e.g. a human IgG1 or IgG4 heavy chain). In another embodiment, the engineered cysteine residue or analog thereof is located in a CH3 domain or portion thereof derived from an immunoglobulin heavy chain (e.g. a human IgG1 or IgG4 heavy chain). In another embodiment, the engineered cysteine residue or analog thereof is located in a CH4 domain or portion thereof derived from an immunoglobulin heavy chain.

In a particularly preferred embodiment, the altered polypeptides of the invention comprise more than one engineered cysteine residue or analog thereof. The altered polypeptides of the invention may comprise, for example, 2, 3, 4, 5, 6, 7, 8, 9, 10 or more engineered cysteine residues or analogs thereof. Preferably, the engineered cysteines are spatially positioned from each other by an interval of at least 1 amino acid position or more, for example, at least 2, 3, 4, 5, 6, 7, 8, 9, or 10 amino acid positions or more. More preferably, the engineered amino acids are spatially positioned apart from each other by an interval of at least 5, 10, 15, 20, or 25 amino acid positions or more.

In preferred embodiments, the altered polypeptides of the invention contain at least one engineered free cysteine residue or analog thereof that is substantially free of disulfide bonding with a second cysteine residue. Accordingly, in one embodiment, the invention includes a composition comprising a population of the altered polypeptides wherein greater than 50% of the altered polypeptides comprise an engineered free cysteine or analog thereof that is substantially free of disulfide bonding with a second cysteine residue and wherein greater than 50% of the native disulfide bonds are present. In another embodiment, greater than 70% of the altered polypeptides contain an engineered free cysteine that is substantially free of disulfide bonding with a second cysteine residue, and greater than 70% of the native disulfide bonds are present. In another embodiment, greater than 90% of the altered polypeptides contain an engineered free cysteine that is substantially free of disulfide bonding with a second cysteine residue, and greater than 90% of the native disulfide bonds are present.

The altered polypeptides of the invention may be assembled together or with other polypeptides to form altered proteins having two or more polypeptides (ie. “multimers”), wherein at least only polypeptide of the multimer is an altered polypeptide of the invention. The invention is directed without limitation to monomeric as well as multimeric (e.g., dimeric, trimeric, tetrameric, and hexameric) altered proteins and the like. In one embodiment, the polypeptides of the altered protein are the same (ie. homomeric altered proteins, e.g. homodimers, homotetramers). In another embodiment, the polypeptides of the altered protein are different (ie. heteromeric altered proteins, e.g. heterodimers, heterotetramers).

### A. Altered CH3-Containing Polypeptides

In one embodiment of the invention, an altered binding polypeptide comprises at least a portion of an altered CH3 domain wherein the altered CH3 domain comprises at least one engineered cysteine residue or analog thereof (an “altered CH3-containing binding protein”).

The CH3 domain in the Fc region of an immunoglobulin heavy chain is an ideal site for the introduction of a engineered cysteine or analog thereof since it occupies a position in space that is the most distal from the binding site of the immunoglobulin. Alteration of the CH3 domain are therefore unlikely to affect the binding affinity of an antibody or other binding protein comprising an altered CH3 domain. Although other domains in the Fc region, in particular the hinge and CH2 domains, may be selected as candidate alteration sites due to their lack of antigen binding activity, they typically occupy a position in three-dimensional space which is more proximal to the binding site of the binding protein (e.g. the variable region). Alterations in these regions of the Fc region are more likely to interfere with binding affinity. In addition, the hinge and CH2 domains contain residues that are critical for conferring the effector function of the binding protein. Hinge and CH2 domains contain residues which bind to Fc receptors or complement proteins (e.g. C1q) and trigger important effector functions of the antibody. For example, the Fc gamma receptors of immune cells (e.g. FcγRI, FcγRII, or FcγRIII) can activate important cell-mediated immune responses, such as antibody-dependent cytotoxicity (ADCC) or phagocytosis.

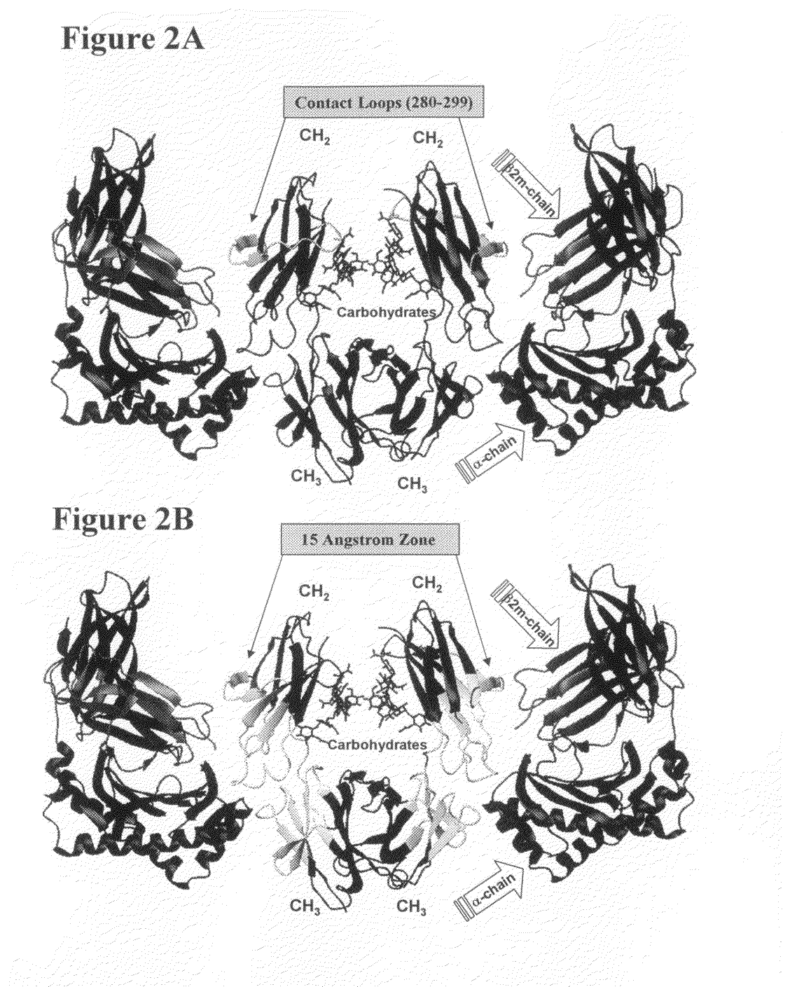

Mutations at, adjacent to, or close to sites in the hinge link region (e.g., replacing residues at EU positions 234, 236 or 237 by Ala) can drastically affect affinity for the monocyte FcγRI receptor. Affinity for other Fc receptors may be altered by similar mutations. Mutations in the C-terminal portion of CH2 region (e.g. at EU positions 318-322) can alter binding to complement protein C1q, leading to reduced recruitment of other complement proteins and the inability to activate complement-dependent cytotoxicity (CDCC). In addition to these antigen-dependent effector functions, binding of CH2 residues to the neonatal receptor (FcRn), is important for regulating the antigen-independent effector functions (e.g. half-life) of the antibody. For example, an important feature for FcRn binding, the FcRn binding loop, is comprised of residues corresponding EU amino acid position 280 to 299, all of which are located in the CH2 domain (see FIG. 2A). Accordingly, cysteine mutations to the FcRn binding loop should be avoided in order to maintain FcRn binding.

While the CH3 domain of the Fc region does not play a critical role in mediating effector function, alteration of residues within this domain can nevertheless result in the perturbation of a desired effector function. Accordingly, in preferred embodiments, altered polypeptides of the invention contain engineered cysteine at amino acid positions the CH3 domain where the original amino acid residue in the unaltered or starting polypeptide is a non-FcR interacting residue. As defined herein, “non-FcR interacting residues” do not participate in modulating the antigen-dependent effector functions (e.g. ADCC, CDC) or antigen-independent effector functions (e.g. half-life) of the starting polypeptide.

In preferred embodiments, altered CH3-containing polypeptides of the invention comprise altered CH3 domains having at least one engineered cysteine at an amino acid position which lies outside a region of the CH3 domain that is known to interact with an Fc receptor. For example, altered polypeptides may comprise engineered cysteines at CH3 amino acid positions that are distant from the Fc/FcR interface. In preferred embodiments, altered polypeptide of the invention include at least one engineered cysteine at an EU amino acid position of the CH3 domain that is not included within the “15 Angstrom Contact Zone” illustrated in FIG. 2B. The 15 Angstrom Zone includes residues located at EU positions 243 to 261, 275 to 280, 282-293, 302 to 319, 336 to 348, 367, 369, 372 to 389, 391, 393, 408, and 424-440 of the Fc region. Accordingly, in preferred embodiments, altered polypeptides of the invention may contain engineered cysteine residues or analogs thereof at any of the following EU positions in the CH3 domain: 349, 350, 351, 352, 353, 354, 355, 356, 357, 358, 359, 360, 361, 362, 363, 364, 365, 366, 368, 370, 371, 390, 392, 394, 395, 396, 397, 398, 399, 400, 401, 402, 403, 404, 405, 406, 407, 409, 410, 411, 412, 413, 414, 415, 416, 417, 418, 419, 420, 421, 422, 423, 441, 442, 443, 444, 445, 446, and 446b. In another embodiment, the altered polypeptide of the invention may comprise an engineered cysteine residue or an analog thereof at the C-terminus of the Fc region. In a more specific embodiment, the altered polypeptide may comprise a substitution of a C-terminal lysine residue at EU position 446b (Kabat position 478).

Particularly preferred altered polypeptides of the invention comprise at least one engineered cysteine residue or analog thereof at an amino acid position wherein the original amino acid at the same amino acid position in the starting polypeptide is on the surface of the molecule and is fully exposed to solvent. More preferably, the altered polypeptides of the invention comprise at least one engineered cysteine or analog thereof at an amino acid position wherein the original amino acid at that position has a cysteine-compatible conformation.

In certain embodiments, the altered polypeptides of the invention comprise at least one engineered cysteine residue or analog thereof at an amino acid position selected from the group consisting of EU position 350, EU position 355, EU position 359, EU position 360, EU position 361, EU position 389, EU position 413, EU position 415, EU position 418, EU position 422, EU position 441, EU position 443, and EU position 446b (Kabat position 478).

In certain preferred embodiments, the altered polypeptides of the invention comprise at least one engineered cysteine residue or analog thereof at an amino acid position selected from the group consisting of EU position 355, EU position 359, EU position 360, EU position 361, EU position 389, EU position 413, EU position 415, EU position 418, EU position 422, and EU position 446b (Kabat position 478). In particularly preferred embodiments, the EU amino acid position is selected from the group consisting of 355, 361, 389, 415, and 446b.

In more specific embodiments, the altered polypeptides of the invention may comprise a substituted amino acid in the CH3 domain of an IgG1 immunoglobulin heavy chain with an engineered cysteine or analog thereof, wherein the original amino acid is selected from the group consisting of a lysine at EU position 350, an arginine at EU position 355, a threonine at EU position 359, a lysine at EU position 360, an asparagine at EU position 361, an asparagine at EU position 389, an aspartic acid at EU position 413, a serine at EU position 415, a glutamine at EU position 418, a valine at EU position 422, a Threonine at EU position 441, a Leucine at EU position 443, and a C-terminal lysine at EU position 446b.

In other specific embodiments, the altered polypeptides of the invention may comprise a substituted amino acid in the CH3 domain of an IgG1 immunoglobulin heavy chain with an engineered cysteine or analog thereof, wherein the original amino acid is selected from the group consisting of an arginine at EU position 355, a threonine at EU position 359, a lysine at EU position 360, an asparagine at EU position 361, an asparagine at EU position 389, an aspartic acid at EU position 413, a serine at EU position 415, a glutamine at EU position 418, a valine at EU position 422, and a C-terminal lysine at EU position 446b. Alternatively, the altered polypeptides of the invention may comprise an engineered cysteine residue or analog thereof at a corresponding homologous amino acid in the CH3 domain of a non-IgG immunoglobulin type (e.g. IgA and IgM).

In other specific embodiments, the altered polypeptides of the invention may comprise a substituted amino acid in the CH3 domain of an IgG4 immunoglobulin heavy chain with an engineered cysteine or analog thereof, wherein the original amino acid is selected from the group consisting of a glutamine at EU position 355, an asparagine at EU position 361, an asparagine at EU position 389, an aspartic acid at EU position 413, a serine at EU position 415, a valine at EU position 422, and a serine at EU position 442.

In yet other specific embodiments, the altered polypeptides of the invention may comprise a substituted amino acid in the CH3 domain of an IgG4 immunoglobulin heavy chain with an engineered cysteine or analog thereof, wherein the original amino acid is selected from the group consisting of a glutamine at EU position 355, an asparagine at EU position 361, an asparagine at EU position 389, and a serine at EU position 442.

In another embodiment, an altered polypeptide of the invention comprises a CH3 domain having the amino acid sequence depicted in FIG. 1E (SEQ ID NO:5) or a portion thereof, wherein at least one amino acid of the amino acid sequence is substituted with an engineered cysteine residue or analog thereof. In one embodiment, the altered polypeptide comprises the CH3 domain depicted in FIG. 1F (SEQ ID NO:6) or a portion thereof, wherein at least one amino acid is substituted with an engineered cysteine residue or analog thereof. In another embodiment, the altered polypeptide comprises the CH3 domain depicted in FIG. 1G (SEQ ID NO:7) or a portion thereof, wherein at least one amino acid is substituted with an engineered cysteine residue or analog thereof.

In a preferred embodiment, the altered polypeptide comprises at least two engineered cysteines where the engineered cysteines are located at least two of the following EU positions 355, 361, 389, and 415. In one embodiment, the altered polypeptide comprises an engineered cysteine residue or analog thereof at EU position 355 and EU position 361. In another embodiment, the altered polypeptide comprises an engineered cysteine residue or analog thereof at EU position 355 and EU position 389. In another embodiment, the altered polypeptide comprises an engineered cysteine residue or analog thereof at EU position 355 and EU position 415. In one embodiment, the altered polypeptide comprises an engineered cysteine residue or analog thereof at EU position 355 and EU position 446b (C-terminal residue). In another embodiment, the altered polypeptide comprises an engineered cysteine residue or analog thereof at EU position 361 and EU position 389. In another embodiment, the altered polypeptide comprises an engineered cysteine residue or analog thereof at EU position 361 and EU position 415. In another embodiment, the altered polypeptide comprises an engineered cysteine residue or analog thereof at EU position 361 and EU position 446b (C-terminal residue). In another embodiment, the altered polypeptide comprises an engineered cysteine residue or analog thereof at EU position 389 and EU position 415. In another embodiment, the altered polypeptide comprises an engineered cysteine residue or analog thereof at EU position 389 and EU position 446b (C-terminal residue). In another embodiment, the altered polypeptide comprises an engineered cysteine residue or analog thereof at EU position 415 and EU position 446b (C-terminal residue). In another embodiment, the engineered cysteines or analogs thereof are located all of EU positions 355, 361, 389, 415, and 446b.

In certain embodiments, the altered polypeptides of the invention are capable of expression by a host cell at a yield of at least 2 mg/L of host cell culture media. More preferably, the altered polypeptides are capable of expression by a host cell at a yield of at least 3, at least 4, at least 5, at least 6, at least 7, at least 8, at least 9, at least 10, at least II, at least 12, at least 15, at least 20, at least 25, at least 50, at least 70, or at least 100 mg/L. In preferred embodiments, the host cell is a mammalian host cell. Exemplary mammalian host cells include Chinese Hamster Ovary (CHO) cells, HELA (human cervical carcinoma) cells, CVI (monkey kidney line) cells, COS (a derivative of CVI with SV40 T antigen) cells, R1610 (Chinese hamster fibroblast) cells, BALBC/3T3 (mouse fibroblast) cells, HAK (hamster kidney line) cells, SP2/O (mouse myeloma) cells, BFA-1c1BPT cells (bovine endothelial cells), RAJI (human lymphocyte) cells and 293 cells (human kidney). In other preferred embodiments, at least 10% (e.g. at least 15%, 20%, 25%, 30%, 40%, 50%, 60%, 70%, 80%, or 90%) of the altered polypeptides of the invention can be purified in an aggregate-free form (e.g. by size exclusion chromatography) following expression in a host cell.

In a particularly preferred embodiment, the altered polypeptide comprises an engineered cysteine residue or analog thereof at one or both of EU positions 355 and 415. Said altered polypeptides have particularly desired expression characteristics. In a preferred embodiment, the altered polypeptide comprising a CH3 domain is capable of expression by a host cell at a yield of at least 5 mg per liter of host cell culture medium. In a still more preferred embodiment, the altered polypeptide is capable of expression by a host cell at a yield of at least 5 mg per liter of host cell culture medium. In a preferred embodiment, the host cell is a mammalian host cell (e.g. a CHO cell).

In certain embodiments, the altered CH₃-containing polypeptides of the invention may further comprise an altered CH2 domain or portion thereof having at least one engineered cysteine residue or analog thereof. Exemplary altered CH2 domains a described in section D infra.

### B. Altered CH1-Containing Polypeptides

In certain embodiments, the altered polypeptides of the invention comprise at least one altered polypeptide comprising at least a portion of an altered CH1 domain having at least one engineered cysteine residue or analog thereof (“altered CH1-containing polypeptides”). In certain embodiments, the altered CH1-containing polypeptide is an altered heavy chain of an immunoglobulin. In other more preferred embodiments, the altered CH1-containing binding polypeptide is an altered constant region domain of the antigen binding fragment of an immunoglobulin.

In another embodiment, an altered polypeptide comprises at least one CH1 domain or portion thereof having at least one engineered cysteine residue or analog thereof at an EU amino acid position where the original amino acid in the corresponding starting or unaltered polypeptide is (1) on the surface of the polypeptide (ie. fully exposed to solvent), and/or (2) has a cysteine-compatible conformation.

In preferred embodiments, the engineered cysteine residue or analog thereof is located at an EU amino acid position selected from the group consisting of 132 (Kabat position 128), 138 (Kabat position 136), 164 (Kabat position 167), and 191 (Kabat position 196). In more specific embodiments, the engineered cysteine residue or analog thereof replaces an amino acid at an EU position in the CH1 domain of a human IgG1, wherein the amino acid is selected from the group consisting of a serine at EU position 132 (Kabat position 128), a glycine at EU position 138 (Kabat position 136), a threonine at EU position 164 (Kabat position 167), and a serine at EU position 191 (Kabat position 196). In another embodiment, an altered CH1 containing polypeptide may comprise an engineered cysteine residue at a corresponding EU position in the CH1 domain of an IgG2, IgG3, or IgG4 immunoglobulin or an immunoglobulin of a non-IgG class (e.g. IgA and IgM).

In certain embodiments, the altered CH1-containing polypeptides of the invention comprise an altered polyeptide with an altered CH1 domain having more than one engineered free cysteine residue or analog thereof. Such altered polypeptides may comprise, for example, 2, 3, or 4 engineered cysteines or analogs thereof. In one embodiment, the altered CH1-containing polypeptide comprises engineered cysteines or analogs thereof at any two EU positions selected from the group consisting of 132 and 138 (Kabat positions 128 and 136), 132 and 164 (Kabat positions 128 and 167), 132 and 191 (Kabat positions 128 and 196), and 138 and 164 (Kabat positions 136 and 167), 138 and 191 (Kabat positions 136 and 196), 164 and 191 (Kabat positions 167 and 196). In another embodiment, the altered CH1-containing polypeptide comprises engineered cysteines or analogs thereof at any three EU positions selected from the group consisting of 132, 138, and 164 (Kabat positions 128, 136, and 167), 138, 164, and 191 (Kabat positions 136, 167, 196), 132, 164, 191 (Kabat positions 128, 167, and 196), and 132, 138 and 191 (Kabat positions 128, 136 and 196). In another embodiment, the altered CH1-containing polypeptide comprises engineered cysteines or analogs thereof at the following four EU positions: 132, 138, 164, and 191 (Kabat positions 128, 136, 164, 196).

In one embodiment, an altered polypeptide of the invention comprises a CH 1 domain (EU positions 118-215) having the amino acid sequence depicted in FIG. 1A (SEQ ID NO: 1) or a portion thereof, wherein at least one amino acid of the amino acid sequence is substituted with an engineered cysteine residue or analog thereof.

In certain embodiments, the altered CH1-containing polypeptides of the invention may further comprise an altered CH3 domain portion having at least one engineered cysteine residue or analog thereof, for example, at any of the amino acid positions supra. In other embodiments, the altered CH1-containing polypeptides of the invention may further comprise an altered CH2 domain or portion thereof having at least one engineered cysteine residue or analog thereof. In other embodiments, the altered CH1 containing polypeptide of the invention may comprise an altered CH2 domain and an altered CH3 domain each of which comprise at least one engineered cysteine residue or analog thereof.

### C. Altered CL-Containing Polypeptides

In another embodiment, the altered polypeptides of the invention comprise at least a portion of an constant light chain (“CL”) domain having at least one engineered cysteine residue or analog thereof (“altered CL-containing polypeptides”). In certain embodiments, the altered CL-containing polypeptide is an altered light chain of an immunoglobulin. In other embodiments, the altered CL-containing polypeptide is an altered light chain portion of an antigen binding fragment of an immunoglobulin.

Particularly preferred altered CL-containing polypeptides comprise at least one CL domain or portion thereof having at least one engineered cysteine residue or analog thereof where the original amino acid at the same amino acid position in the starting or unaltered polypeptide is (1) on the surface of the polypeptide (ie. fully exposed to solvent), and/or (2) has a cysteine-compatible conformation.

In certain exemplary embodiments, the altered CL-containing polypeptide comprises at least one engineered cysteine residue or analog thereof at a Kabat amino acid position selected from the group consisting of 122, 127, 158, and 184. In more specific embodiments, the CL-containing polypeptide comprises at least one engineered cysteine residue or analog thereof that replaces an amino acid at an amino acid position in the CL domain of a human kappa light chain, wherein the amino acid is selected from the group consisting of an aspartate or aspartic acid at Kabat position 122, a serine at Kabat position 127, an asparagine at Kabat position 158, and an alanine at Kabat position 184. In another embodiment, an altered CL-containing polypeptide may comprise an engineered cysteine residue at an amino acid position in the CL domain of a human lambda light chain.

In certain embodiments, the altered CL-containing polypeptides comprise an altered CL domain having more than one engineered free cysteine residue or analog thereof. Such altered polypeptides may comprise, for example, 2, 3, or 4 engineered cysteines or analogs thereof. In one embodiment, the altered CL-containing polypeptide comprises engineered cysteines or analogs thereof at any two Kabat positions selected from the group consisting of 122 and 127, 122 and 158, 122 and 184, 127 and 158, 127, and 184, and 158 and 184. In another embodiment, the altered CL-containing polypeptide comprises engineered cysteines or analogs thereof at three Kabat positions selected from the group consisting of 122, 127, and 158, 122, 127 and 184, 122, 158, and 184, and 127, 158, and 184. In another embodiment, the altered CH1-containing polypeptide comprises engineered cysteines or analogs thereof at the following four Kabat positions: 122, 127, 158, and 184.

### D. Altered CH₂-Containing Polypeptides

In certain embodiments, the altered polypeptides of the invention comprise at least one altered polypeptide comprising at least a portion of an altered CH2 domain having at least one engineered cysteine residue or analog thereof (“altered CH2-containing polypeptides”). In certain embodiments, the altered CH2-containing polypeptide is an altered heavy chain of an immunoglobulin. In other more preferred embodiments, the altered CH2-containing binding polypeptide is an altered constant region domain of the antigen binding fragment of an immunoglobulin.

In another embodiment, an altered polypeptide comprises at least one CH2 domain or portion thereof having at least one engineered cysteine residue or analog thereof at an EU amino acid position where the original amino acid in the corresponding starting or unaltered polypeptide is (1) on the surface of the polypeptide (ie. fully exposed to solvent), and/or (2) has a cysteine-compatible conformation.

In preferred embodiments, the engineered cysteine residue or analog thereof is located at EU amino acid position 274 or 324. In more specific embodiments, the engineered cysteine residue or analog thereof replaces an amino acid at an EU position in the CH2 domain of a human IgG1, wherein the amino acid is a lysine at EU position 274 or a serine at EU position 324.

In certain embodiments, the altered CH₂-containing polypeptides of the invention comprise an altered polyeptide with an altered CH2 domain having more than one engineered free cysteine residues or analogs thereof. Such altered polypeptides may comprise, for example, 2, 3, or 4 engineered cysteines or analogs thereof. In one embodiment, the altered CH2-containing polypeptide comprises engineered cysteines or analogs thereof both EU positions 274 and 324.

In certain embodiments, the altered CH2-containing polypeptides of the invention may further comprise an altered CH3 domain portion having at least one engineered cysteine residue or analog thereof, for example, at any of the amino acid positions supra. In other embodiments, the altered CH2-containing polypeptides of the invention may further comprise an altered CH1 domain or portion thereof having at least one engineered cysteine residue or analog thereof as describe supra. In other embodiments, the altered CH2 containing polypeptide of the invention may comprise an altered CH1 domain and an altered CH3 domain each of which comprise at least one engineered cysteine residue or analog thereof.

### III. DESIGN OF ALTERED POLYPEPTIDES COMPRISING A CONSTANT REGION DOMAIN

The present invention provides methods for identifying alteration sites or candidate amino acid residues in polypeptide that are suitable for the introduction of engineered cysteine residue(s) or analog thereof. The methods of the invention include molecular or computational modeling. Generally, the methods begin with a “first”, “unaltered”, “original” or “starting” polypeptide, or a complex (e.g. crystal structure or homology model) containing it, and result in a “second” or “altered” polypeptide containing at least one engineered cysteine residue or analog thereof which preferably has substantially similar or equivalent activity (e.g. antigen binding affinity and/or effector function) to the starting polypeptide. The modeling can be carried out in silico. Alternatively, the methods may comprise an empirical analysis of which residues that can be altered without appreciable effects on the activity of the starting polypeptide.

The methods employ a general set of selection criteria. One criterion is that the alteration site be present on the surface of the starting polypeptide so that, when altered with an engineered cysteine residue or analog thereof, the engineered cysteine residue is exposed to solvent and is capable of being conjugated, e.g. covalently or noncovalently linked, to an effector moiety. Preferably, the alteration site will be present on the surface of the properly folded or native structure of the polypeptide. A second criterion is that, when altered with an engineered cysteine residue or analog thereof, the alteration site does not abrogate the desired biological function of the starting polypeptide or conversely, that the alteration site does interfere with an unwanted activity of the starting polypeptide. A third criterion is that the alteration site be chosen such the engineered cysteine has a low probability of forming a disulfide bond with a native cysteine residue in the same starting polypeptide or with a cysteine in different polypeptide sequence. A fourth criterion is that the alteration site, when modified with an effector moiety, also does not result in abrogation of a desired activity of the starting polypeptide, or conversely, does result in abrogation of an unwanted activity.

In one embodiment, the mutation does not compromise an existing functionality or biological activity of the starting polypeptide (e.g, antigen, ligand, or receptor binding or an Fc mediated effector function) or diminish from its intended use. Introduced mutations, therefore, preferably maintain the advantages that the Fc region provides. For example, Fc-containing polypeptides often have ADCC functionality. This important cell killing activity would be partially or wholly lost in constructs where the engineered cysteine is introduced at a site that is important for mediating this activity, e.g., sites which mediate interactions with the Fc gamma receptors. Maintaining Fc-dependent ADCC functionality can be important in certain applications because it can elicit a cell killing affect serving to enhance the efficacy of the anti-cancer drug or other drug that works by an ADCC dependent depletion mechanism.

In preferred embodiments, the altered polypeptides of the invention contain mutations that do not reduce other desirable immune effector or receptor binding functions of the starting polypeptide. In particularly preferred embodiments, the altered polypeptides contain mutations that do not alter binding of the altered polypeptide to an FcR that is capable of facilitating purification of the altered polypeptide, in particular Staphylococcal Protein A or G. The site on Fc responsible for binding to Protein A is known in the art (Deisenhofer J. 1981 Biochemistry. April 28; 20(9):2361-70).

The methods may comprise one or more steps. For example, the method may comprise providing a structure of a complex, or data corresponding thereto, between the target polypeptide and an Fc-binding protein (e.g. an FcR). In another or subsequent step, the methods may comprise identifying a defined residue or set of residues (ie. candidate amino acids) within a constant region of a starting polypeptide that can be modified (e.g., mutated) and are predicted to have minor effect on the binding affinity for the Fc-binding protein compared to the starting polypeptide. In other steps, the method may comprise selecting a smaller set of candidate amino acids (ie. elected amino acids) that can be substituted with a engineered cysteine or analog thereof at their position, without appreciable effects on the structural or biochemical properties of the starting polypeptide.

### A. Conformational Analysis

In one embodiment, the methods for identifying the candidate amino acid(s) comprise an analysis (e.g. visual inspection or computational analysis) of a starting polypeptide (e.g., an Fc-containing polypeptide) and/or a starting polypeptide bound to an Fc-binding protein (e.g. an FcR).

The three-dimensional (3-D) structure of a polypeptide influences its biological activity and stability, and that structure can be determined or predicted in a number of ways. Generally, empirical methods use physical biochemical analysis. Alternatively, tertiary structure can be predicted using model building of three-dimensional structures of one or more homologous polypeptides, proteins (or protein complexes) that have a known three-dimensional structure. X-ray crystallography is perhaps the best-known way of determining protein structure (accordingly, the term “crystal structure” may be used in place of the term “structure”) (for example, the crystal structure of the human IgG1 Fc region has been determined (Disenhofer et al., Biochemistry, (1981), 20: 2361-70)), but estimates can also be made using circular dichroism, light scattering, or by measuring the absorption and emission of radiant energy. Other useful techniques include neutron diffraction, nuclear magnetic resonance (NMR), and homology modeling. All of these methods are known to those of ordinary skill in the art, and they have been well described in standard textbooks (see, e.g., Physical Chemistry, 4th Ed., W. J. Moore, Prentiss-Hall, N.J., 1972, or Physical Biochemistry, K. E. Van Holde, Prentiss-Hall, N.J., 1971)) and numerous publications. Any of these techniques can be carried out to determine the structure of a constant region domain (e.g. an Fc region), a polypeptide comprising a constant region domain, or a complex of the polypeptide and a constant region domain binding protein (e.g. Fc-binding protein), which can then be analyzed accordingly to predict amino acids for substitution and/or used to inform one or more steps of a procedure (e.g., such as those described infra).

Methods for forming crystals of starting polypeptides such as antibodies and antibody fragments have been reported by, for example, van den Elsen et al. (Proc. Natl. Acad. Sci. USA 96:13679-13684, 1999, which is expressly incorporated by reference herein). Such art-recognized techniques can also be carried out to determine the structure of a constant region (e.g. Fc region) or a complex containing a constant region polypeptide and a binding protein (e.g. Fc-binding protein) for analysis according to the methods of the present invention. Alternatively, published structures of a constant region (e.g. an Fc region), a constant region-binding protein complex (e.g. Fc/FcR complex), or data corresponding thereto, may be readily available from a commercial or public database, e.g. the Protein Data Bank. Where the structure of a constant region or complex (e.g. an X-ray structure) or data corresponding thereto is not known or available, a homology model using a related constant region or complex (e.g. from another species or a homologous ligand/receptor complex) may be utilized. For example, the crystal structure of the rat Fc-FcRn complex can be used to model the interaction of human Fc with FcRn.

Data corresponding to a polypeptide comprising a constant region domain structure or polypeptide comprising a constant region domain/binding protein complex can be evaluated to determine a potential alteration site, for example, those regions of the starting polypeptide that are solvent-accessible. In another embodiment, the methods comprise an analysis (e.g. structural or computational analysis) of conformational differences between a free (ie. unbound) constant region polypeptide and a polypeptide comprising a constant region domain bound to a binding protein.

In one embodiment, a visual analysis (e.g. using a 3-D molecular visualizer) of a three-dimensional structure and/or model of a target polypeptide or a Fc-FcRn complex thereof can be visually analyzed to predict engineered cysteines that will have negligible effects on the molecular conformation of the starting polypeptide.

In one embodiment the target amino acid can be selected for substitution with an engineered cysteine or analog thereof if it has cysteine-compatible side chain chemistry. In an exemplary embodiment, the target amino acid may be selected for substitution if its size is compatible or similar to a cysteine (e.g. alanine, glycine, serine, aspartate, asparagine, or valine). In exemplary embodiment, the target amino acid may be selected for substitution if its charge is compatible or similar to that of cysteine. Residues with cysteine-compatible charges have side chains with a net zero charge and non-zero partial charges in different portions of their side chains (e.g. methionine, phenylalanine, tryptophan, serine, tyrosine, asparagines, glutamine). These amino acids can participate in similar hydrophobic interactions and electrostatic interactions.

### B. Electrostatic Modeling

In one embodiment, starting polypeptides are altered according to the results of a computational analysis of electrostatic forces between the polypeptide comprising a constant region domain and a constant region-binding protein (e.g. an FcR), preferably, in accordance to the discrete criteria or rules of the invention described herein. The computational analysis allows one to predict the charge distribution within the polypeptide receptor complex, and one way to represent the charge distribution in a computer system is as a set of multipoles. Alternatively, the charge distribution can be represented by a set of point charges located at the positions of the atoms of the polypeptide. Once a charge distribution is determined, one can determine whether the introduction of a free cysteine will alter that charge distribution. The basic computational formulae used in carrying out electrostatic modeling are provided in, e.g., U.S. Pat. No. 6,230,102, the contents of which are hereby incorporated by reference in the present application in their entirety.

The computational analysis can be mediated by a computer-implemented process that carries out the calculations described in U.S. Pat. No. 6,230,102 (or as described in Tidor and Lee, J. Chem. Phys. 106:8681, 1997; Kangas and Tidor, J. Chem. Phys. 109:7522, 1998). The computer program may be adapted to consider the real world context of Fc binding interaction (and unlike other methods, the methods of the invention take into account, e.g., solvent, long-range electrostatics, and dielectric effects in the binding between a polypeptide comprising a constant region domain and constant region-binding protein in a solvent (e.g., an aqueous solvent such as water, phosphate-buffered saline (PBS), plasma, or blood)). The process is used to identify modifications to the polypeptide structure that will achieve a charge distribution on the modified polyeptide that minimizes the electrostatic contribution to binding free energy between the altered polypeptide and binding protein (compared to that of the starting polypeptide). As is typical, the computer system (or device(s)) that performs the operations described here (and in more detail in U.S. Pat. No. 6,230,102) will include an output device that displays information to a user (e.g., a CRT display, an LCD, a printer, a communication device such as a modem, audio output, and the like). In addition, instructions for carrying out the method, in part or in whole, can be conferred to a medium suitable for use in an electronic device for carrying out the instructions. Thus, the methods of the invention are amendable to a high throughput approach comprising software (e.g., computer-readable instructions) and hardware (e.g., computers, robotics, and chips). The computer-implemented process is not limited to a particular computer platform, particular processor, or particular high-level programming language. A useful process is set forth in U.S. Pat. No. 6,230,102 and a more detailed exposition is provided in Lee and Tidor (J. Chem. Phys. 106:8681-8690, 1997); each of which is expressly incorporated herein by reference.

To determine the effects of an engineered cysteine or analog thereof on the binding affinity of an altered constant region polypeptide with a constant region binding protein, basic sequence and/or structural data is first acquired. The electrostatic distribution of the starting polypeptide can also be determined. Using a continuum electrostatics model, an electrostatic charge can be determined for each side chain of the amino acids in the constant region of the polypeptide. Target amino acid residue(s) may then be selected for substitution with an engineered cysteine residue and structural data corresponding to the altered polypeptide is subjected to further computational analysis. Based on these calculations, the binding affinity is then determined for a subset of altered polypeptides having one or more engineered cysteine residues. For example, candidate amino acid side chain positions, and substitution with engineered cysteine or analogs thereof to these positions, are then determined based on the effects on the electrostatic binding free energy. Binding free energy difference (ΔG in kcal/mol) in going from the native residue to a cysteine residue can be calculated. Positive numbers indicate a predicted decrease in binding affinity, while negative numbers indicate a predicted increase in binding affinity. Accordingly, in one embodiment, the target amino acid residue is selected as a candidate for substitution if no appreciable change in the binding free energy of the altered polypeptide is calculated (e.g. +0.3 kcal/mol<ΔG <−0.3 kcal/mol).

As described herein, the altered polypeptides can be built in silico and the binding energy recalculated. Modified side chains can be built by performing a rotamer dihedral scan in CHARMM, using dihedral angle increments of 60 degrees, to determine the most desirable position for each side chain. Binding energies are then calculated for the wild type (starting) and mutant (altered) complexes using the Poisson-Boltzmann electrostatic energy and additional terms for the van der Waals energy and buried surface area. Results from these computational modification calculations are then reevaluated as needed, for example, after subsequent reiterations of the method either in silico or informed by additional experimental structural/functional data.

### C. Side Chain Repacking

In another embodiment, the method for selecting an amino acid for substitution with an engineered cysteine residue or analog thereof comprises the application of sidechain repacking techniques to a structure (e.g. the crystal structure or model) of a complex containing the constant region-containing polypeptide and an constant region-binding polypeptide. In a sidechain repacking calculation, the target residues can be altered to engineered cysteines computationally, and the stability of the resulting altered polypeptides in the conformation bound to the constant-region binding protein is evaluated computationally. The sidechain repacking calculation generates a ranked list of the altered polypeptides that have altered stability (i.e., altered intramolecular energy).

Exemplary computational algorithms used to rank the results of the computational analysis include dead-end elimination and tree search algorithms (see for example, Lasters et al. (Protein Eng. 8:815-822, 1995), Looger and Hellinga (J. Mol. Biol. 307:429-445, 2001), and Dahiyat and Mayo (Protein Sci. 5:895-903, 1996)). Altered polypeptides that lack an appreciable alteration (e.g. increase or decrease) of receptor binding affinity can then be selected for experimental expression.

### D. 3-D Visualization

In another embodiment the target amino acid can be selected for substitution with an engineered cysteine if it is located in region of the starting polypeptide with a cysteine-compatible microenvironment. In exemplary embodiment, the target amino acid may be selected for substitution if the target amino acid and/or adjacent amino acids are in a conformation that is suitable for accommodating a cysteine residue. Target amino acids with suitable stereochemical conformations can be determined by evaluating torsion angles (ie. rotation or dihedral angles) in the side chain of the target amino acid. Side chain torsion or “chi” (χ) angles define the stereoisomer or “rotamer” state or a particular amino acid, while backbone torsion angles about each Cα-N peptide bond (“phi (φ) torsion angle”) or Cα-C bond (“psi (ψ) torsion angle define the local conformation of the polypeptide backbone. Certain rotamers are higher in energy than others because of steric interactions with neighboring atoms. Electrostatic interactions including hydrogen bonds also affect side-chain energies. These interactions can be “backbone-independent”, that is, not depending on the conformation of the local backbone of the residue or “backbone-dependent”, that is, depending on the local backbone conformation as determined by the backbone dihedrals phi and psi.

Whether a particular target residue exhibits a cysteine compatible conformations can be determined by querying a database of known torsion angles from wide variety of protein structures. Suitable databases include, for example, backbone-dependent rotamer libraries which provide average dihedral angles, standard deviations in dihedral angles, and probabilities of protein side-chain conformations for particular amino acids as a function of the observed backbone torsion angles in a protein structure (see, for example, Dunbrack and Karplus, J. Mol. Biol., (1993), 230: 543-74). Preferably, the candidate target residue has side chain torsional angles which occur frequently for the particular backbone conformation. The candidate target residue may be elected for a substitution with an engineered cysteine or analog thereof if the backbone and/or side chain torsion angles have also been observed with cysteine residues. The probability that a particular target residue has a cysteine-compatible conformation is increased if the cysteine residue exhibits similar backbone and side chain torsional angles at the same or similar frequency as the target amino acid.

### E) Sequence Analysis

The target site or residue to be altered can be identified using a structural analysis of the starting polypeptide or its analogs or by sequence homology. For example, sequence analysis of the starting polypeptide can be used to identify an alteration site that is distant from a native cysteine residue present in the starting polypeptide so that the formation of spurious disulfide bonds may be avoided. Preferably, the engineered cysteine residue is spatially positioned from a native cysteine residue by an interval of at least 1 amino acid position or more, for example, 2, 3, 4, 5, 6, 7, 8, 9, 10 or more amino acid positions. A preferred spatial position can be based on the predicted proximity of the engineered cysteine residue to the native cysteine residue as well as the steric and/or charge of the engineered cysteine to be introduced at the alteration site. Alternatively, a determination as to the optimal positioning may be informed by empirical observations after substitutions of cysteine residues at one or more positions and/or using art recognized in silico or computer-based approaches for determining the steric bulk, charge, and/or the distance of the engineered cysteine residue from the native cysteine residue. Amino acid distances of about 1, 2, 3, 4, 5, 6, 7, 8, 9, 10, 10-20 or more, or any interval of the foregoing ranges are within the scope of the invention.

### III. PREPARATION OF ALTERED POLYPEPTIDES

Having designed an altered polypeptide, a variety of methods are available for producing it. Because of the degeneracy of the code, a variety of nucleic acid sequences will encode the amino acid sequence of the altered polypeptide. The desired polynucleotide can be produced by de novo solid-phase DNA synthesis or by PCR mutagenesis of an earlier prepared polynucleotide encoding the target polypeptide.

Oligonucleotide-mediated mutagenesis is one method for preparing a substitution, or in-frame insertion of an alteration (e.g., altered codon) to introduce a codon encoding an engineered cysteine or analog thereof. For example, the starting polypeptide DNA is altered by hybridizing an oligonucleotide encoding the desired mutation to a single-stranded DNA template. After hybridization, a DNA polymerase is used to synthesize an entire second complementary strand of the template that incorporates the oligonucleotide primer. In one embodiment, genetic engineering, e.g., primer-based PCR mutagenesis, is sufficient to incorporate an alteration, as defined herein, for producing a polynucleotide encoding an altered polypeptide of the invention.

Polynucleotide sequence encoding the altered polypeptide can then be inserted in a suitable expression vector and transfected into prokaryotic or eukaryotic host cells such as E. coli cells, simian COS cells, Chinese Hamster Ovary (CHO) cells or myeloma cells that do not otherwise produce said proteins, for recombinant expression. An affinity tag sequence (e.g. a His(6) tag, SEQ ID NO: 59) may optionally be attached or included within the starting polypeptide sequence to facilitate downstream purification. Where the altered polypeptide is part of an antibody, polynucleotides encoding additional light and heavy chain variable regions, optionally linked to constant regions, may be inserted into the same or different expression vector. The DNA segments encoding immunoglobulin chains are the operably linked to control sequences in the expression vector(s) that ensure the expression of immunoglobulin polypeptides.

Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences.

These expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362).

E. coli is one prokaryotic host particularly useful for cloning the polynucleotides (e.g., DNA sequences) of the present invention. Other microbial hosts suitable for use include bacilli, such as Bacillus subtilus, and other enterobacteriaceae, such as Salmonella, Serratia, and various Pseudomonas species.

Other microbes, such as yeast, are also useful for expression. Saccharomyces and Pichia are exemplary yeast hosts, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

In addition to microorganisms, mammalian tissue culture may also be used to express and produce the altered polypeptides of the present invention (e.g., polynucleotides encoding immunoglobulins or fragments thereof). See Winnacker, From Genes to Clones, VCH Publishers, N.Y., N.Y. (1987). Eukaryotic cells are usually preferred, because a number of suitable host cell lines capable of secreting heterologous proteins (e.g., intact immunoglobulins) have been developed in the art, and include CHO cell lines, various Cos cell lines, HeLa cells, 293 cells, myeloma cell lines, transformed B-cells, and hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992).

Alternatively, antibody-coding sequences can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

The vectors containing the polynucleotide sequences of interest (e.g., the altered polypeptide encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Wherein the altered polyeptides of the invention form multimeric proteins (e.g. antibodies), the multimeric proteins can be expressed using a single vector or two vectors. When the altered polypeptides are cloned on separate expression vectors, the vectors are co-transfected to obtain expression and assembly of intact whole proteins (e.g. antibodies). Once expressed, the whole proteins, their dimers, individual polypeptides (e.g. light and heavy chains), or other forms can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure proteins of at least about 90 to 95% homogeneity are preferred, and 98 to 99% or more homogeneity most preferred, for pharmaceutical uses.

Altered polypeptides should be stored or incubated under appropriate conditions to prevent oxidation or blockage of engineered free cysteines, e.g. by including low concentrations of reducing agent in the purification media. Where engineered free cysteines become blocked or oxidized, the altered polypeptide can be treated with suitable reducing agents (e.g. mercaptoethylamine (MEA)) under conditions which are suitably mild to prevent the reduction of native disulfide bonds within the protein.

### IV. ALTERED BINDING POLYPEPTIDES

In certain aspects, the invention is further directed to altered binding polypeptides comprising an altered polypeptide of the invention together with at least one binding site. Accordingly, the altered binding polypeptides of the invention typically comprise at least one binding site and at least one constant region or portion thereof having at least one engineered cysteine or analog thereof. In certain preferred embodiments, the engineered cysteine residue or analog thereof is located on the solvent-exposed surface of the binding polypeptide. In other preferred embodiments, the engineered cysteine residue or analog thereof is located at a position in three-dimensional space that is distant from a binding site of the binding protein. For example, the engineered cysteine or analog thereof may be located in a non-binding domain of the binding polypeptide or at the C-terminus or N-terminus of a binding domain of the binding polypeptide.

Preferred altered binding polypeptides of the invention comprise at least one of an antigen binding domain, a receptor binding domain, or a ligand binding domain. In one embodiment, the altered binding polypeptide comprises two binding domains and two altered constant regions or portions thereof.

In one embodiment, the altered binding polypeptides of the invention have at least one binding domain specific for a target molecule which mediates a biological effect (e.g., a ligand capable of binding to a cell surface receptor or a cell surface receptor capable of binding a ligand) and mediating transmission of a negative or positive signal to a cell together with at least one altered constant region domain or portion thereof. In one embodiment, the altered binding polypeptides of the invention have at least one binding domain specific for an antigen targeted for reduction or elimination, e.g., a cell surface antigen or a soluble antigen, together with at least one altered constant region domain, e.g. an altered Fc region or FcRn binding portion thereof.

In another embodiment, binding of altered binding polypeptide of the invention to a target molecule (e.g. antigen) results in the reduction or elimination of the target molecule, e.g., from a tissue or from the circulation. In another embodiment, the altered binding polypeptide has at least one binding domain specific for a target molecule that can be used to detect the presence of the target molecule (e.g., to detect a contaminant or diagnose a condition or disorder). In yet another embodiment, an altered binding polypeptide of the invention comprises at least one binding domain that targets the molecule to a specific site in a subject (e.g., to a tumor cell or blood clot).

In certain embodiments, the altered binding polypeptides of the invention may comprise two or more binding sites. In one embodiment, the binding sites are identical. In another embodiment, the binding sites are different.

In other embodiments, the altered binding polypeptides of the invention may be assembled together or with other polypeptides to form altered binding proteins having two or more polypeptides (“altered binding proteins”) to form multimers, wherein at least one polypeptide of the multimer is an altered binding polypeptide of the invention. Exemplary multimeric forms include dimeric, trimeric, tetrameric, and hexameric altered binding proteins and the like. In one embodiment, the polypeptides of the altered binding protein are the same (ie. homomeric altered binding proteins, e.g. homodimers, homotetramers). In another embodiment, the polypeptides of the altered binding protein are different (e.g. heteromeric).

A. Altered Antibodies or Portions Thereof

In certain embodiments, an altered binding protein of the invention is an altered antibody. Altered antibodies of the invention comprise at least one altered binding polypeptide comprising i) a variable region or portion thereof (e.g. a VL and/or VH domain) and ii) an altered constant region domain having an engineered cysteine or analog thereof. Sequences encoding variable regions may be derived from any immunoglobulin using art recognized protocols. For example, the variable domain may be derived from immunoglobulin produced in a non-human mammal, e.g., murine, guinea pig, primate, rabbit or rat, by immunizing the mammal with the antigen or a fragment thereof. See Harlow & Lane, supra, incorporated by reference for all purposes. The immunoglobulin may be generated by multiple subcutaneous or intraperitoneal injections of the relevant antigen (e.g., purified tumor associated antigens or cells or cellular extracts comprising such antigens) and an adjuvant. This immunization typically elicits an immune response that comprises production of antigen-reactive antibodies from activated splenocytes or lymphocytes.

While the variable region may be derived from polyclonal antibodies harvested from the serum of an immunized mammal, it is often desirable to isolate individual lymphocytes from the spleen, lymph nodes or peripheral blood to provide homogenous preparations of monoclonal antibodies (MAbs) from which the desired variable region is derived. Rabbits or guinea pigs are typically used for making polyclonal antibodies. Mice are typically used for making monoclonal antibodies. Monoclonal antibodies can be prepared against a fragment by injecting an antigen fragment into a mouse, preparing “hybridomas” and screening the hybridomas for an antibody that specifically binds to the antigen. In this well known process (Kohler et al., (1975), Nature, 256:495) the relatively short-lived, or mortal, lymphocytes from the mouse which has been injected with the antigen are fused with an immortal tumor cell line (e.g. a myeloma cell line), thus, producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody. They produce antibodies which are homogeneous against a desired antigen and, in reference to their pure genetic parentage, are termed “monoclonal”.

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of monoclonal antibodies against the desired antigen. Preferably, the binding specificity of the monoclonal antibodies produced by hybridoma cells is determined by immunoprecipitation or by an in vitro assay, such as a radioimmunoassay (RIA) or enzyme-linked immunoabsorbent assay (ELISA). After hybridoma cells are identified that produce antibodies of the desired specificity, affinity and/or activity, the clones may be subcloned by limiting dilution procedures and grown by standard methods (Goding, Monoclonal Antibodies. Principles and Practice, pp 59-103 (Academic Press, 1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

Optionally, antibodies may be screened for binding to a specific region or desired fragment of the antigen without binding to other nonoverlapping fragments of the antigen. The latter screening can be accomplished by determining binding of an antibody to a collection of deletion mutants of the antigen and determining which deletion mutants bind to the antibody. Binding can be assessed, for example, by Western blot or ELISA. The smallest fragment to show specific binding to the antibody defines the epitope of the antibody. Alternatively, epitope specificity can be determined by a competition assay is which a test and reference antibody compete for binding to the antigen. If the test and reference antibodies compete, then they bind to the same epitope or epitopes sufficiently proximal such that binding of one antibody interferes with binding of the other.

DNA encoding the desired monoclonal antibody may be readily isolated and sequenced using any of the conventional procedures described supra for the isolation of constant region domain sequences (e.g., by using oligonucleotide probes that are capable of binding specifically to genes encoding the heavy and light chains of murine antibodies). The isolated and subcloned hybridoma cells serve as a preferred source of such DNA. More particularly, the isolated DNA (which may be synthetic as described herein) may be used to clone the desired variable region sequences for incorporation in the altered antibodies of the invention.

In other embodiments, the binding domain is derived from a fully human antibody. Human or substantially human antibodies may be generated in transgenic animals (e.g., mice) that are incapable of endogenous immunoglobulin production (see e.g., U.S. Pat. Nos. 6,075,181, 5,939,598, 5,591,669 and 5,589,369, each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array to such germ line mutant mice will result in the production of human antibodies upon antigen challenge. Another preferred means of generating human antibodies using SCID mice is disclosed in U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology, 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

In another embodiment, lymphocytes can be selected by micromanipulation and the variable genes isolated. For example, peripheral blood mononuclear cells can be isolated from an immunized mammal and cultured for about 7 days in vitro. The cultures can be screened for specific IgGs that meet the screening criteria. Cells from positive wells can be isolated. Individual Ig-producing B cells can be isolated by FACS or by identifying them in a complement-mediated hemolytic plaque assay. Ig-producing B cells can be micromanipulated into a tube and the VH and VL genes can be amplified using, e.g., RT-PCR. The VH and VL genes can be cloned into an antibody expression vector and transfected into cells (e.g., eukaryotic or prokaryotic cells) for expression.

Alternatively, variable (V) domains can be obtained from libraries of variable gene sequences from an animal of choice. Libraries expressing random combinations of domains, e.g., V_{H} and V_{L} domains, can be screened with a desired antigen to identify elements which have desired binding characteristics. Methods of such screening are well known in the art. For example, antibody gene repertoires can be cloned into a λ bacteriophage expression vector (Huse, W D et al. (1989). Science, 2476:1275). In addition, cells (Francisco et al. (1994), PNAS, 90:10444; Georgiou et al. (1997), Nat. Biotech., 15:29; Boder and Wittrup (1997) Nat. Biotechnol. 15:553; Boder et al. (2000), PNAS, 97:10701; Daugtherty, P. et al. (2000) J. Immunol. Methods. 243:211) or viruses (e.g., Hoogenboom, H R. (1998), Immunotechnology 4:1; Winter et al. (1994). Annu. Rev. Immunol. 12:433; Griffiths, A D. (1998). Curr. Opin. Biotechnol. 9:102) expressing antibodies on their surface can be screened.

Those skilled in the art will also appreciate that DNA encoding antibody variable domains may also be derived from antibody phage libraries, e.g., using pd phage or Fd phagemid technology. Exemplary methods are set forth, for example, in EP 368 684 B1; U.S. Pat. No. 5,969,108; Hoogenboom et al., (2000) Immunol. Today 21:371; Nagy et al. (2002) Nat. Med. 8:801; Huie et al. (2001), PNAS, 98:2682; Lui et al. (2002), J. Mol. Biol. 315:1063, each of which is incorporated herein by reference. Several publications (e.g., Marks et al. (1992), Bio/Technology 10:779-783) have described the production of high affinity human antibodies by chain shuffling, as well as combinatorial infection and in vivo recombination as a strategy for constructing large phage libraries. In another embodiment, ribosomal display can be used to replace bacteriophage as the display platform (see, e.g., Hanes, et al. (1998), PNAS 95:14130; Hanes and Pluckthun. (1999), Curr. Top. Microbiol. Immunol. 243:107; He and Taussig. (1997), Nuc. Acids Res., 25:5132; Hanes et al. (2000), Nat. Biotechnol. 18:1287; Wilson et al. (2001), PNAS, 98:3750; or Irving et al. (2001) J. Immunol. Methods 248:31).

Preferred libraries for screening are human variable gene libraries. V_{L} and V_{H} domains from a non-human source may also be used. Libraries can be naïve, from immunized subjects, or semi-synthetic (Hoogenboom and Winter. (1992). J. Mol. Biol. 227:381; Griffiths et al. (1995) EMBO J. 13:3245; de Kruif et al. (1995). J. Mol. Biol. 248:97; Barbas et al. (1992), PNAS, 89:4457). In one embodiment, mutations can be made to immunoglobulin domains to create a library of nucleic acid molecules having greater heterogeneity (Thompson et al. (1996), J. Mol. Biol. 256:77; Lamminmaki et al. (1999), J. Mol. Biol. 291:589; Caldwell and Joyce. (1992), PCR Methods Appl. 2:28; Caldwell and Joyce. (1994), PCR Methods Appl. 3:S136). Standard screening procedures can be used to select high affinity variants. In another embodiment, changes to V_{H} and V_{L} sequences can be made to increase antibody avidity, e.g., using information obtained from crystal structures using techniques known in the art.

Moreover, variable region sequences useful for producing the altered antibodies of the present invention may be obtained from a number of different sources. For example, as discussed above, a variety of human gene sequences are available in the form of publicly accessible deposits. Many sequences of antibodies and antibody-encoding genes have been published and suitable variable region sequences (e.g. VL and VH sequences) can be chemically synthesized from these sequences using art recognized techniques.

In another embodiment, at least one variable region domain of an altered antibody of the invention is catalytic (Shokat and Schultz. (1990). Annu. Rev. Immunol. 8:335). Variable region domains with catalytic binding specificities can be made using art recognized techniques (see, e.g., U.S. Pat. No. 6,590,080, U.S. Pat. No. 5,658,753). Catalytic binding specificities can work by a number of basic mechanisms similar to those identified for enzymes to stabilize the transition state, thereby reducing the free energy of activation. For example, general acid and base residues can be optimally positioned for participation in catalysis within catalytic active sites; covalent enzyme-substrate intermediates can be formed; catalytic antibodies can also be in proper orientation for reaction and increase the effective concentration of reactants by at least seven orders of magnitude (Fersht et al., (1968), J. Am. Chem. Soc. 90:5833) and thereby greatly reduce the entropy of a chemical reaction. Finally, catalytic antibodies can convert the energy obtained upon substrate binding to distort the reaction towards a structure resembling the transition state.

Acid or base residues can be brought into the antigen binding site by using a complementary charged molecule as an immunogen. This technique has proved successful for elicitation of antibodies with a hapten containing a positively-charged ammonium ion (Shokat, et al., (1988), Chem. Int. Ed. Engl. 27:269-271). In another approach, antibodies can be elicited to stable compounds that resemble the size, shape, and charge of the transition state of a desired reaction (i.e., transition state analogs). See U.S. Pat. No. 4,792,446 and U.S. Pat. No. 4,963,355 which describe the use of transition state analogues to immunize animals and the production of catalytic antibodies. Both of these patents are hereby incorporated by reference. Such molecules can be administered as part of an immunoconjugate, e.g., with an immunogenic carrier molecule, such as KLH.

In another embodiment, a variable region domain of an altered antibody of the invention consists of a V_{H} domain, e.g., derived from camelids, which is stable in the absence of a V_{L} chain (Hamers-Casterman et al. (1993). Nature, 363:446; Desmyter et al. (1996). Nat. Struct. Biol. 3: 803; Decanniere et al. (1999). Structure, 7:361; Davies et al. (1996). Protein Eng., 9:531; Kortt et al. (1995). J. Protein Chem., 14:167).

Further, an altered antibody of the invention may be a fully murine, fully human, chimeric, humanized, non-human primate or primatized antibody. Non-human antibodies, or fragments or domains thereof, can be altered to reduce their immunogenicity using art recognized techniques. Humanized antibodies are antibodies derived from a non-human protein, that retains or substantially retains the properties of the parent antibody, but which is less immunogenic in humans. In the case of humanized target antibodies, this may be achieved by various methods, including (a) grafting the entire non-human variable domains onto human constant regions to generate chimeric target antibodies; (b) grafting at least a part of one or more of the non-human complementarity determining regions (CDRs) into a human framework and constant regions with or without retention of critical framework residues; (c) transplanting the entire non-human variable domains, but “cloaking” them with a human-like section by replacement of surface residues. Such methods are disclosed in Morrison et al., (1984), PNAS. 81: 6851-5; Morrison et al., (1988), Adv. Immunol. 44: 65-92; Verhoeyen et al., (1988), Science 239: 1534-1536; Padlan, (1991), Molec. Immun. 28: 489-498; Padlan, (1994), Molec. Immun. 31: 169-217; and U.S. Pat. Nos. 5,585,089, 5,693,761 and 5,693,762 all of which are hereby incorporated by reference in their entirety.

De-immunization can also be used to decrease the immunogenicity of an altered antibody, or the unaltered antibody from which its binding domains(s) are derived. As used herein, the term “de-immunization” includes modification of T cell epitopes (see, e.g., WO9852976A1, WO0034317A2). For example, VH and VL sequences are analyzed and a human T cell epitope “map” from each V region showing the location of epitopes in relation to complementarity-determining regions (CDRs) and other key residues within the sequence is generated. Individual T cell epitopes from the T cell epitope map are analyzed in order to identify alternative amino acid substitutions with a low risk of altering the activity of the final antibody. A range of alternative VH and VL sequences are designed comprising combinations of amino acid substitutions and these sequences are subsequently incorporated into a range of polypeptides of the invention that are tested for function. Typically, between 12 and 24 variant antibodies are generated and tested. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

In one embodiment, an altered binding protein of the invention is a chimeric antibody. In the context of the present application the term “chimeric antibodies” will be held to mean any antibody wherein at least one binding site (e.g. variable region sequence(s)) is obtained or derived from a first species and the altered constant region is obtained from a second species. In preferred embodiments the binding domain will be from a non-human source (e.g. mouse) and the altered constant region is human.

Preferably, the variable domains in both the heavy and light chains of an altered antibody are altered by at least partial replacement of one or more CDRs and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. It may not be necessary to replace all of the CDRs with the complete CDRs from the donor variable region to transfer the antigen binding capacity of one variable domain to another. Rather, it may only be necessary to transfer those residues that are necessary to maintain the activity of the binding domain. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

In one embodiment, an altered antibody of the present invention comprises at least one CDR from an antibody that recognizes a desired target. In another embodiment, an altered antibody of the present invention comprises at least two CDRs from an antibody that recognizes a desired target. In another embodiment, an altered antibody of the present invention comprises at least three CDRs from an antibody that recognizes a desired target. In another embodiment, an altered antibody of the present invention comprises at least four CDRs from an antibody that recognizes a desired target. In another embodiment, an altered antibody of the present invention comprises at least five CDRs from an antibody that recognizes a desired target. In another embodiment, an altered antibody of the present invention comprises at least six CDRs from an antibody that recognizes a desired target.

In another embodiment, e.g., when a starting antibody that binds to a target of interest is a non-human antibody, a humanized antibody comprising a variable region derived from the starting antibody and one or more human framework regions can be made and altered according to the methods of the invention.

In one embodiment, the altered antibodies of the present invention may be immunoreactive with one or more tumor-associated antigens. For example, for treating a cancer or neoplasia an antigen binding domain of an altered antibody preferably binds to a selected tumor associated antigen. Given the number of reported antigens associated with neoplasias, and the number of related antibodies, those skilled in the art will appreciate that an altered antibody of the invention may comprise a variable region sequence or portion thereof derived from any one of a number of whole antibodies. More generally, such a variable region sequence may be obtained or derived from any antibody (including those previously reported in the literature) that reacts with an antigen or marker associated with the selected condition. Exemplary tumor-associated antigens bound by the altered antibodies of the invention include for example, pan B antigens (e.g. CD20 found on the surface of both malignant and non-malignant B cells such as those in non-Hodgkin's lymphoma) and pan T cell antigens (e.g. CD2, CD3, CD5, CD6, CD7). Other exemplary tumor associated antigens comprise but are not limited to MAGE-1, MAGE-3, MUC-1, HPV 16, HPV E6 & E7, TAG-72, CEA, α-Lewis^{y}, L6-Antigen, CD19, CD22, CD25, CD30, CD33, CD37, CD44, CD52, CD56, mesothelin, PSMA, HLA-DR, EGF Receptor, VEGF Receptor, and HER2Receptor.

In other embodiments, the altered antibodies of the invention may comprise variable region sequences from antibodies which have previously been reported to react with tumor-associated antigens. Exemplary antibodies capable of reacting with tumor-associated antigens include: 2B8, Lym 1, Lym 2, LL2, Her2, B1, BR96, MB1, BH3, B4, B72.3, 5E8, B3F6, 5E10, α-CD33, α-CanAg, α-CD56, α-CD44v6, α-Lewis, and α-CD30. More specifically, these exemplary antibodies include, but are not limited to 2B8 and C2B8 (Zevalin® and Rituxan®, IDEC Pharmaceuticals Corp., San Diego), Lym 1 and Lym 2 (Techniclone), LL2 (Immunomedics Corp., New Jersey), Trastuzumab (Herceptin®, Genentech Inc., South San Francisco), Tositumomab (Bexxar®, Coulter Pharm., San Francisco), Alemtzumab (Campath®, Millennium Pharmaceuticals, Cambridge), Gemtuzumab ozogamicin (Mylotarg®, Wyeth-Ayerst, Philadelphia), Cetuximab (Erbitux®, Imclone Systems, New York), Bevacizumab (Avastin®, Genentech Inc., South San Francisco), BR96, BL22, LMB9, LMB2, MB1, BH3, B4, B72.3 (Cytogen Corp.), SS1 (NeoPharm), CC49 (National Cancer Institute), Cantuzumab mertansine (ImmunoGen, Cambridge), MNL 2704 (Milleneum Pharmaceuticals, Cambridge), Bivatuzumab mertansine (Boehringer Ingelheim, Germany), Trastuzumab-DM1 (Genentech, South San Francisco), My9-6-DM1 (ImmunoGen, Cabridge), SGN-10, -15, -25, and -35 (Seattle Genetics, Seattle), and 5E 10 (University of Iowa). In preferred embodiments, the altered antibodies of the present invention will bind to the same tumor-associated antigens as the antibodies enumerated immediately above. In particularly preferred embodiments, the polypeptides will be derived from or bind the same antigens as Y2B8, C2B8, CC49 and C5E10 and, even more preferably, will comprise domain deleted antibodies (i.e., ΔCH2 antibodies).

In a first preferred embodiment, the altered antibody will bind to the same tumor-associated antigen as Rituxan®. Rituxan® (also known as, rituximab, IDEC-C2B8 and C2B8) was the first FDA-approved monoclonal antibody for treatment of human B-cell lymphoma (see U.S. Pat. Nos. 5,843,439; 5,776,456 and 5,736,137 each of which is incorporated herein by reference). Y2B8 (90Y labeled 2B8; Zevalin®; ibritumomab tiuxetan) is the murine starting of C2B8. Rituxan® is a chimeric, anti-CD20 monoclonal antibody which is growth inhibitory and reportedly sensitizes certain lymphoma cell lines for apoptosis by chemotherapeutic agents in vitro. The antibody efficiently binds human complement, has strong FcR binding, and can effectively kill human lymphocytes in vitro via both complement dependent (CDC) and antibody-dependent (ADCC) mechanisms (Reff et al., Blood 83: 435-445 (1994)). Those skilled in the art will appreciate that engineered cysteine variants of C2B8 or 2B8 may be conjugated with effector moieties according to the methods of the invention, in order to provide modified antibodies that are even more effective in treating patients presenting with CD20+ malignancies.

In other preferred embodiments of the present invention, the altered antibody of the invention will bind to the same tumor-associated antigen as CC49. CC49 binds human tumor-associated antigen TAG-72 which is associated with the surface of certain tumor cells of human origin, specifically the LS174T tumor cell line. LS174T [American Type Culture Collection (herein ATCC) No. CL 188] is a variant of the LS180 (ATCC No. CL 187) colon adenocarcinoma line.

It will further be appreciated that numerous murine monoclonal antibodies have been developed which have binding specificity for TAG-72. One of these monoclonal antibodies, designated B72.3, is a murine IgG1 produced by hybridoma B72.3 (ATCC No. HB-8108). B72.3 is a first generation monoclonal antibody developed using a human breast carcinoma extract as the immunogen (see Colcher et al., Proc. Natl. Acad. Sci. (USA), 78:3199-3203 (1981); and U.S. Pat. Nos. 4,522,918 and 4,612,282 each of which is incorporated herein by reference). Other monoclonal antibodies directed against TAG-72 are designated “CC” (for colon cancer). As described by Schlom et al. (U.S. Pat. No. 5,512,443 which is incorporated herein by reference) CC monoclonal antibodies are a family of second generation murine monoclonal antibodies that were prepared using TAG-72 purified with B72.3. Because of their relatively good binding affinities to TAG-72, the following CC antibodies have been deposited at the ATCC, with restricted access having been requested: CC49 (ATCC No. HB 9459); CC 83 (ATCC No. HB 9453); CC46 (ATCC No. HB 9458); CC92 (ATTCC No. HB 9454); CC30 (ATCC No. HB 9457); CC11 (ATCC No. 9455); and CC15 (ATCC No. HB 9460). U.S. Pat. No. 5,512,443 further teaches that the disclosed antibodies may be altered into their chimeric form by substituting, e.g., human constant regions (Fc) domains for mouse constant regions by recombinant DNA techniques known in the art. Besides disclosing murine and chimeric anti-TAG-72 antibodies, Schlom et al. have also produced variants of a humanized CC49 antibody as disclosed in PCT/US99/25552 and single chain constructs as disclosed in U.S. Pat. No. 5,892,019 each of which is also incorporated herein by reference. Those skilled in the art will appreciate that each of the foregoing antibodies, constructs or recombinants, and variations thereof, may be synthetic and used to provide binding domains for the production of altered antibodies in accordance with the present invention.

In addition to the anti-TAG-72 antibodies discussed above, various groups have also reported the construction and partial characterization of domain-deleted CC49 and B72.3 antibodies (e.g., Calvo et al. Cancer Biotherapy, 8(1):95-109 (1993), Slavin-Chiorini et al. Int. J. Cancer 53:97-103 (1993) and Slavin-Chiorini et al. Cancer. Res. 55:5957-5967 (1995).

In one embodiment, an altered antibody of the invention binds to the CD23 antigen (U.S. Pat. No. 6,011,138). In a preferred embodiment, an altered antibody of the invention binds to the same epitope as the 5E8 antibody. In another embodiment, an altered antibody of the invention comprises at least one CDR from an anti-CD23 antibody, e.g., the 5E8 antibody.

In a preferred embodiment, an altered antibody of the invention binds to the CRIPTO-I antigen (WO02/088170A2 or WO03/083041A2). In a more preferred embodiment, an altered antibody of the invention binds to the same epitope as the B3F6 antibody. In still another embodiment, an altered antibody of the invention comprises at least one CDR from an anti-CRIPTO-I antibody, e.g., the B3F6 antibody.

In another embodiment, an altered antibody of the invention binds to antigen which is a member of the TNF family of receptors (“TNFRs”). In a more specific embodiment, an altered antibody of the invention binds to a TNF receptor family member containing a death domain. In yet a more specific embodiment, the TNF receptor family member is selected from the group consisting of: TNFR1, Fas-R, DR-3, TRAIL-R1, TRAIL-R2, and DR6.

In another embodiment, an altered antibody of the invention binds to a TNF receptor family member lacking a death domain. In one embodiment, the TNF receptor lacking a death domain is involved in tissue differentiation. In a more specific embodiment, the TNF receptor involved in tissue differentiation is selected from the group consisting of LTβR, RANK, EDAR1, XEDAR, Fn14, Troy/Trade, and NGFR. In another embodiment, the TNF receptor lacking a death domain is involved in immune regulation. In a more specific embodiment, TNF receptor family member involved in immune regulation is selected from the group consisting of TNFR2, HVEM, CD27, CD30, CD40, 4-1BB, OX40, and GITR.

In another embodiment, an altered antibody of the invention binds a TNF ligand belonging to the TNF ligand superfamily. In another embodiment, the starting polypeptide binds to a TNF ligand selected from the group consisting of TNF-alpha, LT-alpha, FasL, APO-3L, TRAIL, RANKL, EDAR1 ligand, XEDAR ligand, Fn14 ligand, Troy/Trade ligand, NGF-β, NGF-2/NTF3, NTF5, BDNF, IFRD1, HVEM ligand, CD27L, CD30L, CD40L, 4-1BB-L, OX40L, GITRL, and BAFF.

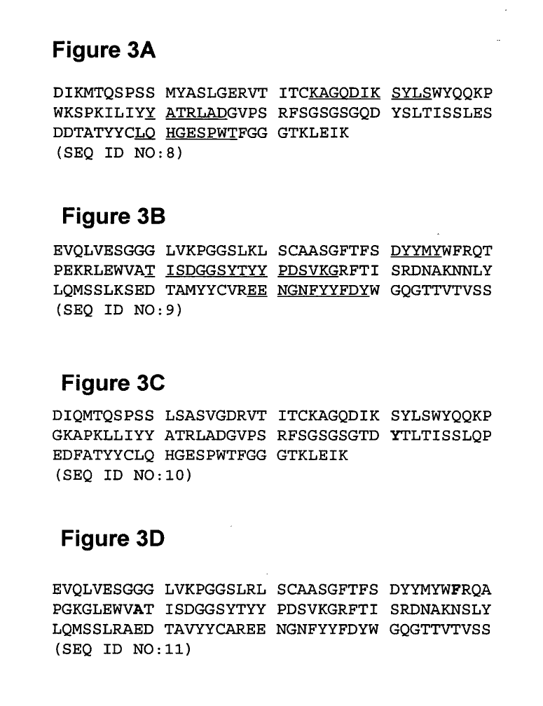

In a more preferred embodiment, an altered antibody of the invention binds to the same epitope as an anti-LTβR antibody (e.g. a CBE11 antibody) set forth in WO 02/30986, which is incorporated herein by reference. In still another embodiment, an altered antibody of the invention comprises at least one CDR from an anti-LTβR antibody, e.g., the CBE11 antibody. In one preferred embodiment, the altered antibody of the invention is a chimeric CBE11 antibody having the mouse light chain variable region sequence depicted in FIG. 3A (SEQ ID NO:8) and the mouse heavy chain variable region sequence depicted in FIG. 3B (SEQ ID NO:9). In another preferred embodiment, the altered antibody of the invention is a humanized CBE11 antibody having the humanized light chain variable region sequence depicted in FIG. 3C (SEQ ID NO: 10) and the humanized heavy chain variable region sequence depicted in FIG. 3D (SEQ ID NO:11).

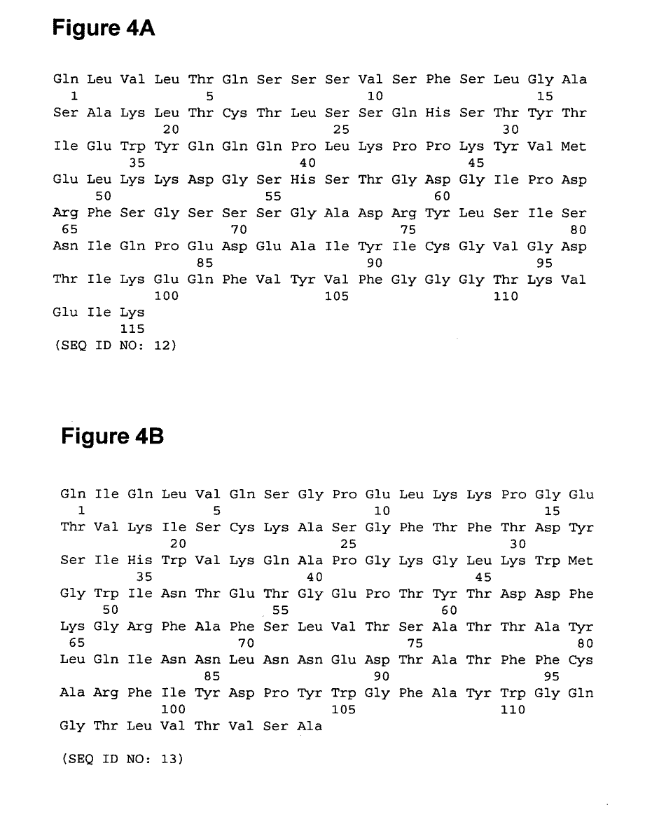

In another preferred embodiment, an altered antibody of the invention binds to the same epitope as an anti-TRAIL-R2 antibody (e.g. a 14A2 antibody). In still another embodiment, a polypeptide of the invention comprises at least one CDR from an anti-TRAIL-R2 antibody, e.g., the 14A2 antibody. In a preferred embodiment, the altered antibody of the invention is a chimeric 14A2 antibody having the mouse light chain variable region sequence depicted in FIG. 4A (SEQ ID NO:12) and the mouse heavy chain variable region sequence depicted in FIG. 4B (SEQ ID NO:13).

Still other embodiments of the present invention comprise altered antibodies that are derived from or bind to the same tumor associated antigen as C5E10. As set forth in co-pending application Ser. No. 09/104,717, C5E10 is an antibody that recognizes a glycoprotein determinant of approximately 115 kDa that appears to be specific to prostate tumor cell lines (e.g. DU145, PC3, or ND1). Thus, in conjunction with the present invention, polypeptides that specifically bind to the same tumor-associated antigen recognized by C5E10 antibodies could be used alone or conjugated with an effector moiety by the methods of the invention, thereby providing a modified polypeptide that is useful for the improved treatment of neoplastic disorders. In particularly preferred embodiments, the starting polypeptide will be derived or comprise all or part of the antigen binding region of the C5E10 antibody as secreted from the hybridoma cell line having ATCC accession No. PTA-865. The resulting polypeptide could then be conjugated to a therapeutic effector moiety as described below and administered to a patient suffering from prostate cancer in accordance with the methods herein.

B. Altered Antibody Variants

In another aspect, the altered binding proteins of the invention may comprise variants of the altered antibodies discussed supra (“altered antibody variants”). Exemplary constructs include, e.g., minibodies, diabodies, diabodies fused to CH₃ molecules, tetravalent antibodies, intradiabodies (e.g., Jendreyko et al. 2003. J. Biol. Chem. 278:47813), fusion proteins (e.g., antibody cytokine fusion proteins, proteins fused to at least a portion of an Fc receptor), and bispecific antibodies. Other immunoglobulins (Ig) and certain variants thereof are described, for example in U.S. Pat. No. 4,745,055; EP 256,654; Faulkner et al., Nature 298:286 (1982); EP 120,694; EP 125,023; Morrison, J. Immun. 123:793 (1979); Kohler et al., Proc. Natl. Acad. Sci. USA 77:2197 (1980); Raso et al., Cancer Res. 41:2073 (1981); Morrison et al., Ann. Rev. Immunol. 2:239 (1984); Morrison, Science 229:1202 (1985); Morrison et al., Proc. Natl. Acad. Sci. USA 81:6851 (1984); EP 255,694; EP 266,663; and WO 88/03559. Reassorted immunoglobulin chains also are known. See, for example, U.S. Pat. No. 4,444,878; WO 88/03565; and EP 68,763 and references cited therein.

In certain embodiments, an altered antibody variant of the invention may comprise an antigen binding fragment of an altered antibody of the invention. The term “antigen-binding fragment” refers to a polypeptide fragment of an immunoglobulin or antibody which binds antigen or competes with intact antibody (i.e., with the intact antibody from which they were derived) for antigen binding (i.e., specific binding). Antigen binding fragments can be produced by recombinant or biochemical methods that are well known in the art. In exemplary embodiments, an antigen binding fragment of the invention is generated by treating an altered antibody of the invention with an appropriate protease (e.g. pepsin, papain) under appropriate conditions. Exemplary antigen-binding fragments include Fv, Fab, Fab′, and (Fab′)₂. In preferred embodiments, the antigen-binding fragment of the invention is an altered antigen-binding fragment comprising an altered polypeptide having an altered constant region domain with at least one cysteine residue or analog thereof. In one exemplary embodiment, an altered antigen binding fragment of the invention comprises an altered CH1-containing polypeptide described supra. In another exemplary embodiment, an altered antigen binding fragment of the invention comprises an altered CL-containing polypeptide described supra.

In another embodiment, an altered antibody variant comprises an altered polypeptide comprising (i) a heavy chain variable region (VH) sequence derived, (ii) a light chain variable region (VL) sequence, and (iii) at least one altered constant region or portion thereof having an engineered cysteine residue and analog thereof. Said heavy chain and light chain variable sequences may be derived from any antibody, including any of the antibodies described supra.

In exemplary embodiments, the altered antibody variant comprises a single chain variable region sequence (ScFv). Single chain variable region sequences comprise a single polypeptide having one or more antigen binding sites, e.g., a V_{L} domain linked by a flexible linker to a V_{H} domain. ScFv molecules can be constructed in a V_{H}-linker-V_{L} orientation or V_{L}-linker-V_{H} orientation. The flexible hinge that links the V_{L} and V_{H} domains that make up the antigen binding site preferably comprises from about 10 to about 50 amino acid residues. An exemplary connecting peptide for this purpose is (Gly4Ser)3 (SEQ ID NO: 60) (Huston et al. (1988). PNAS, 85:5879). Other connecting peptides are known in the art. Antibodies having single chain variable region sequences (e.g. single chain antibodies) and methods of making said single chain antibodies are well-known in the art (see e.g., Ho et al. 1989. Gene 77:51; Bird et al. 1988 Science 242:423; Pantoliano et al. 1991. Biochemistry 30:10117; Milenic et al. 1991. Cancer Research 51:6363; Takkinen et al. 1991. Protein Engineering 4:837).

Altered single chain antibodies of the invention comprise altered polypeptides having at least one scFv and at least one altered constant region or portion thereof. In one embodiment, a single chain antibody of the invention may comprise at least one scFv and an altered CH1 constant region described supra. In another embodiment, a single chain antibody of the invention comprises a polypeptide having at least one scFv and an altered CH3 constant region described supra. In another embodiment, a single chain antibody of the invention comprises a polypeptide having at least one scFv and an altered CH1 constant region described supra.

In certain embodiments, an altered antibody variant of the invention is a tetravalent antibody which is produced by fusing a DNA sequence encoding an altered antibody with a ScFv molecule. For example, in one embodiment, these sequences are combined such that the ScFv molecule is linked at its N-terminus to an altered CH3 domain of an altered antibody via a flexible linker (e.g., a gly/ser linker such as (Gly4Ser)₃ (SEQ ID NO: 60).

In another embodiment a tetravalent antibody of the invention can be made by fusing an ScFv molecule to a connecting peptide, which is fused to an altered CH 1 domain to construct an ScFv-Fab tetravalent molecule. (Coloma and Morrison. 1997. Nature Biotechnology. 15:159; WO 95/09917).

In another embodiment, an altered antibody variant of the invention is an altered minibody. Altered minibodies of the invention are dimeric molecules made up of two polypeptide chains each comprising an ScFv molecule which is fused to a altered CH3 domain or portion thereof via a connecting peptide. Minibodies can be made by constructing an ScFv component and connecting peptide-CH3 component using methods described in the art (see, e.g., U.S. Pat. No. 5,837,821 or WO 94/09817A1). These components can be isolated from separate plasmids as restriction fragments and then ligated and recloned into an appropriate vector. Appropriate assembly can be verified by restriction digestion and DNA sequence analysis.

Methods of making single chain antibodies are well known in the art, e.g., Ho et al. (1989), Gene, 77:51; Bird et al. (1988), Science 242:423; Pantoliano et al. (1991), Biochemistry 30:10117; Milenic et al. (1991), Cancer Research, 51:6363; Takkinen et al. (1991), Protein Engineering 4:837. Minibodies of the invention can be made by constructing an ScFv component and a connecting peptide component using methods described in the art (see, e.g., U.S. Pat. No. 5,837,821 or WO 94/09817A1), and linking a ScFv-connecting peptide construct to an altered CH3 domain having at least one engineered cysteine residue or portion thereof. These components can be isolated from separate plasmids as restriction fragments and then ligated and recloned into an appropriate vector. Appropriate assembly can be verified by restriction digestion and DNA sequence analysis. In one embodiment, a minibody of the invention comprises a connecting peptide. In one embodiment, the connecting peptide comprises a gly/ser linker, e.g., GGGSSGGGSGG (SEQ ID NO: 61).

In another embodiment, an altered tetravalent minibody can be constructed. Altered tetravalent minibodies can be constructed in the same manner as minibodies, except that two ScFv molecules are linked using a flexible linker, e.g., having an amino acid sequence (G4S)₄G3AS (SEQ ID NO: 62). The linked scFv-scFv construct is then joined to an altered CH3 domain of the invention.

In another embodiment, an altered antibody of the invention comprises an altered diabody. Diabodies are dimeric, tetravalent molecules each having a polypeptide similar to scFv molecules, but usually having a short (less than 10 and preferably 1-5) amino acid residue linker connecting both variable domains, such that the V_{L} and V_{H} domains on the same polypeptide chain cannot interact. Instead, the V_{L} and V_{H} domain of one polypeptide chain interact with the V_{H} and V_{L} domain (respectively) on a second polypeptide chain (see, for example, WO 02/02781). In certain embodiments, the altered diabodies of the invention comprise an altered CH3 domain or portion thereof having at least one engineered cysteine or analog thereof. In other embodiments, the altered diabodies of the invention comprise an altered CH1 or CL domain of the invention.

C. Altered Fusion Proteins

The altered binding proteins of the invention include altered fusion proteins which comprise one or more altered constant region domains of the invention. The fusion proteins of the invention comprise a binding domain (which comprises at least one binding site) and at least one altered constant region or portion thereof. In preferred embodiments, a fusion protein comprises at least a complete Fc region having at least one altered heavy chain constant region domain (e.g. an altered CH2 or CH3 domain).

Ordinarily, the binding domain is fused C-terminally to the N-terminus of an altered constant region (e.g. an Fc region) and in place of the variable region. Any transmembrane regions or lipid or phospholipids anchor recognition sequences of ligand binding receptor are preferably inactivated or deleted prior to fusion. DNA encoding the ligand or ligand binding partner is cleaved by a restriction enzyme at or proximal to the 5′ and 3′ ends of the DNA encoding the desired ORF segment. The resultant DNA fragment is then readily inserted into DNA encoding a heavy chain constant region. The precise site at which the fusion is made may be selected empirically to optimize the secretion or binding characteristics of the soluble fusion protein. DNA encoding the fusion protein is then transfected into a host cell for expression.

In one embodiment, a fusion protein combines the binding domain(s) of the ligand or receptor (e.g. the extracellular domain (ECD) of a receptor) with at least one altered constant region or portion thereof. In one embodiment, when preparing the fusion proteins of the present invention, nucleic acid encoding the binding domain of the ligand or receptor domain will be fused C-terminally to nucleic acid encoding the N-terminus of an altered constant region or portion thereof. N-terminal fusions are also possible. In exemplary embodiments, fusions are made to the C-terminus of altered Fc region, or immediately N-terminal to the hinge domain of an altered Fc region.

In one embodiment, the altered constant region domain of the fusion protein includes substantially the entire Fc region of an immunoglobulin, beginning in the hinge region just upstream of the papain cleavage site which defines IgG Fc chemically (i.e. residue 216, taking the first residue of heavy chain constant region to be 114) and ending at its C-terminus. The precise site at which the fusion is made is not critical; particular sites are well known and may be selected in order to optimize the biological activity, secretion, or binding characteristics of the molecule. Methods for making fusion proteins are known in the art.

In exemplary embodiments, an altered fusion protein of the invention may comprise a ligand binding domains or receptor binding domains derived from one of the following proteins:

1. Cytokines and Cytokine Receptors

Cytokines have pleiotropic effects on the proliferation, differentiation, and functional activation of lymphocytes. Various cytokines, or receptor binding portions thereof, can be utilized in the fusion proteins of the invention. Exemplary cytokines include the interleukins (e.g. IL-1, IL-2, IL-3, IL-4, IL-5, IL-6, IL-7, IL-8, IL-10, IL-11, IL-12, IL-13, and IL-18), the colony stimulating factors (CSFs) (e.g. granulocyte CSF (G-CSF), granulocyte-macrophage CSF (GM-CSF), and monocyte macrophage CSF (M-CSF)), tumor necrosis factor (TNF) alpha and beta, and interferons such as interferon-α, β, or γ (U.S. Pat. Nos. 4,925,793 and 4,929,554).

Cytokine receptors typically consist of a ligand-specific alpha chain and a common beta chain. Exemplary cytokine receptors include those for GM-CSF, IL-3 (U.S. Pat. No. 5,639,605), IL-4 (U.S. Pat. No. 5,599,905), IL-5 (U.S. Pat. No. 5,453,491), IFNγ (EP0240975), and the TNF family of receptors (e.g., TNFα (e.g. TNFR-1 (EP 417, 563), TNFR-2 (EP 417,014) lymphotoxin beta receptor).

2. Adhesion Proteins

Adhesion molecules are membrane-bound proteins that allow cells to interact with one another. Various adhesion proteins, including leukocyte homing receptors and cellular adhesion molecules, of receptor binding portions thereof, can be incorporated in a fusion protein of the invention. Leucocyte homing receptors are expressed on leucocyte cell surfaces during inflammation and include the β-1 integrins (e.g. VLA-1, 2, 3, 4, 5, and 6) which mediate binding to extracellular matrix components, and the β2-integrins (e.g. LFA-1, LPAM-1, CR3, and CR4) which bind cellular adhesion molecules (CAMs) on vascular endothelium. Exemplary CAMs include ICAM-1, ICAM-2, VCAM-1, and MAdCAM-1. Other CAMs include those of the selectin family including E-selectin, L-selectin, and P-selectin.

3. Chemokines

Chemokines, chemotactic proteins which stimulate the migration of leucocytes towards a site of infection, can also be incorporated into a fusion protein of the invention. Exemplary chemokines include Macrophage inflammatory proteins (MIP-1-α and MIP-1-β), neutrophil chemotactic factor, and RANTES (regulated on activation normally T-cell expressed and secreted).

4. Growth Factors and Growth Factor Receptors

Growth factors or their receptors (or receptor binding or ligand binding portions thereof) may be incorporated in the fusion proteins of the invention. Exemplary growth factors include Vascular Endothelial Growth Factor (VEGF) and its isoforms (U.S. Pat. No. 5,194,596); Fibroblastic Growth Factors (FGF), including aFGF and bFGF; atrail natriuretic factor (ANF); hepatic growth factors (HGFs; U.S. Pat. Nos. 5,227,158 and 6,099,841), neurotrophic factors such as bone-derived neurotrophic factor (BDNF), neurotrophin-3, -4, -5, or -6 (NT-3, NT-4, NT-5, or NT-6), or a nerve growth factor such as NGF-β platelet-derived growth factor (PDGF) (U.S. Pat. Nos. 4,889,919, 4,845,075, 5,910,574, and 5,877,016); transforming growth factors (TGF) such as TGF-alpha and TGF-beta (WO 90/14359), osteoinductive factors including bone morphogenetic protein (BMP); insulin-like growth factors-I and -II (IGF-I and IGF-II; U.S. Pat. Nos. 6,403,764 and 6,506,874); Erythropoietin (EPO); stem-cell factor (SCF), thrombopoietin (c-Mpl ligand), and the Wnt polypeptides (U.S. Pat. No. 6,159,462).

Exemplary growth factor receptors which may be used as targeting receptor domains of the invention include EGF receptors; VEGF receptors (e.g. Flt1 or Flk1/KDR), PDGF receptors (WO 90/14425); HGF receptors (U.S. Pat. Nos. 5,648,273, and 5,686,292), and neurotrophic receptors including the low affinity receptor (LNGFR), also termed as p75^{NTR} or p75, which binds NGF, BDNF, and NT-3, and high affinity receptors that are members of the trk family of the receptor tyrosine kinases (e.g. trkA, trkB (EP 455,460), trkC (EP 522,530)).

5. Hormones

Exemplary growth hormones for use as targeting agents in the fusion proteins of the invention include renin, human growth hormone (HGH; U.S. Pat. No. 5,834,598), N-methionyl human growth hormone; bovine growth hormone; growth hormone releasing factor; parathyroid hormone (PTH); thyroid stimulating hormone (TSH); thyroxine; proinsulin and insulin (U.S. Pat. Nos. 5,157,021 and 6,576,608); follicle stimulating hormone (FSH), calcitonin, luteinizing hormone (LH), leptin, glucagons; bombesin; somatropin; mullerian-inhibiting substance; relaxin and prorelaxin; gonadotropin-associated peptide; prolactin; placental lactogen; OB protein; or mullerian-inhibiting substance.

6. Clotting Factors

Exemplary blood coagulation factors for use as targeting agents in the fusion proteins of the invention include the clotting factors (e.g., factors V, VII, VIII, X, IX, XI, XII and XIII, von Willebrand factor); tissue factor (U.S. Pat. Nos. 5,346,991, 5,349,991, 5,726,147, and 6,596,84); thrombin and prothrombin; fibrin and fibrinogen; plasmin and plasminogen; plasminogen activators, such as urokinase or human urine or tissue-type plasminogen activator (t-PA).

### IV. MULTISPECIFIC ALTERED BINDING PROTEINS

In one embodiment, an altered binding protein of the invention is multispecific, i.e., has at least one binding site that binds to a first molecule or epitope of a molecule and at least one second binding site that binds to a second molecule of epitope of a molecule.

In one embodiment, an altered binding protein of the invention is bispecific. Bispecific binding proteins can bind to two different target sites, e.g., on the same target molecule or on different target molecules. For example, in the case of the altered antibodies of the invention, a bispecific variant thereof can bind to two different epitopes, e.g., on the same antigen or on two different antigens. Bispecific binding proteins can be used, e.g., in diagnostic and therapeutic applications. For example, they can be used to immobilize enzymes for use in immunoassays. They can also be used in diagnosis and treatment of cancer, e.g., by binding both to a tumor associated molecule and a detectable marker (e.g., a chelator which tightly binds a radionuclide. Bispecific molecules can also be used for human therapy, e.g., by directing cytotoxicity to a specific target (for example by binding to a pathogen or tumor cell and to a cytotoxic trigger molecule, such as the T cell receptor or the Fcγ receptor. Bispecific antibodies can also be used, e.g., as fibrinolytic agents or vaccine adjuvants.

Examples of bispecific altered binding proteins include those with at least two arms directed against different tumor cell antigens; bispecific altered binding proteins with at least one arm directed against a tumor cell antigen and at least one arm directed against a cytotoxic trigger molecule (such as anti-Fc.gamma.RI/anti-CD15, anti-p185.sup.HER2/Fc.gamma.RIII (CD16), anti-CD3/anti-malignant B-cell (1D10), anti-CD3/anti-p185.sup.HER2, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3); bispecific altered binding proteins with at least one arm which binds specifically to a tumor antigen and at least one arm which binds to a toxin (such as anti-saporin/anti-Id-1, anti-CD22/anti-saporin, anti-CD7/anti-saporin, anti-CD38/anti-saporin, anti-CEA/anti-ricin A chain, anti-interferon-.alpha.(IFN-.alpha.)/anti-hybridoma idiotype, anti-CEA/anti-vinca alkaloid); bispecific altered binding proteins for converting enzyme activated prodrugs (such as anti-CD30/anti-alkaline phosphatase (which catalyzes conversion of mitomycin phosphate prodrug to mitomycin alcohol)); bispecific altered binding proteins which can be used as fibrinolytic agents (such as anti-fibrin/anti-tissue plasminogen activator (tPA), anti-fibrin/anti-urokinase-type plasminogen activator (uPA)); bispecific altered binding proteins for targeting immune complexes to cell surface receptors (such as anti-low density lipoprotein (LDL)/anti-Fc receptor (e.g. Fc.gamma.RI, Fc.gamma.RII or Fc.gamma.RIII)); bispecific altered binding proteins for use in therapy of infectious diseases (such as anti-CD3/anti-herpes simplex virus (HSV), anti-T-cell receptor:CD3 complex/anti-influenza, anti-Fc.gamma.R/anti-HIV; bispecific binding polypeptides for tumor detection in vitro or in vivo such as anti-CEA/anti-EOTUBE, anti-CEA/anti-DPTA, anti-p185HER2/anti-hapten); bispecific altered binding proteins as vaccine adjuvants (see Fanger et al., supra); and bispecific binding molecules as diagnostic tools (such as anti-rabbit IgG/anti-ferritin, anti-horse radish peroxidase (HRP)/anti-hormone, anti-somatostatin/anti-substance P, anti-HRP/anti-FITC, anti-CEA/anti-.beta.-galactosidase (see Nolan et al., supra)). Examples of trispecific antibodies include anti-CD3/anti-CD4/anti-CD37, anti-CD3/anti-CD5/anti-CD37 and anti-CD3/anti-CD8/anti-CD37.

In a preferred embodiment, a bispecific altered binding protein of the invention has one arm which binds to CRIPTO-I. In another preferred embodiment, a bispecific altered binding protein of the invention has one arm which binds to LTβR. In another preferred embodiment, a bispecific altered binding protein of the invention has one arm which binds to TRAIL-R2. In another preferred embodiment, a bispecific altered binding protein of the invention has one arm which binds to LTβR and one arm which binds to TRAIL-R2.

Multispecific altered binding proteins of the invention may be monovalent for each specificity or be multivalent for each specificity. For example, an altered antibody or altered fusion protein of the invention may comprise one binding site that reacts with a first target molecule and one binding site that reacts with a second target molecule or it may comprise two binding sites that react with a first target molecule and two binding sites that react with a second target molecule.

Methods of producing bispecific proteins are well known in the art. For example, synthetic or recombinant technology can be used to produce bispecific molecules, e.g., diabodies, single-chain diabodies, tandem scFvs, etc. Exemplary techniques for producing bispecific proteins are known in the art (e.g., Kontermann et al. Methods in Molecular Biology Vol. 248: Antibody Engineering: Methods and Protocols. Pp 227-242 US 2003/0207346 A1 and the references cited therein). In one embodiment, multispecific binding proteins of the invention are prepared using methods such as those described e.g., in US 2003/0207346 A1 or U.S. Pat. No. 5,821,333, or US2004/0058400.

As used herein the phrase “multispecific altered fusion protein” designates altered fusion proteins (as hereinabove defined) having at least two binding specificities (i.e. combining two or more binding domains of a ligand or receptor). Multispecific altered fusion proteins can be assembled as heterodimers, heterotrimers or heterotetramers, essentially as disclosed in WO 89/02922 (published Apr. 6, 1989), in EP 314, 317 (published May 3, 1989), and in U.S. Pat. No. 5,116,964 issued May 2, 1992. Preferred multispecific altered fusion proteins are bispecific. Examples of bispecific fusion proteins include CD4-IgG/TNFreceptor-IgG and CD4-IgG/L-selectin-IgG. The last mentioned molecule combines the lymph node binding function of the lymphocyte homing receptor (LHR, L-selectin), and the HIV binding function of CD4, and finds potential application in the prevention or treatment of HIV infection, related conditions, or as a diagnostic.

### V. CONJUGATION OF EFFECTOR MOIETIES TO ALTERED POLYPEPTIDES

In one aspect, the invention pertains to a method for modifying an altered polypeptide and thereby producing a modified polypeptide (e.g. a modified binding polypeptide). In certain embodiments, the method comprises providing an altered polypeptide (e.g. an altered polypeptide), providing an effector moiety, and combining the altered polypeptide and the effector moeity under suitable conditions to effect conjugation such that the altered polypeptide is modified.

In one embodiment, the modified binding proteins of the invention may be prepared by directly linking one or more effector moieties to an engineered cysteine residue or analog thereof of the altered binding polypeptide, wherein the effector moiety is suitably reactive with the engineered free cysteine or analog thereof. In another embodiment, the modified polypeptides of the invention may be prepared by indirectly conjugating one or more effector moieties to the free cysteine of the altered polypeptide via one or more linking moieties. Preferably, the linking agent is a bifunctional agent that contains both a thiol modification group for covalent linkage to the engineered free cysteine or analog thereof and at least one attachment moiety (e.g. a second thiol modification moiety) for covalent or non-covalent linkage to an effector moiety. Art recognized chemistries for covalently and specifically linking effector moieties (e.g. small molecules, nucleic acids, polymers, peptides, proteins, chemotherapeutics, and other types of molecules) to the thiol or sulfhydryl or other functional group in cysteine or cysteine analog side chains can be found, for example, in Hermanson, G. T., Bioconjugate Techniques, Academic Press (1996). Exemplary art recognized thiol modification groups include, but are not limited to, activated acyl groups (e.g., alpha-haloacetates, chloroacetic acid, or chloroacetamide), activated alkyl groups, Michael acceptors such as maleimide or acrylic groups, groups which react with sulfhydryl moieties via redox reactions, and activated di-sulfide groups. The thiol moieties may also be modified by reaction with bromotrifluoroacetone, alpha-bromo-beta-(5-imidazoyl)propionic acid, chloroacetyl phosphate, N-alkylmaleimides, 3-nitro-2-pyridyl disulfide, methyl-2-pyridyl disulfide, p-chloromercuribenzoate, 2-chloromercuri-4-nitrophenol, or chloro-7-nitrobenzo-2-oxa-1,3-diazole.

Linking agents containing thiol modification groups and preferably also contain an attachment group for covalent linkage to an effector moiety. Alternatively, linking agents may contain an attachment group for non-covalent linkage, e.g chelation, to the effector moiety. Exemplary chelating moieties include DOTA, PA-DOTA, TRITA, DTPA, IB4M-DTPA, CHX-A-DTPA, or TETA (see McCall M J et al., Bioconjugate Chem., 1: 222-6, 1990).

In another exemplary embodiment, highly specific thiol modification reagents can be used to modify altered binding proteins where an engineered cysteine residue or analog thereof is present at its C-terminus. The unique 1,2-amino thiol structure of the C-terminal engineered cysteine residue can be chemically or enzymatically modified with these agents, to form novel, non-thioether linkages with effector moieties and/or linking moieties. For example, relatively stable thiazolidine linkages with C-terminal engineered cysteine may be formed by glyoxylyl-based reagents (Zhao et al., Bioconjugate Chem., 10: 424-30, 1999).

Furthermore, the thiol of an engineered cysteine residue or analog thereof may be chemically derivatized to form a “cysteine derivative”. For example, an engineered cysteine may be derivatized into a ketone, an aldehyde, or other reactive carbonyl species. These species can then be reacted with hydrazines, hydrazides, or O-hydroxyl amines to make hydrazones, semicarbazones, or oxime linking moieties. The effector moieties may be directed attached to the linking moiety or indirectly attached to it through a bridging moiety.

The linking moiety may optionally further comprise a bridging moiety that functions to bridge the effector moiety and the altered polypeptide and provide a chemical spacer therebetween. Where the bridging moiety is a peptide, an affinity tag sequence (e.g. a His(6) tag, SEQ ID NO: 59) may optionally be included within the bridging moiety to facilitate downstream purification of the modified binding protein.

In one aspect of the invention, the altered polypeptide (e.g. altered binding polypeptide), effector moiety, and/or linking moiety are conjugated in vitro. In one embodiment, wherein the effector moiety is a protein, the effector moiety and altered polypeptide are expressed by separate expression plasmid constructs, and following purification, are conjugated in vitro with the chemical linker. In another embodiment, the altered polypeptide is expressed from an expression plasmid and the effector peptide is produced by solid-phase peptide synthesis, and following purification, are chemically linked in vitro.

In another aspect, where the effector moiety is a non-genetically encoded diagnostic or therapeutic agent, the effector moiety may be produced by synthetic methods or purified from a natural source. In one embodiment, the effector moiety is conjugated, e.g., through the linking moeity, to the altered polypeptide produced by recombinant DNA methods or solid-phase protein synthesis.

Conjugation reactions can be conducted by combining an altered polypeptide with effector moiety and/or chemical linking agent in an appropriate reaction buffer and incubating the mixture for a suitable time and under suitable conditions to affect the desired degree of conjugation. Suitable conditions include the proper salt concentration, pH, temperature, or the presence of an exogenous molecule. The reaction conditions and incubation time depend in part on the nature of the reactants used and can be determined by one skilled in the art by standard methods, including but not limited to monitoring the progress of the reaction by analyzing the reaction mixture by size exclusion HPLC or SDS-PAGE. The number of free cysteines in a particular altered polypeptide preparation, or the number of sites available for modification, can be determined before conjugation using methods that are well known in the art. For example, the number of free thiols per altered polypeptide can be determined in an analytical sample of known protein concentration by titrating the sample with 4,4′-dithiodipyridine, Ellman's reagent, DTNB, or other thiol-reactive agents (Talgoy et al., Can. J. Biochem., 57: 822-9, 1979). Where the number of free thiols exceed those desired, or are insufficient, the number of free thiols may be adjusted by use of agents which block excess free cysteines (e.g. glutathione) or reduce bonded cysteines (e.g MEA).

The efficiency and stoichiometry (if more than one free cysteine) of the conjugation reaction can be assessed by analytical scale thin-layer chromatography of a altered polypeptide sample which is conjugated to a radiolabelled effector moiety (Stimmel et al, J. Biol. Chem., 275(39): 30445-50, 2000).

The concentration of the reactants can be adjusted for higher modification efficiency, desired stoichiometry (if more than one free cysteine), and optimum reaction yield. Normally, higher reactant concentrations are advantageous for increased yield. Any methods known in the art can be used to determine the concentrations of protein, such as but is not limited to the Bradford assay or the bichinchoninic acid method using BSA as a standard (Smith et al., (1985), Anal. Biochem. 150: 76-85).

It shall be readily apparent to those skilled in the art that the methods of the invention can be used to modify virtually any altered polypeptide of interest, including any polypeptide fragment or protein domain thereof, with an effector moiety (E) in order to introduce a novel biological functionality to the altered polypeptide.

### VI) MODIFIED BINDING POLYPEPTIDES

In certain embodiments, the invention pertains, at least in part, to a modified binding polypeptide of the formula:

Pro-(X—[Y-E_{q}]ₘ)ₙ (I)

wherein

Pro is an altered binding polypeptide of the invention described supra;

X is an engineered cysteine residue, an analog thereof, or a cysteine derivative thereof,

Y is a linking moiety or a covalent bond, independently selected for each occurrence;

E is an independently selected effector moiety for each occurrence; and

q, m and n are each independently selected positive integers for each occurrence.

In a further embodiment, m, n, and q are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

A. Linking Moieties (Y)

Linking moieties may be employed to conjugate the altered binding polypeptide (Pro-X), with an effector moiety (E) of the invention. In some embodiments, the linking moiety contains at least one thiol modification group capable of forming a thioether linkage (S) with an engineered free cysteine in the altered binding polypeptide and a second covalent or noncovalent linkage with the effector moiety (E). Where the effector moiety is a peptide, the linker moiety may form a second thioether bond with a native free cysteine in the effector protein or peptide. In certain alternative embodiments, the linking moiety (Y) is linked to the polypeptide by cleaving one or more disulfide bonds formed by the engineered cysteine.

In a further embodiment, the linking moiety may comprise a spacer moiety. The linking moieties of the invention may be cleavable or non-cleavable. In one embodiment, the cleavable linking moiety is a redox-cleavable linking moiety, such that the linking moiety is cleavable in environments with a lower redox potential, such the cytoplasm and other regions with higher concentrations of molecules with free sulfhydryl groups. Examples of linking moieties that may be cleaved due to a change in redox potential include those containing disulfides. The cleaving stimulus can be provided upon intracellular uptake of the modified binding polypeptide where the lower redox potential of the cytoplasm facilitates cleavage of the linking moiety. In another embodiment, a decrease in pH triggers the release of the effector moiety cargo into the target cell. The decrease in pH is implicated in many physiological and pathological processes, such as endosome trafficking, tumor growth, inflammation, and myocardial ischemia. The pH drops from a physiological 7.4 to 5-6 in endosomes or 4-5 in lysosomes. Examples of acid sensitive linking moieties which may be used to target lysosomes or endosomes of cancer cells, include those with acid-cleavable bonds such as those found in acetals, ketals, orthoesters, hydrazones, trityls, cis-aconityls, or thiocarbamoyls (see for example, Willner et al., (1993), Bioconj. Chem., 4: 521-7, U.S. Pat. Nos. 4,569,789, 4,631,190, 5,306,809, and 5,665,358). Other exemplary acid-sensitive linking moieties comprise dipeptide sequences Phe-Lys and Val-Lys (King et al., (2002), J. Med. Chem., 45: 4336-43). The cleaving stimulus can be provided upon intracellular uptake trafficking to low pH endosomal compartments (e.g. lysosomes). Other exemplary acid-cleavable linking moieties are the branched cleavable linking moieties that contain two or more acid cleavable bonds for attachment of two or more effector moieties (King et al., (1999), Bioconj. Chem., 10: 279-88; WO 98/19705).

Cleavable linking moieties may be sensitive to biologically supplied cleaving agents that are associated with a particular target cell, for example, lysosomal or tumor-associated enzymes. Examples of linking moieties that can be cleaved enzymatically include, but are not limited to, peptides and esters. Exemplary enzyme cleavable linking moieties include those that are sensitive to tumor-associated proteases such as Cathepsin B or plasmin (Dubowchik et al., (1999), Pharm. Ther., 83: 67-123; Dubowchik et al., (1998), Bioorg. Med. Chem. Lett., 8: 3341-52; de Groot et al., (2000), J. Med. Chem., 43: 3093-102; de Groot et al., (1999) 42: 5277-83). Cathepsin B-cleavable sites include the dipeptide sequences valine-citrulline and phenylalanine-lysine (Doronina et al., (2003), Nat. Biotech., 21(7): 778-84); Dubowchik et al., (2002), Bioconjug. Chem., 13: 855-69). Other exemplary enzyme-cleavable sites include those formed by oligopeptide sequences of 4 to 16 amino acids (e.g., Suc-β-Ala-Leu-Ala-Leu) which recognized by trouse proteases such as Thimet Oliogopeptidase (TOP), an enzyme that is preferentially released by neutrophils, macrophages, and other granulocytes (U.S. patent application Ser. No. 09/789,442, filed Jun. 11, 2001).

Alternatively, the cleavable linking moieties may not be cleavable at the site of the target cells, but cleavable at the site of a non-target cell. In other words, the linking moiety is more stable upon localization to the target cell. Stable linking moieties are preferably introduced into target binding polypeptides to form modified binding polypeptides that used for diagnostic purposes. Selectively stable bridging moieties therefore, provide a means to reduce non-specific localization of diagnostic antibodies at non-target sites. Preferred stable linking moieties include “metabolizable” sites comprising thiourea groups, peptides, esters, or disulfides that are selectively metabolized by non-target cells (Haseman et al., (1986), J. Nucl. Med., 12: 455-60). Non-specific localization of diagnostic modified binding polypeptide may be reduced by administering an exogenous enzyme or chemical cleaving agent which selectively cleaves a cleavable linking moiety at one or more non-target cells within the host. For example, the cleaving agent may be a compound that alters the pH or redox state at the non-target site (e.g. reducing kidney toxicity by acidifying the urine) or a compound that increases the reducing state of the non-target site. Exemplary urine acidifying agents include ammonium chloride (U.S. Pat. No. 5,171,563). In another embodiment, the linking moiety may be susceptible to cleavage by serum enzymes (e.g. serum esterases). Examples, of linking moieties susceptible to plasma hydrolysis include certain derivatives of glycolamides (Nielsen et al., (1987), J. Med. Chem., 30(3): 451-4; U.S. Pat. No. 5,171,563).

In another embodiment, the cleavable linking moiety may comprise a tripartite releasable Polyethylene Glycol (rPEG) cleavage site for controlled hydrolytic release of an effector moiety, such as a cytotoxin (Greenwald et al., (2003), Bioconjug. Chem., 14: 395-403). rPEG cleavage sites may contain a variable number of PEG substituents attached by cleavable groups (e.g., those containing ester, carbamate, or carbonate bonds) and non-cleavable groups (e.g. a p-substituted benzyl alcohols) to free amino groups of a peptide sequence. The hydrolysis rate of the rPEG cleavage site can be modified by the number of PEG groups and the choice of substituents on the cleavable and non-cleavable groups. Such linking moieties can be attached to one or more effector moieties with appropriate chemically reactive functional groups (e.g. a free amine or hydroxyl). In one embodiment, the linking moiety can be directly attached to one or more appropriately functionalized effector moieties via a covalent bond, such that the effector moiety may be released unaltered or attached to vestiges of the cleavable group. In another embodiment, the effector moiety may first be derivatized in preparation for direct reaction with the reactive functional group on the cleavable linking moiety.

B. Effector Moieties (E)

The effector moieties (E) of the invention, preferably, add a non-natural function to an altered binding polypeptide, without significantly altering the intrinsic activity of the altered binding polypeptide. The effector moiety may be, for example but not limited to, a therapeutic or diagnostic agent. A modified binding polypeptide of the invention may comprise one or more effector moieties, which may by the same of different.

i. Diagnostic Effector Moieties

An altered binding polypeptide may be modified using the methods of the invention to provide a modified binding polypeptide with improved diagnostic utility, e.g. to identify the location of a particular antigen or target cell.

Altered binding polypeptides may be modified with effector moieties (E) comprising diagnostic moieties, e.g. detectable labels such as biotin, fluorophores, chromophores, spin resonance probes, or radiolabels. Exemplary fluorophores include fluorescent dyes (e.g. fluorescein, rhodamine, and the like) and other luminescent molecules (e.g. luminal). A fluorophore may be environmentally-sensitive such that its fluorescence changes if it is located close to one or more residues in the modified binding polypeptide that undergo structural changes upon binding a substrate (e.g. dansyl probes). Exemplary radiolabels include small molecules containing atoms with one or more low sensitivity nuclei (¹³C, ¹⁵N, ²H, ¹²⁵I, ¹²⁴I, ¹²³I, ⁹⁹Tc, ⁴³K, ⁵²Fe, ⁶⁴Cu, ⁶⁸Ga, ¹¹¹In and the like). Preferably, the radionuclide is a gamma, photon, or positron-emitting radionuclide with a half-life suitable to permit activity or detection after the elapsed time between administration and localization to the imaging site.

Alternatively, altered binding polypeptides may be modified with effector moieties comprising diagnostic proteins. Exemplary diagnostic proteins include enzymes with fluorogenic or chromogenic activity, e.g. the ability to cleave a substrate which forms a fluorophore or chromophore as a product (i.e. reporter proteins such as luciferase). Other diagnostic proteins may have intrinsic fluorogenic or chromogenic activity (e.g., green, red, and yellow fluorescent bioluminescent aequorin proteins from bioluminescent marine organisms) or they may comprise a protein containing one or more low-energy radioactive nuclei (¹³C, ¹⁵N, ²H, ¹²⁵I, ¹²³I, ⁹⁹Tc, ⁴³K, ⁵²Fe, ⁶⁷Ga, ⁶⁸Ga, ¹¹¹In and the like).

With respect to the use of radiolabeled conjugates in conjunction with the present invention, polypeptides of the invention may be directly labeled (such as through iodination) or may be labeled indirectly through the use of a chelating agent. As used herein, the phrases “indirect labeling” and “indirect labeling approach” both mean that a chelating agent is covalently attached to a binding polypeptide and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“MX-DTPA”,) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, (1985), J. Nuc. Med. 26: 3328 and Carraguillo et al., (1985), J. Nuc. Med. 26: 67.

As used herein, the phrases “direct labeling” and “direct labeling approach” both mean that a radionuclide is covalently attached directly to the altered binging protein via the engineered cysteine residue. A particularly preferred radionuclide for direct labeling is ¹³¹I.

Those skilled in the art will appreciate that non-radioactive conjugates may also be assembled depending on the selected agent to be conjugated. For example, conjugates with biotin can be prepared e.g. by reacting the polypeptides with biotin analog containing a thiol modification reagent.

ii) Therapeutic Effector Moieties

An altered binding polypeptide may be modified using the methods of the invention to provide a modified binding polypeptide with improved therapeutic utility.

Altered binding polypeptides may be modified with effector moieties comprising therapeutic agents, e.g. high energy radionuclides or radiolabelled compounds, lipids, pharmaceutical agents, drug moieties, or prodrugs thereof. Radionuclides or radiolabels with high-energy ionizing radiation are capable of causing multiple strand breaks in nuclear DNA, leading to cell death. Exemplary high-energy radionuclides include: ⁹⁰Y, ¹²⁵I, ¹³¹I, ¹²³I ¹¹¹I, ¹⁰⁵R, ¹⁵³Sm, ⁶⁷Cu, ⁶⁷Ga, ¹⁶⁶Ho, ¹⁷⁷Lu, ¹⁸⁶Re and ¹⁸⁸Re. These isotopes typically produce high energy α- or β-particles which have a short path length. Such radionuclides kill cells to which they are in close proximity, for example neoplastic cells to which the conjugate has attached or has entered. They have little or no effect on non-localized cells and are essentially non-immunogenic. Alternatively, high-energy isotopes may be generated by thermal irradiation of an otherwise stable isotope, for example as in boron neutron-capture therapy (Guan et al., PNAS, 95: 13206-10, 1998).

In another embodiment, the effector moeity used in the present invention comprises a drug moiety. Exemplary drug moieties include anti-inflammatory, anticancer, anti-infective (e.g., anti-fungal, antibacterial, anti-parasitic, anti-viral, etc.), and anesthetic therapeutic agents. In a further embodiment, the drug moiety is an anti-cancer agent. Exemplary anti-cancer agents include, but are not limited to, cytostatics, enzyme inhibitors, gene regulators, cytotoxic nucleosides, tubulin binding agents or tubulin inhibitors, proteasome inhibitors, hormones and hormone antagonists, anti-angiogenesis agents, and the like.

Exemplary cytostatic anti-cancer agents include alkylating agents such as the anthracycline family of drugs (e.g. adriamycin, caminomycin, cyclosporin-A, chloroquine, methopterin, mithramycin, porfiromycin, streptonigrin, porfiromycin, anthracenediones, and aziridines). Other cytostatic anti-cancer agents include DNA synthesis inhibitors (e.g., methotrexate and dichloromethotrexate, 3-amino-1,2,4-benzotriazine 1,4-dioxide, aminopterin, cytosine β-D-arabinofuranoside, 5-fluoro-5′-deoxyuridine, 5-fluorouracil, ganciclovir, hydroxyurea, actinomycin-D, and mitomycin C), DNA-intercalators or cross-linkers (e.g., bleomycin, carboplatin, carmustine, chlorambucil, cyclophosphamide, cis-diammineplatinum(II) dichloride (cisplatin), melphalan, mitoxantrone, and oxaliplatin), and DNA-RNA transcription regulators (e.g., actinomycin D, daunorubicin, doxorubicin, homoharringtonine, and idarubicin). Other exemplary cytostatic agents that are compatible with the present invention include ansamycin benzoquinones, quinonoid derivatives (e.g. quinolones, genistein, bactacyclin), busulfan, ifosfamide, mechlorethamine, triaziquone, diaziquone, carbazilquinone, indoloquinone EO9, diaziridinyl-benzoquinone methyl DZQ, triethylenephosphoramide, and nitrosourea compounds (e.g. carmustine, lomustine, semustine).

Exemplary cytotoxic nucleoside anti-cancer agents include, for example, adenosine arabinoside, cytarabine, cytosine arabinoside, 5-fluorouracil, fludarabine, floxuridine, ftorafur, and 6-mercaptopurine.

Exemplary anti-cancer tubulin binding agents include taxoids (e.g. paclitaxel, docetaxel, taxane), nocodazole, rhizoxin, dolastatins (e.g. Dolastatin-10, -11, or -15), colchicine and colchicinoids (e.g. ZD6126), combretastatins (e.g. Combretastatin A-4, AVE-6032), and vinca alkaloids (e.g. vinblastine, vincristine, vindesine, and vinorelbine (navelbine)).

Exemplary anti-cancer hormones and hormone antagonists, include corticosteroids (e.g. prednisone), progestins (e.g. hydroxyprogesterone or medroprogesterone), estrogens, (e.g. diethylstilbestrol), antiestrogens (e.g. tamoxifen), androgens (e.g. testosterone), aromatase inhibitors (e.g. aminogluthetimide), 17-(allylamino)-17-demethoxygeldanamycin, 4-amino-1,8-naphthalimide, apigenin, brefeldin A, cimetidine, dichloromethylene-diphosphonic acid, leuprolide (leuprorelin), luteinizing hormone-releasing hormone, pifithrin-α, rapamycin, sex hormone-binding globulin, and thapsigargin.

Exemplary anti-cancer, anti-angiogenesis compounds included Angiostatin K1-3, DL-α-difluoromethyl-ornithine, endostatin, fumagillin, genistein, minocycline, staurosporine, and (±)-thalidomide.

Exemplary anti-cancer enzyme inhibitors include but are not limited to, S(+)-camptothecin, curcumin, (−)-deguelin, 5,6-dichlorobenz-imidazole 1-β-D-ribofuranoside, etoposide, formestane, fostriecin, hispidin, 2-imino-1-imidazolidineacetic acid (cyclocreatine), mevinolin, trichostatin A, tyrphostin AG 34, and tyrphostin AG 879.

Examplary anti-cancer gene regulators include 5-aza-2′-deoxycytidine, 5-azacytidine, cholecalciferol (vitamin D₃), 4-hydroxytamoxifen, melatonin, mifepristone, raloxifene, trans-retinal (vitamin A aldehydes), retinoic acid, vitamin A acid, 9-cis-retinoic acid, 13-cis-retinoic acid, retinol (vitamin A), tamoxifen, and troglitazone.

Other preferred classes of anti-cancer agents include, for example, the pteridine family of drugs, diynenes, and the podophyllotoxins. Particularly useful members of those classes include, for example, methopterin, podophyllotoxin, or podophyllotoxin derivatives such as etoposide or etoposide phosphate, leurosidine, vindesine, leurosine and the like.

Still other anti-cancer agents that are compatible with the teachings herein include auristatins (e.g. auristatin E and monomethylauristan E), geldanamycin, calicheamicin, gramicidin D, maytansanoids (e.g. maytansine), neocarzinostatin, topotecan, taxanes, cytochalasin B, ethidium bromide, emetine, tenoposide, colchicin, dihydroxy anthracindione, mitoxantrone, procaine, tetracaine, lidocaine, propranolol, puromycin, and analogs or homologs thereof.

Examples of certain drug moieties of the invention are shown below in Table 1:

TABLE 1

| Structure | Name | Possible Method of Action |
|---|---|---|
| 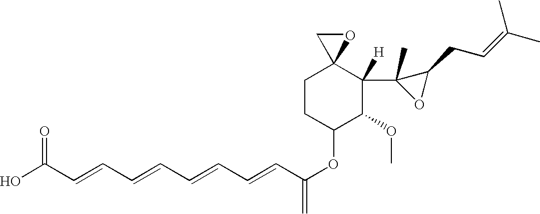 | Fumagillin | Inhibitor of endothelial cell proliferation and angiogenesis. |
| 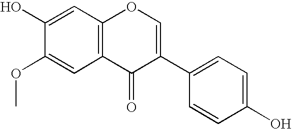 | Genistein | Antiangiogenic agent, down- regulates the transcription of genes involved in controlling angiogenesis. |
| 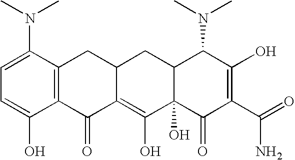 | Minocycline | Inhibits endothelial cell proliferation and angiogenesis. |
| 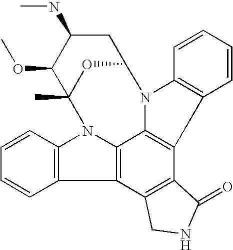 | Staurosporine | Blocks angiogenesis by inhibiting the upregulation of VEGF expression in tumor cells. |
| 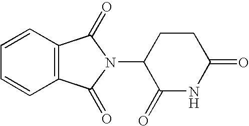 | (±)-Thalidomide | Selectively inhibits biosynthesis of tumor necrosis factor α (TNF-α); inhibits angiogenesis. |
| 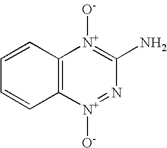 | 3-Amino-1,2,4- benzotriazine 1,4- dioxide | Hypoxia-activated antineoplastic agent. |
| 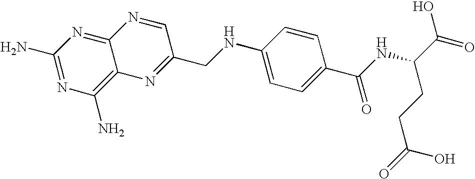 | Aminopterin | Folic acid antagonist; blocks thymidine biosynthesis by inhibiting dihydrofolate reductase. More potent, but more toxic, than methotrexate. |
| 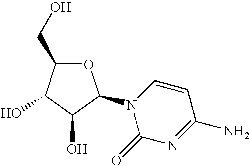 | Cytosine β-D- arabinofuranoside | Selective inhibitor of DNA synthesis. |
| 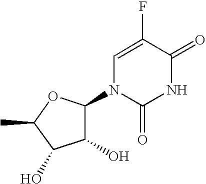 | 5-Fluoro-5′- deoxyuridine | Inhibits proliferation of tumors, cell lines or fibroblasts transformed by H-Ras or Trk oncogenes. |
| 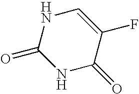 | 5-Fluorouracil | Inhibits thymidylate synthetase and depletes dTTP; it forms nucleotides that can be incorporated into RNA and DNA and induces p53- dependent apoptosis. |
| 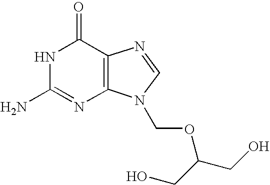 | Ganciclovir | In suicide gene therapy of solid tumors, the gene for Herpes simplex virus thymidine kinase is delivered to tumor cells and expressed, which in turn activates ganciclovir cytotoxicity. |
| 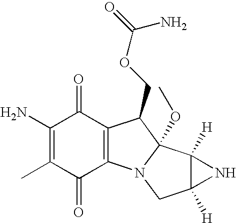 | Mitomycin C | Inhibits DNA synthesis, nuclear division, and proliferation of cancer cells. |
| 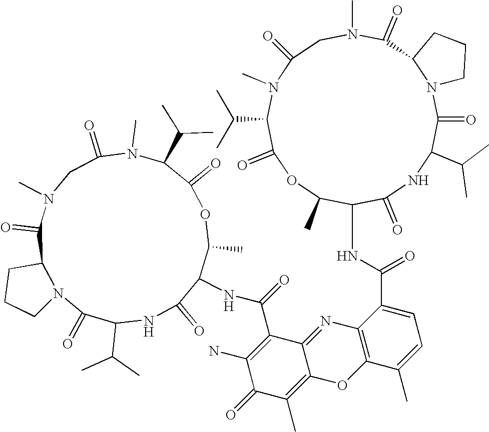 | Actinomycin D | Inhibits cell proliferation by complexing to DNA and blocking the production of mRNA by RNA polymerase; induces apoptosis. |
| 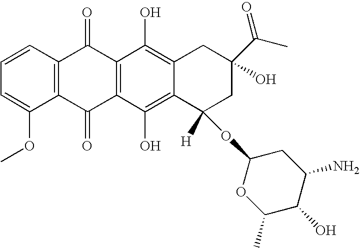 | Daunorubicin | Complexes to DNA and blocks production of mRNA by RNA polymerase. |
| 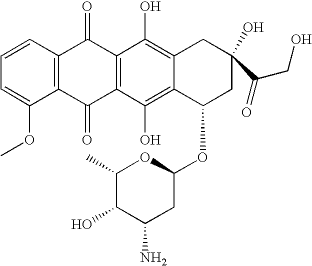 | Doxorubicin | Binds to DNA and inhibits reverse transcriptase and RNA polymerase. |
| 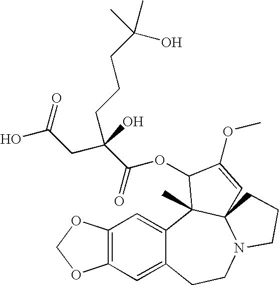 | Homoharringtonine | Binds to the 80S ribosome in eukaryotic cells and inhibits protein synthesis by interfering with chain elongation. |
| 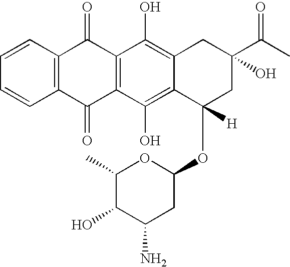 | Idarubicin | Anti-leukemia agent with higher DNA binding capacity and greater cytotoxicity than daunorubicin. |
| 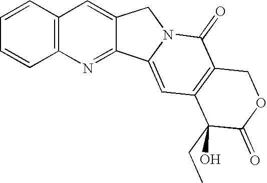 | S(+)-Camptothecin | Binds irreversibly to the DNA- topoisomerase I complex leading to the irreversible cleavage of DNA and the destruction of cellular topoisomerase I by the ubiquitin- proteasome pathway. Induces apoptosis in many normal and tumor cell lines. |
| 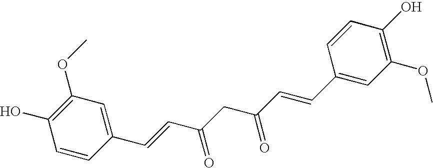 | Curcumin | Potent inhibitor of protein kinase C, EGFR tyrosine kinase and IκB kinase. Induces apoptosis in cancer cells. |
| 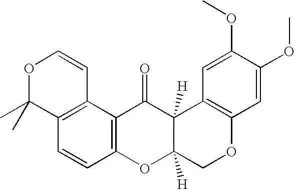 | (−)-Deguelin | Inhibitor of activated Akt. Does not affect MAPK, ERK 1/2 or JNK. |
| 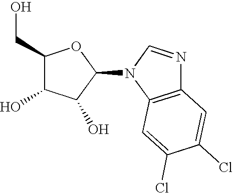 | 5,6- Dichlorobenzimidazole 1-β-D-ribofuranoside | Inhibitor of RNA synthesis, causes premature termination of transcription. |
| 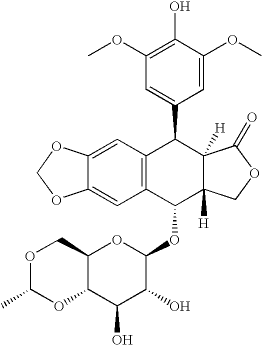 | Etoposide | Binds to the DNA-topoisomerase II complex to enhance cleavage and inhibit religation; inhibits synthesis of the oncoprotein Mdm2 and induces apoptosis in tumor lines that overexpress Mdm2. |
| 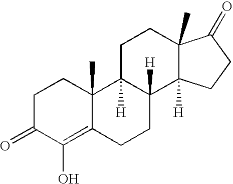 | Formestane | Aromatase inhibitor |
| 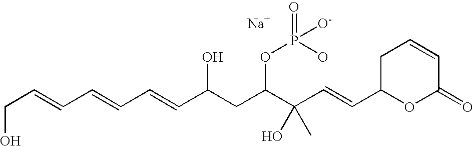 | Fostriecin | Interferes with the reversible phosphorylation of proteins that are critical for progression through the cell cycle. |
| 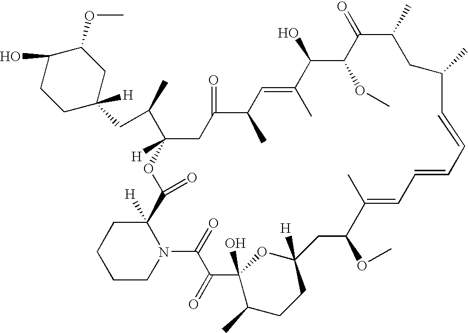 | Rapamycin | Inhibition of the molecular target of rapamycin (mTOR) mediates the antiproliferative and anticancer activity of rapamycin by blocking the PI3K/Akt pathway. |
| 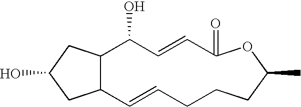 | Brefeldin A | Disrupts the structure and function of the Golgi apparatus. An activator of the sphingomyelin cycle. |
| 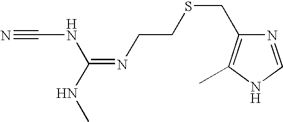 | Cimetidine | H₂ histamine receptor antagonist; I₁ imidazoline receptor agonist; anti-ulcer agent. Blocks cancer metastasis by inhibiting the expression of E-selectin on the surface of endothelial cells, thus blocking tumor cell adhesion. |
| 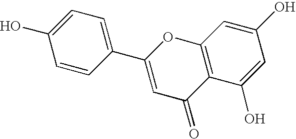 | Apigenin | Inhibits cell proliferation by arresting the cell cycle at the G2/M phase. Inhibition of growth through cell cycle arrest and induction of apoptosis appear to be related to induction of p53. Inhibitory effects on tumor promotion may also be due to inhibition of kinase activity and the resulting suppression of oncogene expression. It has also been reported to inhibit topoisomerase I catalyzed DNA religation and enhance gap junctional intercellular communication. |
| 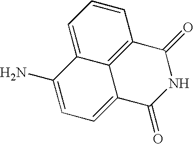 | 4-Amino-1,8- naphthalimide | Sensitizes cells to radiation- induced cell damage and enhances the cytotoxicity of 1-methyl-3- nitro-1-nitrosoguanidine. |
| 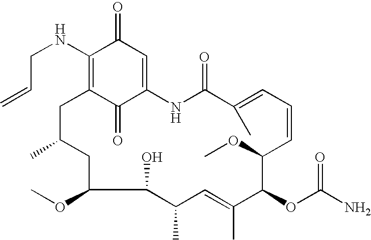 | 17-(Allylamino)-17- demethoxygeldana mycin | Inhibits the activity of oncogenic proteins such as N-ras, Ki-ras, c-Akt, and p185^{erB2}. Induces apoptosis. |
| 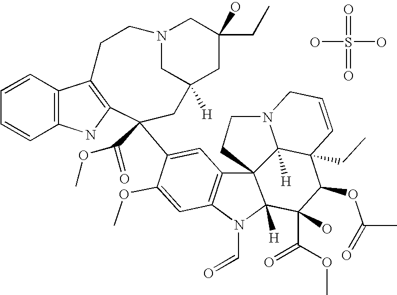 | Vincristine | Antimitotic agent. Inhibits microtubule assembly by binding tubulin and inducing self- association; depolymerizes pre- existing microtubules. Induces apoptosis in several tumor cell lines. |

Another class of compatible anti-cancer agents that may be used as drug moieties are radiosensitizing drugs that may be effectively directed to tumor or immunoreactive cells. Such drug moeities enhance the sensitivity to ionizing radiation, thereby increasing the efficacy of radiotherapy. Not to be limited by theory, but an antibody modified with a radiosensitizing drug moiety and internalized by the tumor cell would deliver the radiosensitizer nearer the nucleus where radiosensitization would be maximal. Antibodies which lose the radiosensitizer moiety would be cleared quickly from the blood, localizing the remaining radiosensitization agent in the target tumor and providing minimal uptake in normal tissues. After clearance from the blood, adjunct radiotherapy could be administered by external beam radiation directed specifically to the tumor, radioactivity directly implanted in the tumor, or systemic radioimmunotherapy with the same modified antibody.

In one preferred embodiment, a drug moiety of the invention is a maytansanoid drug moiety (Liu et al., (1996), PNAS, 93: 8618-23). Examples of maytansinoid drug moieties include moieties of formula (II):

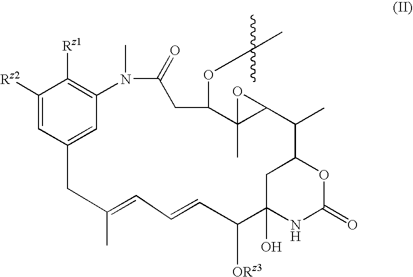

wherein

R^{Z1} is halogen or hydrogen; and

R^{Z2} and R^{Z3} are each hydrogen or lower alkyl.

In a further embodiment, R^{Z1} is chlorine and R^{Z2} and R^{Z3} are each methyl.

In another preferred embodiment, a drug moiety of the invention is a taxane drug moiety. Examples of taxane drug moieties include of compounds having the following formula (III):

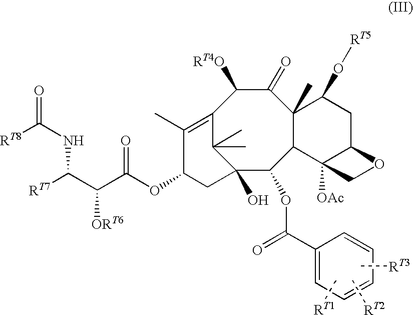

wherein:

R^{T1}, R^{T2}, and R^{T3} are each independently hydrogen, an electron withdrawing group, or an electron donating group;

R^{T4}, R^{T5}, R^{T6} are each independently a covalent bond to L, hydrogen, heterocyclic, an ester, an ether, a carbamate of the formula —CONR^{T10}R^{T11}, wherein R^{T10} and R^{T11} are each independently hydrogen, alkyl, alkenyl, alkynyl, acyl or aryl, provided that one of R^{T4}, R^{T5}, and R^{T6} is a covalent bond to L;

R^{T7} is alkyl, alkenyl, alkynyl, acyl or aryl; and

R^{T8} is alkoxy or aryl.

In another embodiment, R^{T1} is halogen (e.g., fluorine, chlorine, bromine, etc.), NO₂, CN, CHF₂, CF₃, —OCH₃, —OCH₂CH₃, or NR^{T11}R^{T12}, where R^{T11} and R^{T12} are each independently hydrogen, alkyl (e.g., C₁-C₁₀ alkyl), alkenyl, alkynyl, or aryl.

In another embodiment, R^{T4} is —COC₂H₅, —CH₂CH₃, —CONHCH₂CH₃, —CO-morpholino, —CO-piperidino, —CO-piperazino, or —CO—N-methylpiperizino.

In another embodiment, R^{T1}, R^{T2}, and R^{T3} are H or methoxy. In a further embodiment, R^{T1} is in the meta position when R^{T2} and R^{T3} are hydrogen or methoxy.

In another preferred embodiment, a drug moiety of the invention is a doxorubicin drug moiety having the following formula (IV):

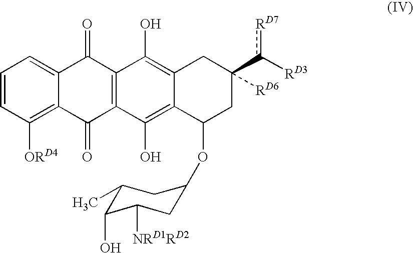

wherein:

Y is O or NR^{D5}, wherein R^{D5} is alkyl or hydrogen;

R^{D1} and R^{D2} are each hydrogen, or taken together a moiety of the formula (IIIa):

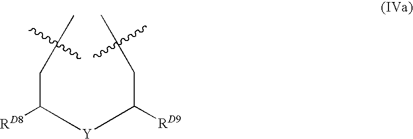

wherein

R^{D3} is alkyl;

R^{D4} is alkyl or hydrogen;

R^{D6} is hydroxy or alkyl;

R^{D7} is O or a covalent bond to L;

R^{D8} and R^{D9} are each a covalent bond to L, hydrogen, alkoxy, or alkyl; provided that one of R^{D1}, R^{D2}, and R^{D7} is a covalent bond to L.

In a further embodiment, R^{D5} is methyl. In another further embodiment, R^{D9} is alkoxy.

As noted previously, one skilled in the art may make chemical modifications to the desired drug moiety or protein in order to make reactions of that moeity more convenient for purposes of preparing modified binding polypeptides of the invention.

As an alternative or in addition to the therapeutic agents and drug moieties described above, the altered binding polypeptides of the invention may be modified with therapeutic protein or peptide effector moeities, e.g. binding polypeptides, peptide toxins, biological response modifiers, enzymes, or fragments thereof.

In one embodiment, the altered binding polypeptides of the invention may be modified with peptide toxins that are cytotoxic to cells including ricin, gelonin, pseudomonas exotoxin, diphtheria toxin, ricin subunit A, abrin, botulinum, cyanginosins, saxitoxin, shigatoxin, tetanus, tetrodotoxin, trichothecene, verrucologen or a toxic enzyme. In another embodiment, the effector moeities used in the present invention comprise biological response modifiers including the hormones, cytokines, chemokines, growth factors, cytokines, chemokines, and clotting factors cited above.

In certain embodiments, the altered binding polypeptides of the invention may be modified by conjugation to one or more other altered binding polypeptides of the invention. The resultant modified binding proteins have the following formula V:

Pro₁-(X₁—[Y₁—(X₂-Pro₂)_{q}]ₘ)ₙ (V)

wherein

Pro₁ and Pro₂ are independently selected from any of the altered binding polypeptides of the invention described supra;

X₁ and X₂ are independently selected from an engineered cysteine residue, an analog thereof, or a derivative thereof;

Y₁ and Y₂ are independently selected from a linking moiety or a covalent bond;

and

q, m and n are each independently selected positive integers for each occurrence.

In a further embodiment, m, n, and q are each independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30.

In one embodiment, Pro₁ and Pro₂ are the same. In another embodiment, Pro₁ and Pro₂ are different. In another embodiment, Pro₂ is optionally linked to at least one effector moiety via an engineered cysteine or analog thereof.

In another embodiment, n is 1 or 2 and Pro₁ is an altered CH3-containing binding polypeptide of the invention as described supra. In another embodiment, n is 2 and Pro₁ is an altered CH3-containing protein of the invention comprising two or more altered polypeptides, wherein each altered polypeptide contains at least one engineered cysteine residues at any of the EU positions described supra. In a more specific embodiment, said two or more altered polypeptides are the same.

In an exemplary embodiment, q is 1, m is 1, n is 2, Pro₁ is an altered CH3-containing binding polypeptide of the invention described supra, and Pro₂ is an altered CH1-containing binding polypeptide of the invention as described supra. In a more specific embodiment, said CH1-containing binding polypeptide is a Fab fragment or CH1-scFv fragment. In one embodiment, the altered CH3-containing binding polypeptide has at least one binding specificity for TRAIL-R2 and the altered CH1-containing binding polypeptide has at least one binding specificity for LTβR.

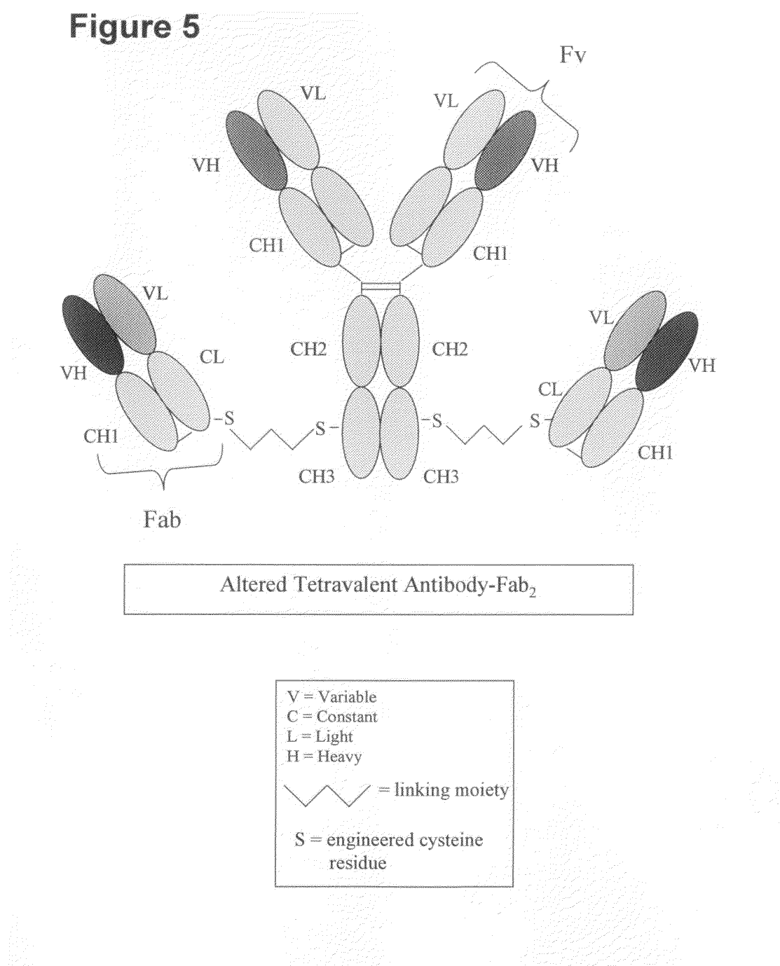

In another exemplary embodiment, q is 1, m is 1, n is 2, Pro₁ is an altered CH3-containing binding polypeptide of the invention described supra, and Pro₂ is an altered CL-containing binding polypeptide of the invention as described supra. In a more specific embodiment, said altered CL-containing binding polypeptide is a Fab fragment (see FIG. 5) or a CH1-scFv fragment. In one embodiment, the altered CH3-containing binding polypeptide has at least one binding specificity for TRAIL-R2 and the altered CL-containing binding polypeptide has at least one binding specificity for LTβR.

In another embodiment, m is 1, 2, 3, or 4 and Pro₁ is an altered CH1-containing binding polypeptide of the invention as described supra. In another embodiment, m is 1, 2, 3, or 4, Pro₁ is an altered CH₁-containing biding protein of the invention as described supra, and each Pro₂ is either a CH1-containing binding polypeptide as described supra, or a CL-containing binding polypeptide as described supra.

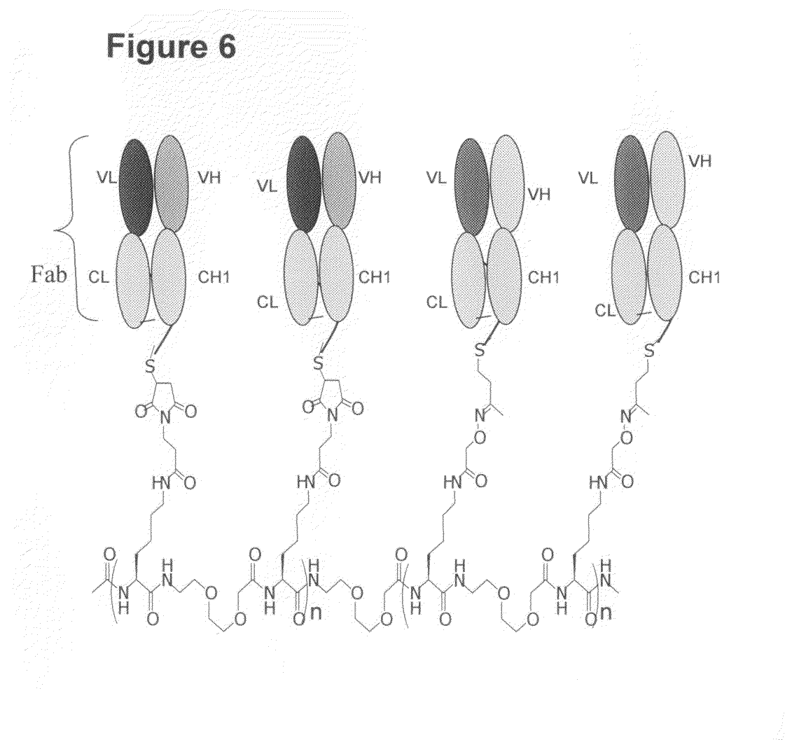

In an exemplary embodiment, q is 1, m is 3, n is 1, Pro₁ is an altered CH1-containing binding polypeptide of the invention described supra, and each Pro₂ is an altered CH1-containing binding polypeptide of the invention as described supra. In a more specific embodiment, each CH1-containing binding polypeptide is a Fab fragment (see FIG. 6) or CH1-scFv fragment. In one embodiment, at least one CH1-containing binding polypeptide has at least one binding specificity for TRAIL-R2. In another embodiment, at least one CH1-containing binding polypeptide has at least one binding specificity for LTβR. In one embodiment, at least one CH1-containing binding polypeptide has at least one binding specificity for TRAIL-R2 and at least one CH1-containing binding polypeptide has at least one binding specificity for LTβR.

In another embodiment, m is 1, 2, 3, or 4 and Pro₁ is an altered CL-containing binding polypeptide of the invention as described supra. In another embodiment, m is 1, 2, 3, or 4, Pro₁ is an altered CL-containing biding protein of the invention as described supra, and each Pro₂ is either a CH1-containing binding polypeptide as described supra, or a CL-containing binding polypeptide as described supra.

In an exemplary embodiment, q is 1, m is 3, n is 1, Pro₁ is an altered CL-containing binding polypeptide of the invention described supra, and each Pro₂ is an altered CL-containing binding polypeptide of the invention as described supra. In a more specific embodiment, each CL-containing binding polypeptide is a Fab fragment or CH1-scFv fragment. In a more specific embodiment, each CL-containing binding polypeptide is a Fab fragment or CL-scFv fragment. In one embodiment, at least one CL-containing binding polypeptide has at least one binding specificity for TRAIL-R2. In another embodiment, at least one CL-containing binding polypeptide has at least one binding specificity for LTβR. In one embodiment, at least one CL-containing binding polypeptide has at least one binding specificity for TRAIL-R2 and at least one CH1-containing binding polypeptide has at least one binding specificity for LTβR.

In a further embodiment, the invention provides a modified binding protein of the formula (VI):

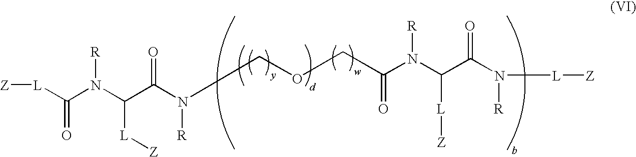

wherein:

L is an independently selected linker moiety for each occurrence or B when Z is an altered binding polypeptide, Pro-S—;

B is a bridging moiety;

R is selected independently for each occurrence from the group consisting of alkyl, alkenyl, alkynyl, acyl, and hydrogen;

Z is an independently selected effector moiety (E), affinity moiety, PEGylation moiety, hydrogen, amino acid side chain moiety, or Pro-S— for each occurrence;

w and y are each independently selected positive integers for each occurrence;

Pro is an altered binding polypeptide;

S is a thioether bond; and

b and d are each independently selected for each occurrence from integers greater than 1, provided that at least one Z is Pro-S— and at least one Z is E, and pharmaceutically acceptable salts, esters, and prodrugs thereof.

In a further embodiment, Z is an affinity moiety. Affinity moieties include moieties which would allow the separation of an affinity moiety labeled binding protein of the invention from a mixture by contacting the affinity moiety with an affinity matrix. Examples of affinity moieties include, but are not limited to, biotin.

In a further embodiment, Z is a PEGylation moiety. PEGylation moieties generally increase the apparent molecule weight, and pharmacokinetic properties of the modified binding protein including clearance rate or half-life of the modified binding protein. PEGylation can also decrease antigenicity and immunogenicity of the modified binding protein. In addition, PEGylation can increase the solubility of a biologically-active molecule.

PEGylation may be carried out by any of the PEGylation reactions known in the art (e.g., Focus on Growth Factors 3:4-10 (1992); EP 0 154 316; and EP 0 401 384). Methods for preparing PEGylated modified binding proteins of the invention will generally comprise the steps of a) reacting the modified fragment with a polyethylene glycol molecule, such as a reactive ester or aldehyde derivative of polyethylene glycol, under conditions whereby one or more polyethylene glycol groups are attached, and b) obtaining the reaction products. It will be apparent to one of ordinary skill in the art to select the optimal reaction conditions or the acylation reactions based on known parameters and the desired result. Exemplary pegylation moieties (or related polymers) include, for example, polyethylene glycol (“PEG”), polypropylene glycol (“PPG”), polyoxyethylated glycerol (“POG”) and other polyoxyethylated polyols, polyvinyl alcohol (“PVA) and other polyalkylene oxides, polyoxyethylated sorbitol, or polyoxyethylated glucose. The polymer can be a homopolymer, a random or block copolymer, a terpolymer based on the monomers listed above, straight chain or branched, substituted or unsubstituted as long as it has at least one active sulfone moiety. The polymeric portion can be of any length or molecular weight but these characteristics can affect the biological properties. Polymer average molecular weights particularly useful for decreasing clearance rates in pharmaceutical applications are in the range of 2,000 to 35,000 daltons. In addition, if two groups are linked to the polymer, one at each end, the length of the polymer can impact upon the effective distance, and other spatial relationships, between the two groups. Thus, one skilled in the art can vary the length of the polymer to optimize or confer the desired biological activity.

In another further embodiment, Z is the side chain of a natural or unnatural amino acid. In certain embodiments, Z may be a proline side chain and linked to an adjacent R group to form a ring. Examples of side chains include, for example, those of alanine, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine.

Examples of bridging moieties (B) include moieties which link Pro-S to the effector molecule (E). The bridging moeity may be attached to altered polypeptide through a disulfide linkage or thioether linkage. The bridging moiety may comprise a linker moiety comprising an optionally substituted chain of covalently atoms (which may be straight chain, cyclic, or branched.) In one embodiment, the bridging moiety comprises about 0 to about 50 carbon, oxygen, nitrogen, and sulfur atoms, optionally substituted with hydrogens or other substituents which allow the compound of the invention to perform its intended function. The bridging moiety may be formed by the reaction of an linker moiety with an altered polypeptide of the invention.

In one embodiment, the bridging moiety is attached to the altered binding polypeptide of the invention through a disulfide linkage or thioether linkage. In one embodiment, the engineered cysteine residue with which the disulfide linkage is formed is an exterior cysteine (e.g., a cysteine which does not form a disulfide bridge in the unmodified form of the polypeptide. In another embodiment, the bridging moiety is linked to a cysteine which does form disulfide bridges in the unmodified form of the polypeptide. In certain embodiments, the bridging moiety is linked to the polypeptide by cleaving one or more disulfide bonds in the polypeptide. The bridging moiety may also comprise urea, thioesters, etc. functional groups.

In other embodiments, the bridging moieties are attached to the binding polypeptides through carboxylate or amino groups of the polypeptide.

In a further embodiment, a portion of the modified binding protein of the invention is of the formula (VII):

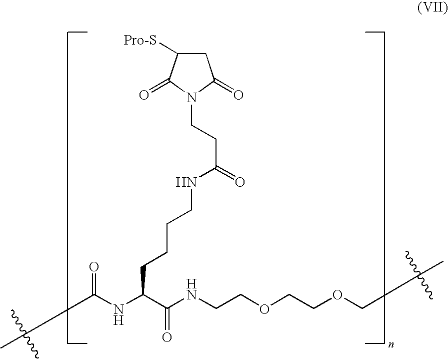

wherein

Pro is an altered binding polypeptide;

S is a thioether bond; and

n is 1, 2, 3, or 4.

In a further embodiment, a portion of the modified binding protein of the invention is of the formula (VIII):

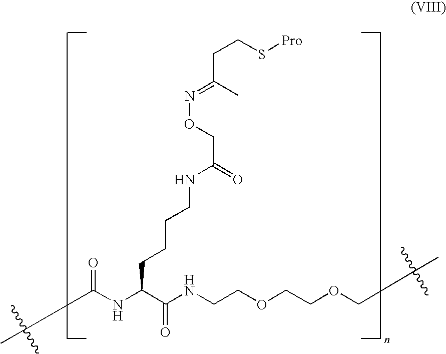

wherein

Pro is an altered binding polypeptide;

S is a thioether bond; and

n is 1, 2, 3, or 4.

### IX METHODS OF TREATMENT WITH MODIFIED BINDING POLYPEPTIDES OF THE INVENTION

The modified binding polypeptides of the invention can be used for diagnostic or therapeutic purposes. Preferred embodiments of the present invention provide kits and methods for the diagnosis and/or treatment of disorders, e.g., neoplastic disorders in a mammalian subject in need of such treatment. Preferably, the subject is a human.

The modified binding polypeptides of the instant invention will be useful in a number of different applications. For example, in one embodiment, the subject modified antibodies should be useful for reducing or eliminating cells bearing an epitope recognized by the antigen binding domain of the antibody. In another embodiment, the subject modified antibodies are effective in reducing the concentration of or eliminating soluble antigen in the circulation.

A. Anti-Tumor Therapy

In one embodiment, the therapeutic modified antibodies may reduce tumor size, inhibit tumor growth and/or prolong the survival time of tumor-bearing animals. Accordingly, this invention also relates to a method of treating tumors in a human or other animal by administering to such human or animal an effective, non-toxic amount of modified antibody. One skilled in the art would be able, by routine experimentation, to determine what an effective, non-toxic amount of modified binding polypeptide would be for the purpose of treating malignancies. For example, a therapeutically active amount of a modified binding polypeptide may vary according to factors such as the disease stage (e.g., stage I versus stage IV), age, sex, medical complications (e.g., immunosuppressed conditions or diseases) and weight of the subject, and the ability of the modified antibody to elicit a desired response in the subject. The dosage regimen may be adjusted to provide the optimum therapeutic response. For example, several divided doses may be administered daily, or the dose may be proportionally reduced as indicated by the exigencies of the therapeutic situation. Generally, however, an effective dosage is expected to be in the range of about 0.05 to 100 milligrams per kilogram body weight per day and more preferably from about 0.5 to 10, milligrams per kilogram body weight per day.

In general, the disclosed invention may be used to prophylactically or therapeutically treat any neoplasm comprising an antigenic marker that allows for the targeting of the cancerous cells by the modified antibody. Exemplary cancers that may be treated include, but are not limited to, prostate, gastric carcinomas such as colon, skin, breast, ovarian, lung and pancreatic. More particularly, the modified antibodies of the instant invention may be used to treat Kaposi's sarcoma, CNS neoplasias (capillary hemangioblastomas, meningiomas and cerebral metastases), melanoma, gastrointestinal and renal sarcomas, rhabdomyosarcoma, glioblastoma (preferably glioblastoma multiforme), leiomyosarcoma, retinoblastoma, papillary cystadenocarcinoma of the ovary, Wilm's tumor or small cell lung carcinoma. It will be appreciated that appropriate target binding polypeptides may be derived for tumor associated antigens related to each of the forgoing neoplasias without undue experimentation in view of the instant disclosure.

Exemplary hematologic malignancies that are amenable to treatment with the disclosed invention include Hodgkins and non-Hodgkins lymphoma as well as leukemias, including ALL-L3 (Burkitt's type leukemia), chronic lymphocytic leukemia (CLL) and monocytic cell leukemias. It will be appreciated that the compounds and methods of the present invention are particularly effective in treating a variety of B-cell lymphomas, including low grade/follicular non-Hodgkin's lymphoma (NHL), cell lymphoma (FCC), mantle cell lymphoma (MCL), diffuse large cell lymphoma (DLCL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia. It should be clear to those of skill in the art that these lymphomas will often have different names due to changing systems of classification, and that patients having lymphomas classified under different names may also benefit from the combined therapeutic regimens of the present invention. In addition to the aforementioned neoplastic disorders, it will be appreciated that the disclosed invention may advantageously be used to treat additional malignancies bearing compatible tumor associated antigens.

B. Immune Disorder Therapies

Besides neoplastic disorders, the modified binding polypeptides of the instant invention are particularly effective in the treatment of autoimmune disorders or abnormal immune responses. In this regard, it will be appreciated that the modified binding polypeptide of the present invention may be used to control, suppress, modulate or eliminate unwanted immune responses to both external and autoantigens. For example, in one embodiment, the antigen is an autoantigen. In another embodiment, the antigen is an allergan. In yet other embodiments, the antigen is an alloantigen or xenoantigen. Use of the disclosed modified binding polypeptides to reduce an immune response to alloantigens and xenoantigens is of particular use in transplantation, for example to inhibit rejection by a transplant recipient of a donor graft, e.g. a tissue or organ graft or bone marrow transplant. Additionally, suppression or elimination of donor T cells within a bone marrow graft is useful for inhibiting graft versus host disease.

In yet other embodiments the modified binding polypeptides of the present invention may be used to treat immune disorders that include, but are not limited to, allergic bronchopulmonary aspergillosis; Allergic rhinitis Autoimmune hemolytic anemia; Acanthosis nigricans; Allergic contact dermatitis; Addison's disease; Atopic dermatitis; Alopecia areata; Alopecia universalis; Amyloidosis; Anaphylactoid purpura; Anaphylactoid reaction; Aplastic anemia; Angio\edema, hereditary; Angioedema, idiopathic; Ankylosing spondylitis; Arteritis, cranial; Arteritis, giant cell; Arteritis, Takayasu's; Arteritis, temporal; Asthma; Ataxia-telangiectasia; Autoimmune oophoritis; Autoimmune orchitis; Autoimmune polyendocrine failure; Behcet's disease; Berger's disease; Buerger's disease; bronchitis; Bullous pemphigus; Candidiasis, chronic mucocutaneous; Caplan's syndrome; Post-myocardial infarction syndrome; Post-pericardiotomy syndrome; Carditis; Celiac sprue; Chagas's disease; Chediak-Higashi syndrome; Churg-Strauss disease; Cogan's syndrome; Cold agglutinin disease; CREST syndrome; Crohn's disease; Cryoglobulinemia; Cryptogenic fibrosing alveolitis; Dermatitis herpetifomis; Dermatomyositis; Diabetes mellitus; Diamond-Blackfan syndrome; DiGeorge syndrome; Discoid lupus erythematosus; Eosinophilic fasciitis; Episcleritis; Drythema elevatum diutinum; Erythema marginatum; Erythema multiforme; Erythema nodosum; Familial Mediterranean fever; Felty's syndrome; Fibrosis pulmonary; Glomerulonephritis, anaphylactoid; Glomerulonephritis, autoimmune; Glomerulonephritis, post-streptococcal; Glomerulonephritis, post-transplantation; Glomerulopathy, membranous; Goodpasture's syndrome; Granulocytopenia, immune-mediated; Granuloma annulare; Granulomatosis, allergic; Granulomatous myositis; Grave's disease; Hashimoto's thyroiditis; Hemolytic disease of the newborn; Hemochromatosis, idiopathic; Henoch-Schoenlein purpura; Hepatitis, chronic active and chronic progressive; Histiocytosis X; Hypereosinophilic syndrome; Idiopathic thrombocytopenic purpura; Job's syndrome; Juvenile dermatomyositis; Juvenile rheumatoid arthritis (Juvenile chronic arthritis); Kawasaki's disease; Keratitis; Keratoconjunctivitis sicca; Landry-Guillain-Barre-Strohl syndrome; Leprosy, lepromatous; Loeffler's syndrome; lupus; Lyell's syndrome; Lyme disease; Lymphomatoid granulomatosis; Mastocytosis, systemic; Mixed connective tissue disease; Mononeuritis multiplex; Muckle-Wells syndrome; Mucocutaneous lymph node syndrome; Mucocutaneous lymph node syndrome; Multicentric reticulohistiocytosis; Multiple sclerosis; Myasthenia gravis; Mycosis fungoides; Necrotizing vasculitis, systemic; Nephrotic syndrome; Overlap syndrome; Panniculitis; Paroxysmal cold hemoglobinuria; Paroxysmal nocturnal hemoglobinuria; Pemphigoid; Pemphigus; Pemphigus erythematosus; Pemphigus foliaceus; Pemphigus vulgaris; Pigeon breeder's disease; Pneumonitis, hypersensitivity; Polyarteritis nodosa; Polymyalgia rheumatic; Polymyositis; Polyneuritis, idiopathic; Portuguese familial polyneuropathies; Pre-eclampsia/eclampsia; Primary biliary cirrhosis; Progressive systemic sclerosis (Scleroderma); Psoriasis; Psoriatic arthritis; Pulmonary alveolar proteinosis; Pulmonary fibrosis, Raynaud's phenomenon/syndrome; Reidel's thyroiditis; Reiter's syndrome, Relapsing polychrondritis; Rheumatic fever; Rheumatoid arthritis; Sarcoidosis; Scleritis; Sclerosing cholangitis; Serum sickness; Sezary syndrome; Sjogren's syndrome; Stevens-Johnson syndrome; Still's disease; Subacute sclerosing panencephalitis; Sympathetic ophthalmia; Systemic lupus erythematosus; Transplant rejection; Ulcerative colitis; Undifferentiated connective tissue disease; Urticaria, chronic; Urticaria, cold; Uveitis; Vitiligo; Weber-Christian disease; Wegener's granulomatosis and Wiskott-Aldrich syndrome.

C. Anti-Inflammatory Therapy

In yet other embodiments, the modified binding polypeptides of the present invention may be used to treat inflammatory disorders that are caused, at least in part, or exacerbated by inflammation, e.g., increased blood flow, edema, activation of immune cells (e.g., proliferation, cytokine production, or enhanced phagocytosis). Exemplary inflammatory disorders include those in which inflammation or inflammatory factors (e.g., matrix metalloproteinases (MMPs), nitric oxide (NO), TNF, interleukins, plasma proteins, cellular defense systems, cytokines, lipid metabolites, proteases, toxic radicals, mitochondria, apoptosis, adhesion molecules, etc.) are involved or are present in an area in aberrant amounts, e.g., in amounts which may be advantageous to alter, e.g., to benefit the subject. The inflammatory process is the response of living tissue to damage. The cause of inflammation may be due to physical damage, chemical substances, micro-organisms, tissue necrosis, cancer or other agents. Acute inflammation is short-lasting, lasting only a few days. If it is longer lasting however, then it may be referred to as chronic inflammation.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they may last several weeks. The main characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or even longer, and are associated histologically with the presence of lymphocytes and macrophages and with proliferation of blood vessels and connective tissue. Recurrent inflammatory disorders include disorders which recur after a period of time or which have periodic episodes. Examples of recurrent inflammatory disorders include asthma and multiple sclerosis. Some disorders may fall within one or more categories.

Inflammatory disorders are generally characterized by heat, redness, swelling, pain and loss of function. Examples of causes of inflammatory disorders include, but are not limited to, microbial infections (e.g., bacterial, viral and fungal infections), physical agents (e.g., burns, radiation, and trauma), chemical agents (e.g., toxins and caustic substances), tissue necrosis and various types of immunologic reactions. Examples of inflammatory disorders include, but are not limited to, osteoarthritis, rheumatoid arthritis, acute and chronic infections (bacterial, viral and fungal); acute and chronic bronchitis, sinusitis, and other respiratory infections, including the common cold; acute and chronic gastroenteritis and colitis; acute and chronic cystitis and urethritis; acute respiratory distress syndrome; cystic fibrosis; acute and chronic dermatitis; acute and chronic conjunctivitis; acute and chronic serositis (pericarditis, peritonitis, synovitis, pleuritis and tendinitis); uremic pericarditis; acute and chronic cholecystis; acute and chronic vaginitis; acute and chronic uveitis; drug reactions; and burns (thermal, chemical, and electrical).

### X METHODS OF ADMINISTERING MODIFIED BINDING POLYPEPTIDES OF THE INVENTION

Methods of preparing and administering modified binding polypeptides of the invention to a subject are well known to or are readily determined by those skilled in the art. The route of administration of the modified binding polypeptide of the invention may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration are generally preferred. While all these forms of administration are clearly contemplated as being within the scope of the invention, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the modified binding polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a modified binding polypeptide by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture will preferably have labels or package inserts indicating that the associated compositions are useful for treating a subject suffering from, or predisposed to autoimmune or neoplastic disorders.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human but non-human mammals including transgenic mammals can also be treated. Treatment dosages may be titrated using routine methods known to those of skill in the art to optimize safety and efficacy.

For passive immunization with an antibody, the dosage can range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

Modified binding polypeptides of the invention can be administered on multiple occasions. Intervals between single dosages can be weekly, monthly or yearly. Intervals can also be irregular as indicated by measuring blood levels of modified binding polypeptide or antigen in the patient. In some methods, dosage is adjusted to achieve a plasma modified binding polypeptide concentration of 1-1000 μg/ml and in some methods 25-300 μg/ml. Alternatively, target binding polypeptides can be administered as a sustained release formulation, in which case less frequent administration is required. Dosage and frequency vary depending on the half-life of the antibody in the patient. In general, humanized antibodies show the longest half-life, followed by chimeric antibodies and nonhuman antibodies.

The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of antibody per dose, with dosages of from 5 to 25 mg being more commonly used for radioimmunoconjugates and higher doses for cytotoxin-drug modified antibodies) at relatively short intervals is sometimes required until progression of the disease is reduced or terminated, and preferably until the patient shows partial or complete amelioration of symptoms of disease. Thereafter, the patient can be administered a prophylactic regime.

Modified binding polypeptides of the invention can optionally be administered in combination with other agents that are effective in treating the disorder or condition in need of treatment (e.g., prophylactic or therapeutic).

Effective single treatment dosages (i.e., therapeutically effective amounts) of ⁹⁰Y-labeled modified binding polypeptides of the invention range from between about 5 and about 75 mCi, more preferably between about 10 and about 40 mCi. Effective single treatment non-marrow ablative dosages of ¹³¹I-modified antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-á-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While the modified binding polypeptides may be administered as described immediately above, it must be emphasized that in other embodiments modified binding polypeptides may be administered to otherwise healthy patients as a first line therapy. In such embodiments the modified binding polypeptides may be administered to patients having normal or average red marrow reserves and/or to patients that have not, and are not, undergoing. As used herein, the administration of modified binding polypeptides in conjunction or combination with an adjunct therapy means the sequential, simultaneous, coextensive, concurrent, concomitant or contemporaneous administration or application of the therapy and the disclosed antibodies. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates of the present invention. Conversely, cytotoxin associated modified binding polypeptides could be administered intravenously followed by tumor localized external beam radiation. In yet other embodiments, the modified binding polypeptide may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of the instant specification.

In this regard it will be appreciated that the combination of the modified binding polypeptide and the chemotherapeutic agent may be administered in any order and within any time frame that provides a therapeutic benefit to the patient. That is, the chemotherapeutic agent and modified binding polypeptide may be administered in any order or concurrently. In selected embodiments the modified binding polypeptides of the present invention will be administered to patients that have previously undergone chemotherapy. In yet other embodiments, the modified binding polypeptides and the chemotherapeutic treatment will be administered substantially simultaneously or concurrently. For example, the patient may be given the modified antibody while undergoing a course of chemotherapy. In preferred embodiments the modified antibody will be administered within 1 year of any chemotherapeutic agent or treatment. In other preferred embodiments the modified binding polypeptide will be administered within 10, 8, 6, 4, or 2 months of any chemotherapeutic agent or treatment. In still other preferred embodiments the modified binding polypeptide will be administered within 4, 3, 2 or 1 week of any chemotherapeutic agent or treatment. In yet other embodiments the modified binding polypeptide will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

It will further be appreciated that the modified binding polypeptides of the instant invention may be used in conjunction or combination with any chemotherapeutic agent or agents (e.g. to provide a combined therapeutic regimen) that eliminates, reduces, inhibits or controls the growth of neoplastic cells in vivo. Exemplary chemotherapeutic agents that are compatible with the instant invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in HARRISON'S PRINCIPLES OF INTERNAL MEDICINE 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more modified binding polypeptides of the invention as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

For patients with intermediate- and high-grade NHL, who fail to achieve remission or relapse, salvage therapy is used. Salvage therapies employ drugs such as cytosine arabinoside, carboplatin, cisplatin, etoposide and ifosfamide given alone or in combination. In relapsed or aggressive forms of certain neoplastic disorders the following protocols are often used: IMVP-16 (ifosfamide, methotrexate and etoposide), MIME (methyl-gag, ifosfamide, methotrexate and etoposide), DHAP (dexamethasone, high dose cytarabine and cisplatin), ESHAP (etoposide, methylpredisolone, HD cytarabine, cisplatin), CEPP(B) (cyclophosphamide, etoposide, procarbazine, prednisone and bleomycin) and CAMP (lomustine, mitoxantrone, cytarabine and prednisone) each with well known dosing rates and schedules.

The amount of chemotherapeutic agent to be used in combination with the modified binding polypeptides of the instant invention may vary by subject or may be administered according to what is known in the art. See for example, Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9^{th} ed. 1996).

As previously discussed, the modified binding polypeptides of the present invention, immunoreactive fragments or recombinants thereof may be administered in a pharmaceutically effective amount for the in vivo treatment of mammalian disorders. In this regard, it will be appreciated that the disclosed antibodies will be formulated to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of the modified binding polypeptide, immunoreactive fragment or recombinant thereof, conjugated or unconjugated to a therapeutic agent, shall be held to mean an amount sufficient to achieve effective binding to an antigen and to achieve a benefit, e.g., to ameliorate symptoms of a disease or disorder or to detect a substance or a cell. In the case of tumor cells, the modified binding polypeptide will be preferably be capable of interacting with selected immunoreactive antigens on neoplastic or immunoreactive cells and provide for an increase in the death of those cells. Of course, the pharmaceutical compositions of the present invention may be administered in single or multiple doses to provide for a pharmaceutically effective amount of the modified binding polypeptide.

In keeping with the scope of the present disclosure, the modified binding polypeptides of the invention may be administered to a human or other animal in accordance with the aforementioned methods of treatment in an amount sufficient to produce a therapeutic or prophylactic effect. The antibodies of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of modified binding polypeptides according to the present invention may prove to be particularly effective.

This invention is further illustrated by the following examples which should not be construed as limiting. The contents of all references, patents and published patent applications cited throughout this application are incorporated herein by reference.

### EXAMPLES

Throughout the examples, the following materials and methods were used unless otherwise stated.

### Materials and Methods

In general, the practice of the present invention employs, unless otherwise indicated, conventional techniques of chemistry, molecular biology, recombinant DNA technology, immunology (especially, e.g., antibody technology), and standard techniques in electrophoresis. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); and Current Protocols in Molecular Biology, eds. Ausubel et al., John Wiley & Sons (1992).

### Example 1

### Design of Altered Polypeptides Comprising Engineered Cysteines

### a) Design of Engineered Cysteine Residues in the CH3 Domain of Human IgG1:

The three dimensional structure of an human IgG1 Fc region was examined and amino acid positions or “sites” where surface-accessible cysteine residues could be engineered in the CH3 domain were evaluated by the following criteria: (1) the amino acid side-chain of the CH3 domain is exposed and solvent accessible; (2) the site is not required for binding of antigen, FcγR, FcRn, Protein A or Protein G; and (3) there is a low risk of disulfide formation with the counterpart cysteine residue in the other heavy chain of the symmetric Fc domain.

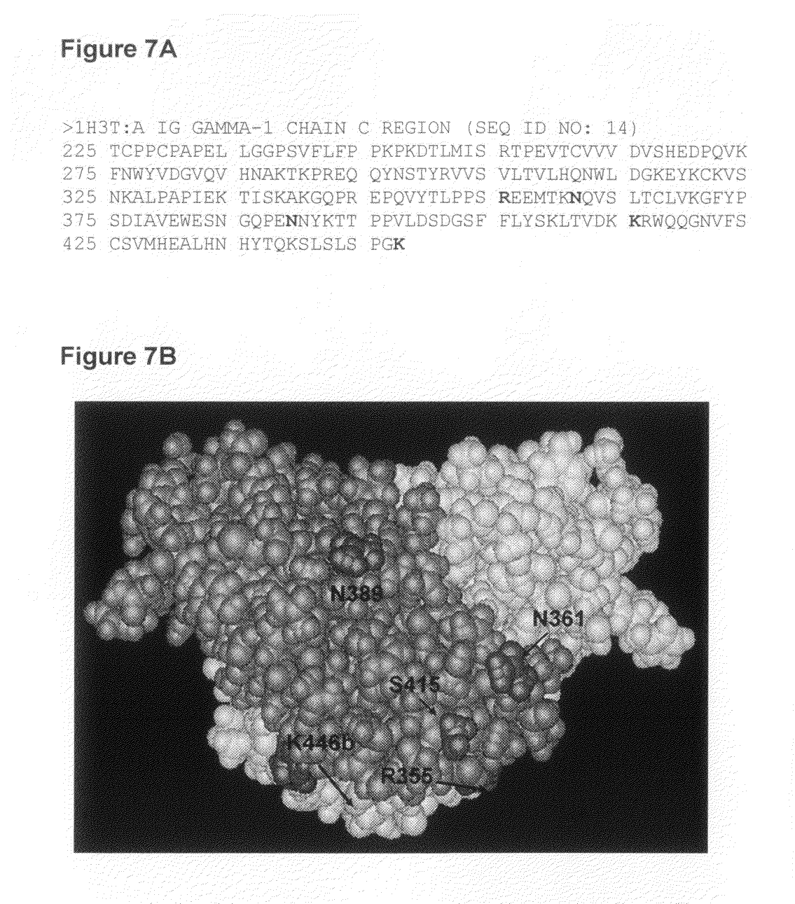

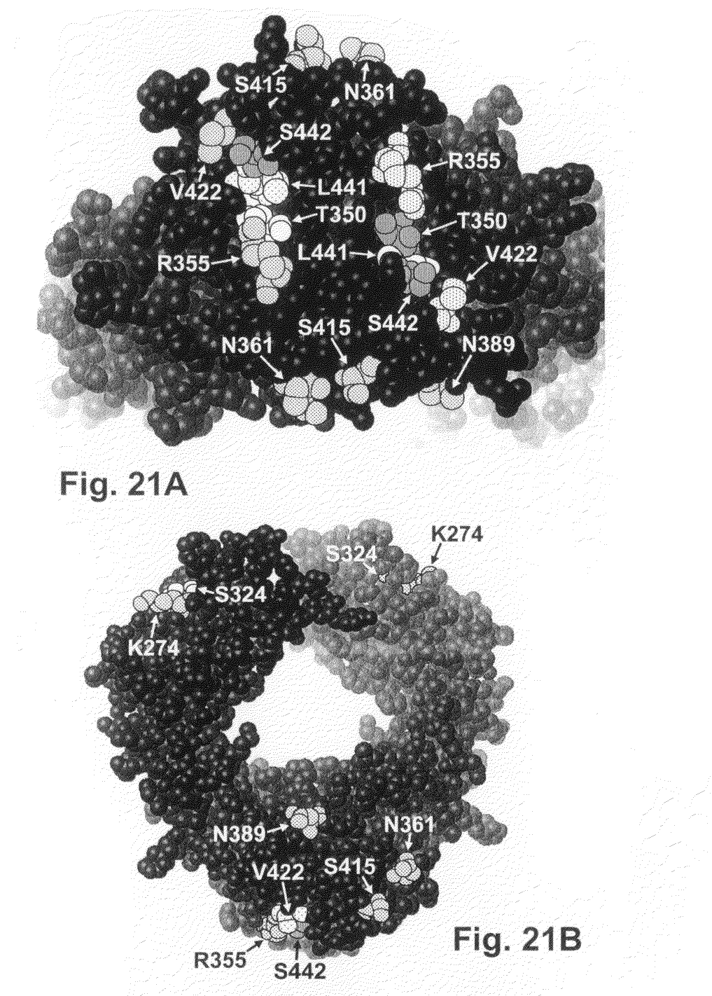

From a candidate list of R355, T359, K360, N361, N384, Q386, N389, D413, S415, Q418, V422, K439, S440, S442, L443, S444, and K 446b (EU numbering as in Kabat) a final list of 5 amino acid positions was chosen based on (1) compatibility of a cysteine residue with the backbone and sidechain configuration at that amino acid position in the three-dimensional IgG1 structure and (2) spatial separation of the amino acid positions to give 5 possible mutations in different locations on the surface of the CH3 domain. The final list comprised R355, N361, S415, N389, and K446b (the C-terminal residue of the Fc region, also referred to herein as K447). Each site was subjected to site-directed mutagenesis to construct R (original residue) 355C (engineered cysteine residue), N361C, S415C, N389C, and K446bC (K477C). The location of the 5 preferred sites in the amino acid sequence of a human IgG1 Fc region (SEQ ID NO: 14) is depicted in FIG. 7A (mutants sites are bolded). The location of the 5 preferred sites in a 3 dimensional model of the IgG1 Fc region is depicted in FIG. 7B. The location of additional preferred sites (K274, S324, T350, V422, L441, and S442) are depicted in FIGS. 21A and B.

### b) Design of Engineered Cysteine Residues in the CH1 or CL Domain of Human IgG1

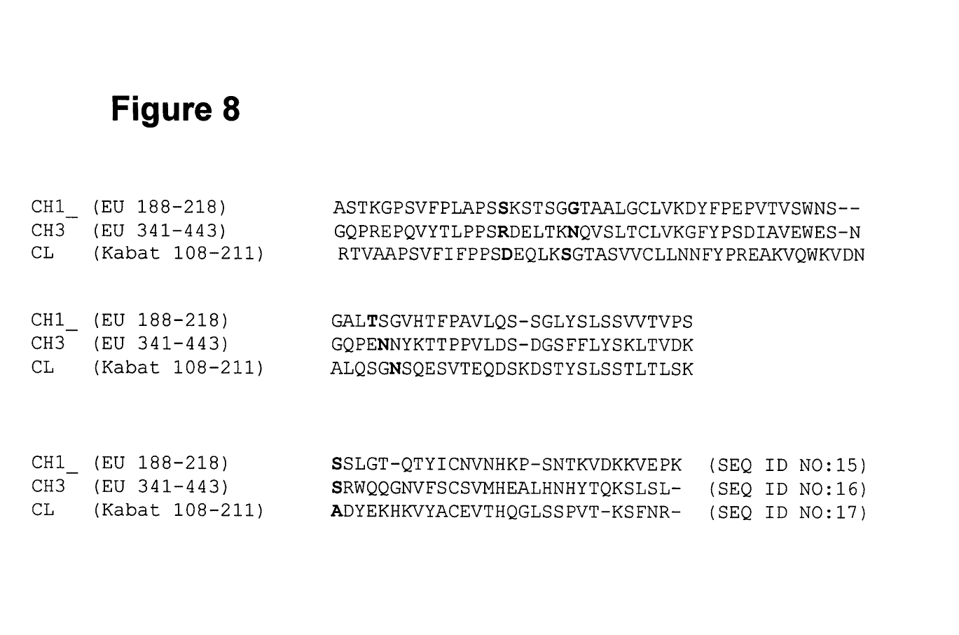

The CH1 and CH3 domains of immunoglobulins are homologous domains with very similar three-dimensional structures. The CH3 domain of an IgG1 antibody was structurally aligned with both the corresponding CH1 domain of IgG1 and CL domain of a human kappa light chain (see FIG. 8). Based on the sites for mutation in CH3 described above, corresponding residues in the CH1 and CL domains were selected. Those residues were evaluated for solvent exposure, and, if not solvent exposed, the nearest suitable solvent-exposed residue was chosen. Also, glycine residues with a disallowed phi/psi configuration were eliminated and the nearest suitable residue was selected. In the CH1 heavy chain EU numbered residues S132 (Kabat position 128), G138 (Kabat position 136), T164 (Kabat position 167), and S191 (Kabat position 196) were selected and in the CL light chain residues 122, 127, 158, and 184 (Kabat Numbering) were selected (see highlighted amino acid positions in FIG. 8).

### Example 2

### Production of Altered Antibodies Comprising Engineered Cysteines

### a) Production of an Altered Antibody Comprising Engineered Cysteine Residues in the CH3 Domain of Human IgG1:

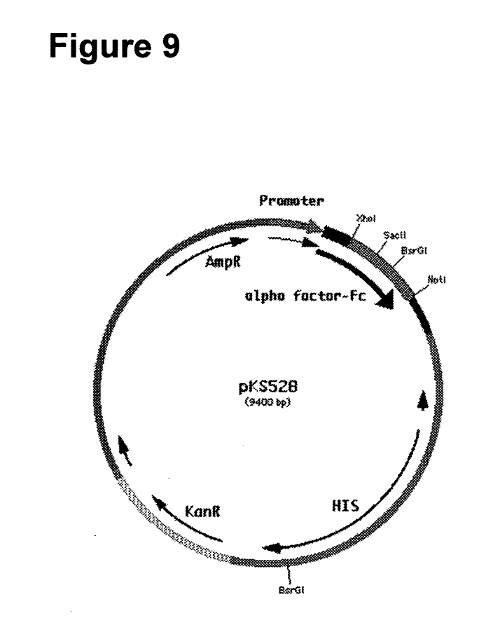

Using a yeast expression vector for a His6-Fc (human IgG1) protein, site directed mutagenesis with synthetic oligonucleotides was performed using the QuikChange mutagenesis kit of Stratagene (the 6×His tag is disclosed as SEQ ID NO: 59). The plasmid pKS528 was used a DNA template for mutagenesis (FIG. 9). pKS528 contains the Fc region of human IgG1 fused to the alpha factor secretion signal of Saccharomyces cerevisiae, with a His6 tag (SEQ ID NO: 59) at the N-terminus of the Fc region. This plasmid is an expression vector for Pichia pastoris. pK3528 also comprises a synthetic promoter having seven TetO repeats and the minimal AOX1 promoter of Pichia pastoris. The promoter can be induced with doxycyline in a P. pastoris strain expressing the reverse Tet transactivator (rTA). The P. pastoris strain MMC216 expresses rTA constitutively and is used for expression of coding sequences under control of this promoter.

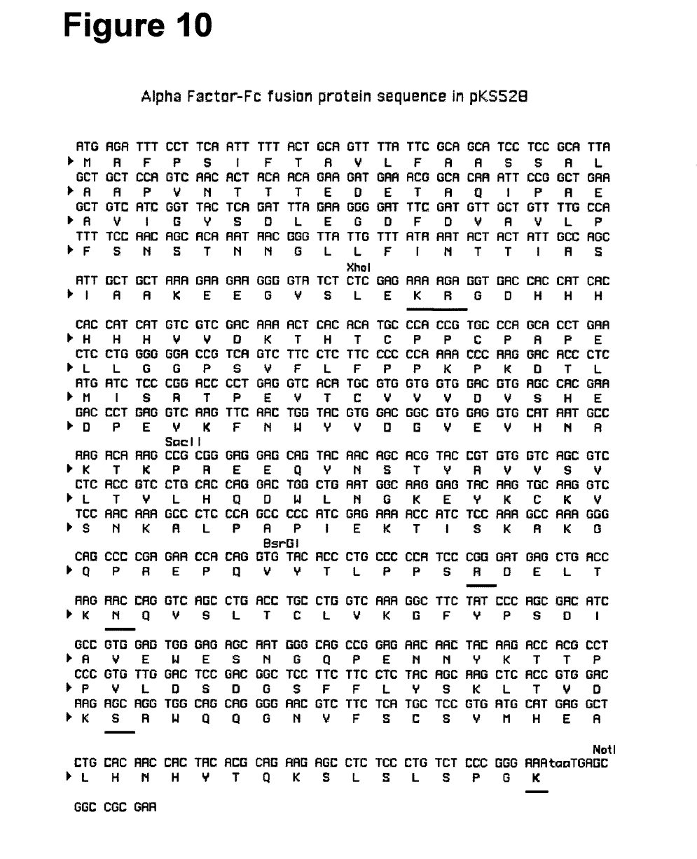

To construct an altered Fc polypeptide having one of 5 mutations at the selected amino acid positions in CH3 domain (R355C, N361C, N389C, S415C, and K446bC), site-directed mutagenesis by the QuikChange method of Stratagene was performed on the pKS528 plasmid DNA sequence with synthetic nucleotides as described in Table 2. The location of the target sites within nucleotide (SEQ ID NO: 18) and amino acid (SEQ ID NO: 19) sequences of the alpha-factor Fc-fusion protein sequence of pKS528 are depicted in FIG. 10.

TABLE 2

Oligonucleotides for Site-Directed Mutagenesis in the CH3 domain

| | |
|---|---|
| Mutation | |
| in CH3 | |
| region | Oligonucleotides |

| | | |
|---|---|---|
| R355C | 5′-GACCAAGAACCACTCGAGTAGCGTCCTACCCCCGTCCCAC-3′ | (SEQ ID NO: 28) |
| | 5′-GTGGGACGGGGGTAGGACGCTACTCGAGTGGTTCTTGGTC-3′ | (SEQ ID NO: 29) |
| N361C | 5′-CGGAAACTGGTCCGTTCAGTCCGACTGGACCGTGAACCAGTCGAG-3′ | (SEQ ID NO: 30) |
| | 5′-CTCGACTGGTTCACGGTCCAGTCGGACTGAACGGACCAGTTTCCG-3′ | (SEQ ID NO: 31) |
| N389C | 5′-GCACCAGAACATCAACGTGAGCCCGACGGGTAACGAGAG-3′ | (SEQ ID NO: 32) |
| | 5′-CTCTCGTTACCCGTCGGGCTCACGTTGATGTTCTGGTGC-3′ | (SEQ ID NO: 33) |
| S415C | 5′-GGGGACGACGGTGGACGTGAACAGGTGCCACTCG-3′ | (SEQ ID NO: 34) |
| | 5′-CGAGTGGCACCTGTTCACGTCCACCGTCGTCCCC-3′ | (SEQ ID NO: 35) |
| K446B-C | 5′-CGCCGGCGAGTAATCGTAGGCCCTCTGTCCCTCTC-3′ | (SEQ ID NO: 36) |
| | 5′-CGCCGGCGAGTAATCGTAGGCCCTCTGTCCCTCTC-3′ | (SEQ ID NO: 37) |

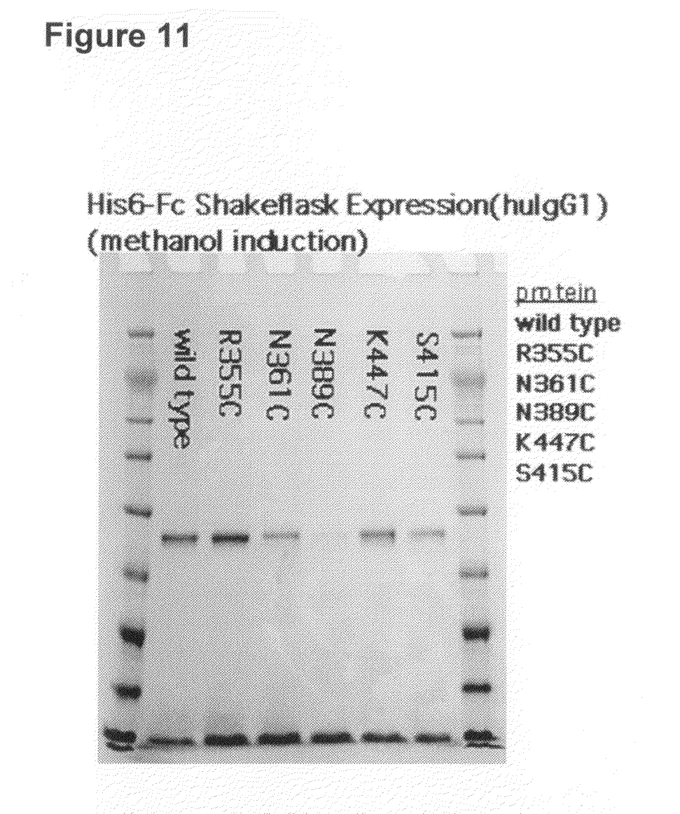

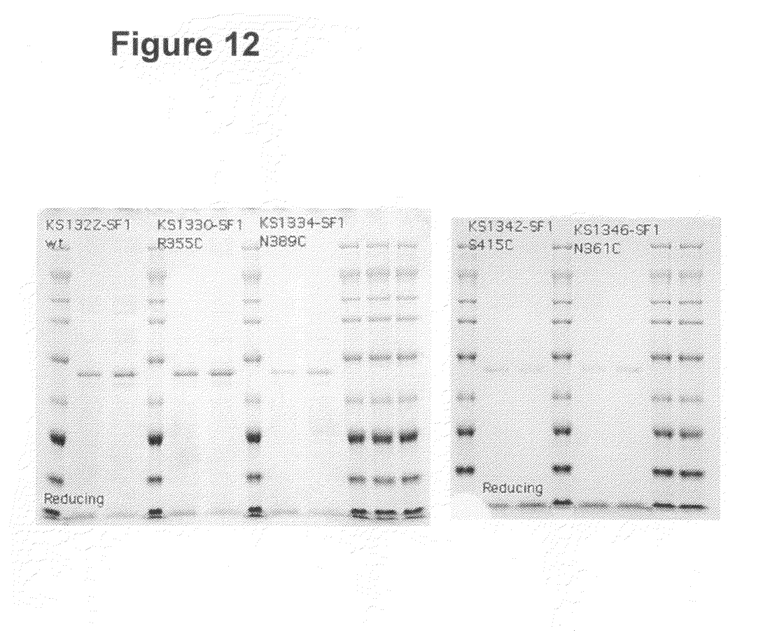

Candidate mutant plasmids were confirmed by DNA sequence analysis. Confirmed mutants were transformed to Pichia pastoris and protein expression was analyzed. For expression in Pichia, two different promoters were used. In one experiment, native AOX1 (induced by methanol) was used (see FIG. 11) and in a second experiment TetO-AOX1 (induced by doxycycline) was used (see FIG. 12). In the second experiment, cultures were grown at 25° C. in Fernbach shake flasks containing 500 mL BMGY medium and induced with 10 micrograms/mL Doxycycline. Unconcentrated culture supernatant was subjected to SDS-PAGE. The major protein in the culture medium is the Fc fragment (see FIG. 12). We observed that the R355C mutant expresses at levels similar to the wild-type in both experiments. The other mutants (N361C, S415C, N389C, and K446bC) express at somewhat reduced levels, but protein could be detected from all the mutants.

The DNA sequences for the 5 mutations may be subcloned to the Heavy Chain DNA of a full length antibody in a vector for protein expression in CHO cells.

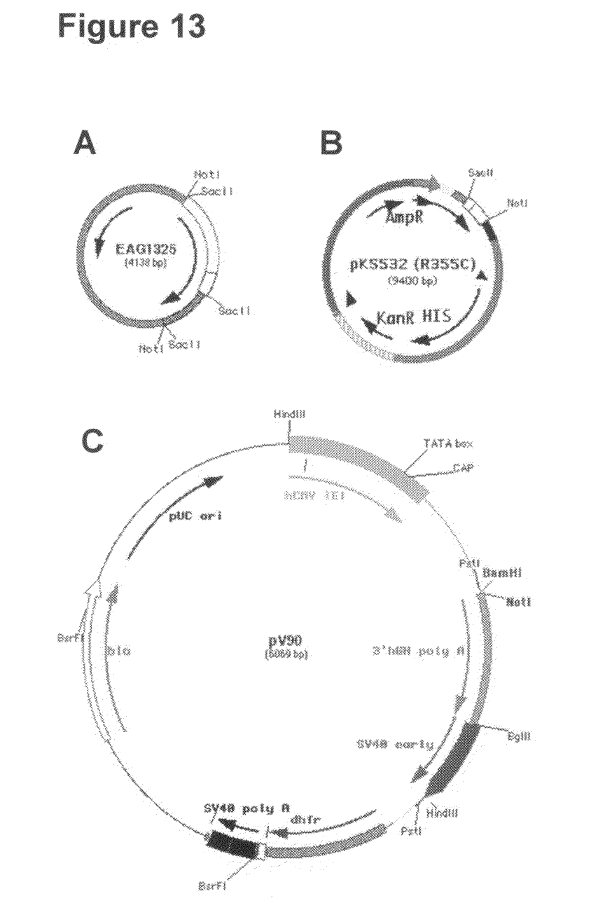

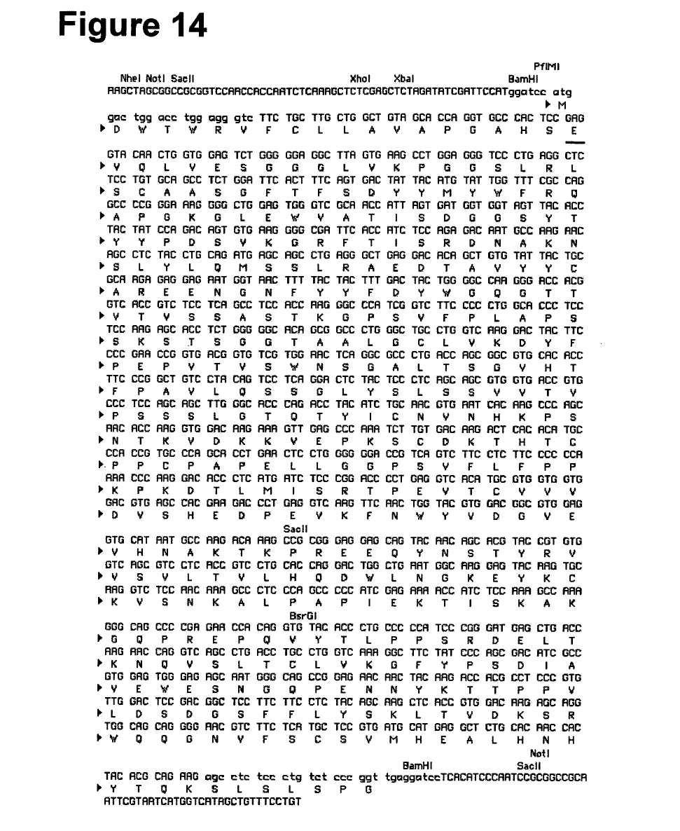

For example, FIG. 13 depicts plasmids which were used in a subcloning strategy for the generation of an altered humanized CBE11 antibody (an anti-Lymphotoxin Beta Receptor (anti-LTβR) monoclonal antibody). The VH—CH1-CH2 region of a CBE11 mAb Heavy Chain gene was subcloned as a NotI-SacII fragment from a EAG1325 plasmid (FIG. 13A). The coding region of the CBE11 Heavy Chain in the EAG1325 plasmid is depicted in FIG. 14 (amino acid sequence=SEQ ID NO: 20; nucleotide sequence=SEQ ID NO:21). Digestion with NotI prior to SacII destroys the SacII sites adjacent to the NotI site and leaves the internal SacII site as the only digestible site. An altered CH3 region (containing the R355C mutation) was also subcloned as a SacII-NotI fragment from pKS532 (FIG. 13B) or analogous plasmids for the other Cys mutations. Both fragments were inserted into the NotI site of PV90 (FIG. 13C) and candidates were screened by restriction digest for the appropriate orientation and confirmed by DNA sequence analysis. PV90 expression plasmids were cotransfected with a Light Chain expression vector and CHO cells expressing the desired altered CBE11 antibody were identified as described in Example 4 infra.

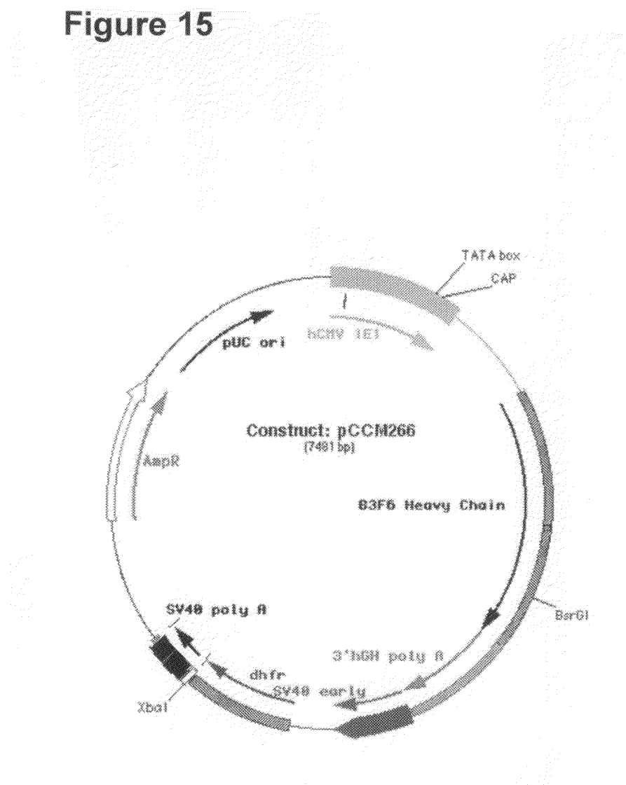

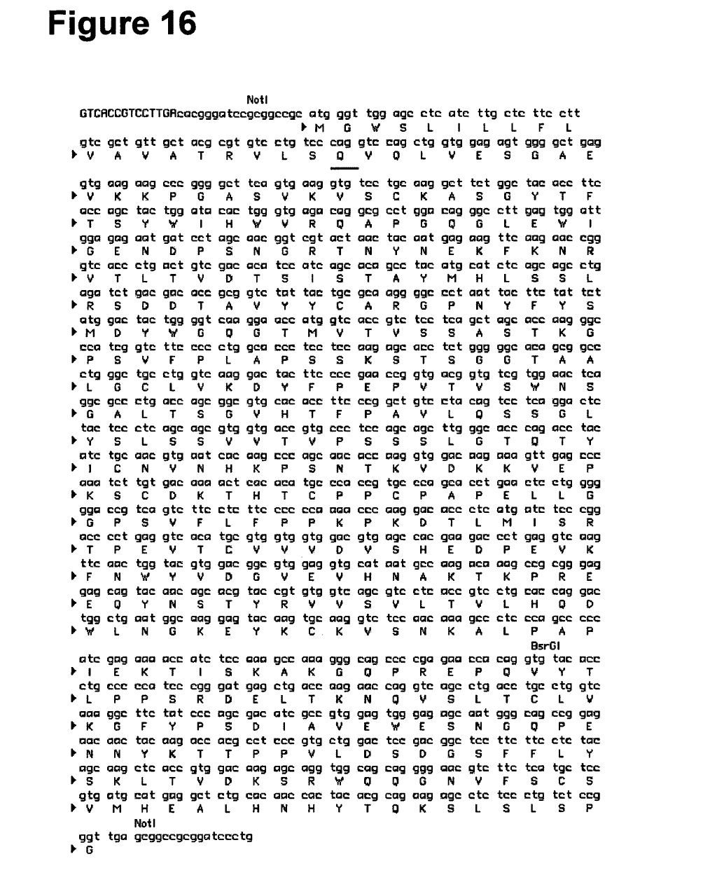

An altered CH3 domain having the R355C mutation was also introduced into humanized B3F6, an anti-CRIPTO monoclonal antibody. The VH—CH1-CH2 region of a B3F6 mAb Heavy Chain gene was subcloned from a pCCM266 plasmid (FIG. 15). The amino acid sequence (SEQ ID NO: 22) and nucleotide sequence (SEQ ID NO: 23) of the coding region of the B3F6 Heavy Chain in pMMC266 is depicted in FIG. 16. Following subcloning, the Heavy Chain sequences of altered B3F6 share identical sequences with pCCM266, except for the VH region and the particular Cys mutation. Consequently, substituting the BsrG1-Xba1 fragments of the CBE11-Cys mutants into the same sites of pCCM266 generates a set of Cys mutants in the B3F6 Heavy chain gene. As described in Example 4 infra, co-transfection of CHO cells with pCCM266 and an appropriate Light Chain expression vector generated CHO cells capable of expressing the desired altered B3F6 antibody.

### Example 3

### Production of altered Fab fragments comprising Engineered Cysteines

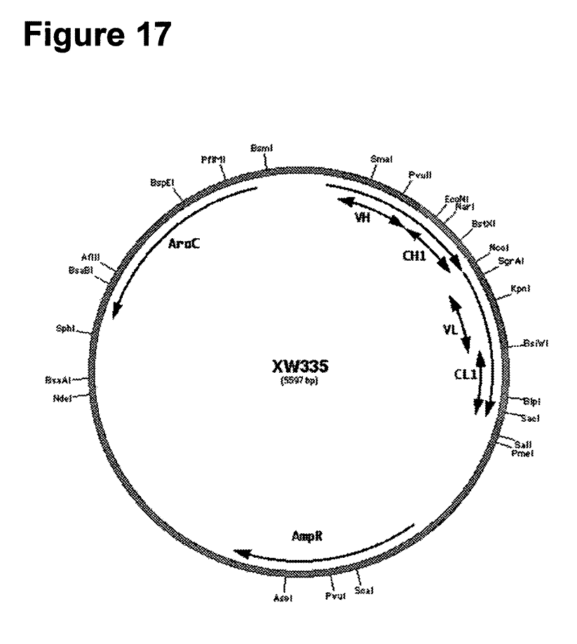

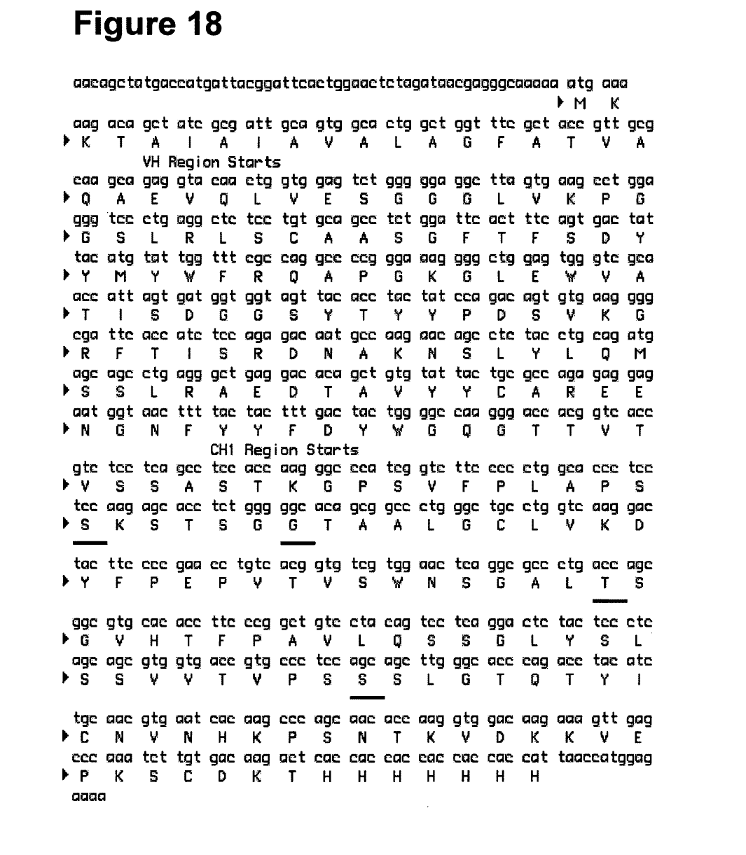

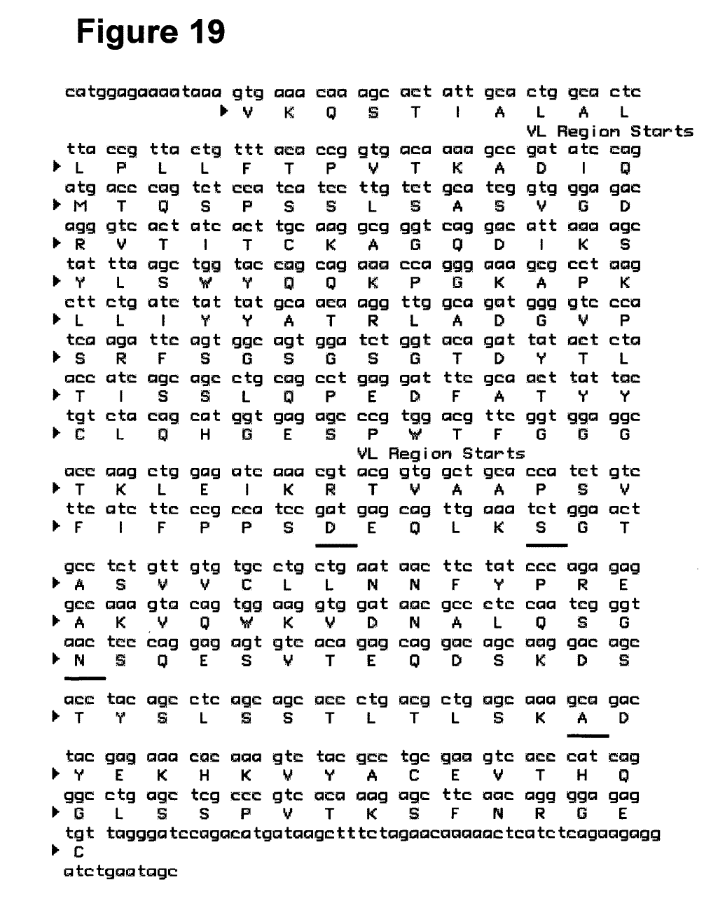

Site-directed mutagenesis CH1 and CL domains was carried out in using the E. coli expression plasmid XW335 as a template (FIG. 17). XW335 encodes both Heavy and Light chain regions for humanized CBE11 (anti-lymphotoxin beta receptor) and is engineered for expression of Fabs in E. coli. The araBAD promoter drives expression of the Heavy and Light chains of the Fab and the ompA signal sequence is present at the N-terminus of both the Heavy and Light chains and provides for secretion into the periplasm of E. coli. The amino acid (SEQ ID NO: 24) and nucleotide (SEQ ID NO: 25) sequences of the heavy chain of CBE11 Fab are depicted in FIG. 18. The amino acid (SEQ ID NO: 26) and nucleotide (SEQ ID NO: 27) sequences of the light chain of CBE11 Fab are depicted in FIG. 19. Mutagenesis by the QuikChange method (with oligonucleotides as shown in Table 3) was carried out and candidates were confirmed by DNA sequence analysis.

TABLE 3
Oligonucleotides for Site-Directed Mutagenesis in the CH1 and CL domain

| | | |
|---|---|---|
| Mutation in | | |
| CH1 region | | |
| S132-C | 5′-CGGGGGTCTCCACGAGAACGTTCTCCCTCGGTCCCCCTTCTG-3′ | (SEQ ID NO: 38) |
| | 5′-CAGAAGGGGGACCGAGGGAGAACGTTCTCGTGGAGACCCCCG-3′ | (SEQ ID NO: 39) |
| G138-C | 5′-CGTCGGGTCCCGGCGCCACGTGGGTCTCCACGAGAACC-3′ | (SEQ ID NO: 40) |
| | 5′-GCAGCCCAGGGCCGCGGTGCACCCAGAGGTGCTCTTGG-3′ | (SEQ ID NO: 41) |
| T164-C | 5′-CCACACGTGCGGCGACGTGTCCCGTGGACTCAAGGTGC-3′ | (SEQ ID NO: 42) |
| | 5′-GCACCTTGAGTCCACGGGACACGTCGCCGCACGTGTGG-3′ | (SEQ ID NO: 43) |
| S191-C | 5′-GACCCACGGGTTCGACGTCCTCCCGTGCCAGTG-3′ | (SEQ ID NO: 44) |
| | 5′-CACTGGCACGGGAGGACGTCGAACCCGTGGGTC-3′ | (SEQ ID NO: 45) |
| Mutation in | | |
| CKappa | | |
| region | | |
| D122-C | 5′-GGTCTAAAGTCGACGAGCGTCCTACCGCCCTTCTAC-3′ | (SEQ ID NO: 46) |
| | 5′-GTAGAAGGGCGGTAGGACGCTCGTCGACTTTAGACC-3′ | (SEQ ID NO: 47) |
| S127-C | 5′-GTTGTCTCCGTCAAGGCGTGAAGTCGACGAGTAGCCTACC-3′ | (SEQ ID NO: 48) |
| | 5′-GGTAGGCTACTCGTCGACTTCACGCCTTGACGGAGACAAC-3′ | (SEQ ID NO: 49) |
| N158-C | 5′-CTGTGAGAGGACCCTCGTTGGGCTGACGTCCCGCAATAGGTGG-3′ | (SEQ ID NO: 50) |
| | 5′-CCACCTATTGCGGGACGTCAGCCCAACGAGGGTCCTCTCACAG-3′ | (SEQ ID NO: 51) |
| A184-C | 5′-CACAAAGAGCATCAGCGTAAATGAGTCGCAGTCCCACG-3′ | (SEQ ID NO: 52) |
| | 5′-CGTGGGACTGCGACTCATTTACGCTGATGCTCTTTGTG-3′ | (SEQ ID NO: 53) |

### Example 4

### Production and Purification of Altered Antibodies Comprising Engineered Cysteines

To facilitate large-scale and stable production of altered CBE11 and anti-Cripto (B3F6) antibodies, CHO cells were transfected with a light chain expression plasmid and either of the PV90 and CCM266 expression plasmids described in Example 2 supra.

A variety of transfected cell lines were grown in one liter of conditioned medium and cell supernatants were subsequently collected and purified using Protein A affinity chromatography. Protein A eluates were then further purified by size exclusion chromatography (SEC). The yields (mg/L) from each protein purification experiment are summarized in Table 4. Most antibody constructs were expressed at yields of greater than 2 mg/L. In particular, altered antibodies containing the R355C and S415C mutations exhibited yields of greater than 5 mg/L in more than one cell line. Moreover, these antibodies exhibited a high percentage of homogeneous and aggregate-free species, as determined by SEC analysis. For example, an altered CBE11 antibody containing the R355C mutation exhibited a yield of 5.4 mg/L of aggregate-free protein after the size exclusion chromatography purification step, approximately 40% of the total protein recovered by Protein A purification.

TABLE 4
Yield of Altered Antibodies

| | | | Protein A | |
|---|---|---|---|---|
| Experiment | Altered Antibody | Cell Line | (mg/L) | SEC (mg/L) |
| 1 | CBE11, R355C | #1 | 12.6 | 5.4 |
| 2 | CBE11, R355C | DB11 | 6.5 | 1.6 |
| 3 | CBE11, N389C | AF9 | 0.45 | Not determined |
| 4 | CBE11, N389C | BC11 | 2.0 | Not determined |
| 5 | CBE11, N361C | AG7 | 2.8 | High M.W. |
| 6 | CBE11, N361C | AH3 | 3.3 | High M.W. |
| 7 | CRIPTO, R355C | | 4.8 | 1.6 |
| 8 | CRIPTO, S415C | AF6 | 7.6 | 1.1 |
| 9 | CRIPTO, S415C | BA10 | 10.8 | 4.2 |

### Example 5

### Production of Modified Antibodies by Site-Specific Conjugation

The altered antibodies of Example 4 were modified by site-specific conjugation of polyethylene glycol (PEG) residues to the engineered cysteine residues. However, the engineered cysteines were found to be blocked by cysteine adducts due to disulfide formation with free cysteines. Accordingly, the cysteine adducts were first removed by a 30 minute incubation with reductant (2 mM Mercaptoethylamine (MEA), pH 8) at room temperature. MEA was subsequently removed by gel filtration and the pH adjusted to 6.5. Conjugation of PEG moieties to the engineered cysteine residues was performed by reacting each antibody with a 5K molecular weight PEG-maleimide reagent (5K-PEGM). A low molecular weight maleimide (N-ethylmaleimide (NEM)) was employed as a control to show the oxidation state of the interchain H—H and H-L disulfides.

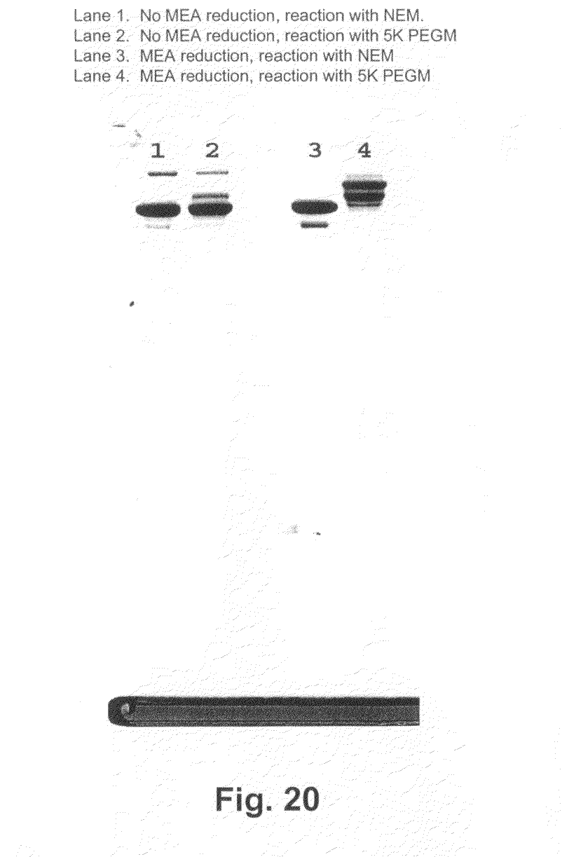

A denaturing, non-reducing SDS-PAGE gel of a modified anti-CRIPTO antibody (B3F6) comprising modifications of the engineered cysteine S415C is shown in FIG. 20. Without addition of MEA reductant, the unmodified antibody migrated primarily as one prominent band and two less prominent bands of higher and lower molecular weight (Lane 1). Reacting the unreduced antibody with 5K-PEGM resulted in a small, but detectable, level of antibodies of intermediate molecular weight (Lane 2), indicating that a small percentage of mAb had a free cysteine for conjugation with PEG. However, treatment with reducing agent prior to PEGylation (Lane 4) resulted in the appearance of a number of prominent higher molecular weight bands and disappearance of the single prominent band in lane 3. This result indicates that the majority of altered antibody was successfully modified with polymeric PEG moieties of various lengths.

```
<160> NUMBER OF SEQ ID NOS: 63
<210> SEQ ID NO 1
<211> LENGTH: 98
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 1
Ala Ser Thr Lys Gly Pro Ser Val Phe Pro Leu Ala Pro Ser Ser Lys
1 5 10 15
1 5 10 15
Ser Thr Ser Gly Gly Thr Ala Ala Leu Gly Cys Leu Val Lys Asp Tyr
20 25 30
                        20 25 30
Phe Pro Glu Pro Val Thr Val Ser Trp Asn Ser Gly Ala Leu Thr Ser
35 40 45
                35 40 45
Gly Val His Thr Phe Pro Ala Val Leu Gln Ser Ser Gly Leu Tyr Ser
50 55 60
        50 55 60
Leu Ser Ser Val Val Thr Val Pro Ser Ser Ser Leu Gly Thr Gln Thr
65 70 75 80
Tyr Ile Cys Asn Val Asn His Lys Pro Ser Asn Thr Lys Val Asp Lys
85 90 95
                                85 90 95
Lys Val
<210> SEQ ID NO 2
<211> LENGTH: 110
<212> TYPE: PRT
<213> ORGANISM: Artificial Sequence
<220> FEATURE:
<223> OTHER INFORMATION: Description of Artificial Sequence: Synthetic
            amino acid sequence
<220> FEATURE:
<221> NAME/KEY: MOD_RES
<222> LOCATION: (42)
<223> OTHER INFORMATION: Gln or Glu
<220> FEATURE:
<221> NAME/KEY: MOD_RES
<222> LOCATION: (53)
<223> OTHER INFORMATION: Gln or Glu
<220> FEATURE:
<221> NAME/KEY: MOD_RES
<222> LOCATION: (64)
<223> OTHER INFORMATION: Gln or Glu
<220> FEATURE:
<221> NAME/KEY: MOD_RES
<222> LOCATION: (82)
<223> OTHER INFORMATION: Asn or Asp
<220> FEATURE:
<221> NAME/KEY: MOD_RES
<222> LOCATION: (85)
<223> OTHER INFORMATION: Asp or Asn
<400> SEQUENCE: 2
Ala Pro Glu Leu Leu Gly Gly Pro Ser Val Phe Leu Phe Pro Pro Lys
1 5 10 15
1 5 10 15
Pro Lys Asp Thr Leu Met Ile Ser Arg Thr Pro Glu Val Thr Cys Val
20 25 30
                        20 25 30
Val Val Asp Val Ser His Glu Asp Pro Xaa Val Lys Phe Asn Trp Tyr
35 40 45
                35 40 45
Val Asp Gly Val Xaa Val His Asn Ala Lys Thr Lys Pro Arg Glu Xaa
50 55 60
        50 55 60
Gln Tyr Asn Ser Thr Tyr Arg Val Val Ser Val Leu Thr Val Leu His
65 70 75 80
Gln Xaa Trp Leu Xaa Gly Lys Glu Tyr Lys Cys Lys Val Ser Asn Lys
85 90 95
                                85 90 95
Ala Leu Pro Ala Pro Ile Glu Lys Thr Ile Ser Lys Ala Lys
100 105 110
                        100 105 110
<210> SEQ ID NO 3
<211> LENGTH: 110
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 3
Ala Pro Glu Leu Leu Gly Gly Pro Ser Val Phe Leu Phe Pro Pro Lys
1 5 10 15
1 5 10 15
Pro Lys Asp Thr Leu Met Ile Ser Arg Thr Pro Glu Val Thr Cys Val
20 25 30
                        20 25 30
Val Val Asp Val Ser His Glu Asp Pro Glu Val Lys Phe Asn Trp Tyr
35 40 45
                35 40 45
Val Asp Gly Val Glu Val His Asn Ala Lys Thr Lys Pro Arg Glu Glu
50 55 60
        50 55 60
Gln Tyr Asn Ser Thr Tyr Arg Val Val Ser Val Leu Thr Val Leu His
65 70 75 80
Gln Asp Trp Leu Asn Gly Lys Glu Tyr Lys Cys Lys Val Ser Asn Lys
85 90 95
                                85 90 95
Ala Leu Pro Ala Pro Ile Glu Lys Thr Ile Ser Lys Ala Lys
100 105 110
                        100 105 110
<210> SEQ ID NO 4
<211> LENGTH: 110
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 4
Ala Pro Glu Leu Leu Gly Gly Pro Ser Val Phe Leu Phe Pro Pro Lys
1 5 10 15
1 5 10 15
Pro Lys Asp Thr Leu Met Ile Ser Arg Thr Pro Glu Val Thr Cys Val
20 25 30
                        20 25 30
Val Val Asp Val Ser His Glu Asp Pro Gln Val Lys Phe Asn Trp Tyr
35 40 45
                35 40 45
Val Asp Gly Val Gln Val His Asn Ala Lys Thr Lys Pro Arg Glu Gln
50 55 60
        50 55 60
Gln Tyr Asn Ser Thr Tyr Arg Val Val Ser Val Leu Thr Val Leu His
65 70 75 80
Gln Asn Trp Leu Asp Gly Lys Glu Tyr Lys Cys Lys Val Ser Asn Lys
85 90 95
                                85 90 95
Ala Leu Pro Ala Pro Ile Glu Lys Thr Ile Ser Lys Ala Lys
100 105 110
                        100 105 110
<210> SEQ ID NO 5
<211> LENGTH: 106
<212> TYPE: PRT
<213> ORGANISM: Artificial Sequence
<220> FEATURE:
<223> OTHER INFORMATION: Description of Artificial Sequence: Synthetic
            amino acid sequence
<220> FEATURE:
<221> NAME/KEY: MOD_RES
<222> LOCATION: (16)
<223> OTHER INFORMATION: Glu or Asp
<220> FEATURE:
<221> NAME/KEY: MOD_RES
<222> LOCATION: (18)
<223> OTHER INFORMATION: Met or Leu
<400> SEQUENCE: 5
Gly Gln Pro Arg Glu Pro Gln Val Tyr Thr Leu Pro Pro Ser Arg Xaa
1 5 10 15
1 5 10 15
Glu Xaa Thr Lys Asn Gln Val Ser Leu Thr Cys Leu Val Lys Gly Phe
20 25 30
                        20 25 30
Tyr Pro Ser Asp Ile Ala Val Glu Trp Glu Ser Asn Gly Gln Pro Glu
35 40 45
                35 40 45
Asn Asn Tyr Lys Thr Thr Pro Pro Val Leu Asp Ser Asp Gly Ser Phe
50 55 60
        50 55 60
Phe Leu Tyr Ser Lys Leu Thr Val Asp Lys Ser Arg Trp Gln Gln Gly
65 70 75 80
Asn Val Phe Ser Cys Ser Val Met His Glu Ala Leu His Asn His Tyr
85 90 95
                                85 90 95
Thr Gln Lys Ser Leu Ser Leu Ser Pro Gly
100 105
                        100 105
<210> SEQ ID NO 6
<211> LENGTH: 107
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 6
Gly Gln Pro Arg Glu Pro Gln Val Tyr Thr Leu Pro Pro Ser Arg Asp
1 5 10 15
1 5 10 15
Glu Leu Thr Lys Asn Gln Val Ser Leu Thr Cys Leu Val Lys Gly Phe
20 25 30
                        20 25 30
Tyr Pro Ser Asp Ile Ala Val Glu Trp Glu Ser Asn Gly Gln Pro Glu
35 40 45
                35 40 45
Asn Asn Tyr Lys Thr Thr Pro Pro Val Leu Asp Ser Asp Gly Ser Phe
50 55 60
        50 55 60
Phe Leu Tyr Ser Lys Leu Thr Val Asp Lys Ser Arg Trp Gln Gln Gly
65 70 75 80
Asn Val Phe Ser Cys Ser Val Met His Glu Ala Leu His Asn His Tyr
85 90 95
                                85 90 95
Thr Gln Lys Ser Leu Ser Leu Ser Pro Gly Lys
100 105
                        100 105
<210> SEQ ID NO 7
<211> LENGTH: 107
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 7
Gly Gln Pro Arg Glu Pro Gln Val Tyr Thr Leu Pro Pro Ser Arg Glu
1 5 10 15
1 5 10 15
Glu Met Thr Lys Asn Gln Val Ser Leu Thr Cys Leu Val Lys Gly Phe
20 25 30
                        20 25 30
Tyr Pro Ser Asp Ile Ala Val Glu Trp Glu Ser Asn Gly Gln Pro Glu
35 40 45
                35 40 45
Asn Asn Tyr Lys Thr Thr Pro Pro Val Leu Asp Ser Asp Gly Ser Phe
50 55 60
        50 55 60
Phe Leu Tyr Ser Lys Leu Thr Val Asp Lys Ser Arg Trp Gln Gln Gly
65 70 75 80
Asn Val Phe Ser Cys Ser Val Met His Glu Ala Leu His Asn His Tyr
85 90 95
                                85 90 95
Thr Gln Lys Ser Leu Ser Leu Ser Pro Gly Lys
100 105
                        100 105
<210> SEQ ID NO 8
<211> LENGTH: 107
<212> TYPE: PRT
<213> ORGANISM: Mus musculus
<400> SEQUENCE: 8
Asp Ile Lys Met Thr Gln Ser Pro Ser Ser Met Tyr Ala Ser Leu Gly
1 5 10 15
1 5 10 15
Glu Arg Val Thr Ile Thr Cys Lys Ala Gly Gln Asp Ile Lys Ser Tyr
20 25 30
                        20 25 30
Leu Ser Trp Tyr Gln Gln Lys Pro Trp Lys Ser Pro Lys Ile Leu Ile
35 40 45
                35 40 45
Tyr Tyr Ala Thr Arg Leu Ala Asp Gly Val Pro Ser Arg Phe Ser Gly
50 55 60
        50 55 60
Ser Gly Ser Gly Gln Asp Tyr Ser Leu Thr Ile Ser Ser Leu Glu Ser
65 70 75 80
Asp Asp Thr Ala Thr Tyr Tyr Cys Leu Gln His Gly Glu Ser Pro Trp
85 90 95
                                85 90 95
Thr Phe Gly Gly Gly Thr Lys Leu Glu Ile Lys
100 105
                        100 105
<210> SEQ ID NO 9
<211> LENGTH: 120
<212> TYPE: PRT
<213> ORGANISM: Mus musculus
<400> SEQUENCE: 9
Glu Val Gln Leu Val Glu Ser Gly Gly Gly Leu Val Lys Pro Gly Gly
1 5 10 15
1 5 10 15
Ser Leu Lys Leu Ser Cys Ala Ala Ser Gly Phe Thr Phe Ser Asp Tyr
20 25 30
                        20 25 30
Tyr Met Tyr Trp Phe Arg Gln Thr Pro Glu Lys Arg Leu Glu Trp Val
35 40 45
                35 40 45
Ala Thr Ile Ser Asp Gly Gly Ser Tyr Thr Tyr Tyr Pro Asp Ser Val
50 55 60
        50 55 60
Lys Gly Arg Phe Thr Ile Ser Arg Asp Asn Ala Lys Asn Asn Leu Tyr
65 70 75 80
Leu Gln Met Ser Ser Leu Lys Ser Glu Asp Thr Ala Met Tyr Tyr Cys
85 90 95
                                85 90 95
Val Arg Glu Glu Asn Gly Asn Phe Tyr Tyr Phe Asp Tyr Trp Gly Gln
100 105 110
                        100 105 110
Gly Thr Thr Val Thr Val Ser Ser
115 120
                115 120
<210> SEQ ID NO 10
<211> LENGTH: 107
<212> TYPE: PRT
<213> ORGANISM: Artificial Sequence
<220> FEATURE:
<223> OTHER INFORMATION: Description of Artificial Sequence: Synthetic
            humanized antibody
<400> SEQUENCE: 10
Asp Ile Gln Met Thr Gln Ser Pro Ser Ser Leu Ser Ala Ser Val Gly
1 5 10 15
1 5 10 15
Asp Arg Val Thr Ile Thr Cys Lys Ala Gly Gln Asp Ile Lys Ser Tyr
20 25 30
                        20 25 30
Leu Ser Trp Tyr Gln Gln Lys Pro Gly Lys Ala Pro Lys Leu Leu Ile
35 40 45
                35 40 45
Tyr Tyr Ala Thr Arg Leu Ala Asp Gly Val Pro Ser Arg Phe Ser Gly
50 55 60
        50 55 60
Ser Gly Ser Gly Thr Asp Tyr Thr Leu Thr Ile Ser Ser Leu Gln Pro
65 70 75 80
Glu Asp Phe Ala Thr Tyr Tyr Cys Leu Gln His Gly Glu Ser Pro Trp
85 90 95
                                85 90 95
Thr Phe Gly Gly Gly Thr Lys Leu Glu Ile Lys
100 105
                        100 105
<210> SEQ ID NO 11
<211> LENGTH: 120
<212> TYPE: PRT
<213> ORGANISM: Artificial Sequence
<220> FEATURE:
<223> OTHER INFORMATION: Description of Artificial Sequence: Synthetic
            humanized antibody
<400> SEQUENCE: 11
Glu Val Gln Leu Val Glu Ser Gly Gly Gly Leu Val Lys Pro Gly Gly
1 5 10 15
1 5 10 15
Ser Leu Arg Leu Ser Cys Ala Ala Ser Gly Phe Thr Phe Ser Asp Tyr
20 25 30
                        20 25 30
Tyr Met Tyr Trp Phe Arg Gln Ala Pro Gly Lys Gly Leu Glu Trp Val
35 40 45
                35 40 45
Ala Thr Ile Ser Asp Gly Gly Ser Tyr Thr Tyr Tyr Pro Asp Ser Val
50 55 60
        50 55 60
Lys Gly Arg Phe Thr Ile Ser Arg Asp Asn Ala Lys Asn Ser Leu Tyr
65 70 75 80
Leu Gln Met Ser Ser Leu Arg Ala Glu Asp Thr Ala Val Tyr Tyr Cys
85 90 95
                                85 90 95
Ala Arg Glu Glu Asn Gly Asn Phe Tyr Tyr Phe Asp Tyr Trp Gly Gln
100 105 110
                        100 105 110
Gly Thr Thr Val Thr Val Ser Ser
115 120
<210> SEQ ID NO 12
<211> LENGTH: 115
<212> TYPE: PRT
<213> ORGANISM: Mus musculus
<400> SEQUENCE: 12
Gln Leu Val Leu Thr Gln Ser Ser Ser Val Ser Phe Ser Leu Gly Ala
1 5 10 15
1 5 10 15
Ser Ala Lys Leu Thr Cys Thr Leu Ser Ser Gln His Ser Thr Tyr Thr
20 25 30
                        20 25 30
Ile Glu Trp Tyr Gln Gln Gln Pro Leu Lys Pro Pro Lys Tyr Val Met
35 40 45
                35 40 45
Glu Leu Lys Lys Asp Gly Ser His Ser Thr Gly Asp Gly Ile Pro Asp
50 55 60
        50 55 60
Arg Phe Ser Gly Ser Ser Ser Gly Ala Asp Arg Tyr Leu Ser Ile Ser
65 70 75 80
Asn Ile Gln Pro Glu Asp Glu Ala Ile Tyr Ile Cys Gly Val Gly Asp
85 90 95
                                85 90 95
Thr Ile Lys Glu Gln Phe Val Tyr Val Phe Gly Gly Gly Thr Lys Val
100 105 110
                        100 105 110
Glu Ile Lys
115
                115
<210> SEQ ID NO 13
<211> LENGTH: 120
<212> TYPE: PRT
<213> ORGANISM: Mus musculus
<400> SEQUENCE: 13
Gln Ile Gln Leu Val Gln Ser Gly Pro Glu Leu Lys Lys Pro Gly Glu
1 5 10 15
1 5 10 15
Thr Val Lys Ile Ser Cys Lys Ala Ser Gly Phe Thr Phe Thr Asp Tyr
20 25 30
                        20 25 30
Ser Ile His Trp Val Lys Gln Ala Pro Gly Lys Gly Leu Lys Trp Met
35 40 45
                35 40 45
Gly Trp Ile Asn Thr Glu Thr Gly Glu Pro Thr Tyr Thr Asp Asp Phe
50 55 60
        50 55 60
Lys Gly Arg Phe Ala Phe Ser Leu Val Thr Ser Ala Thr Thr Ala Tyr
65 70 75 80
Leu Gln Ile Asn Asn Leu Asn Asn Glu Asp Thr Ala Thr Phe Phe Cys
85 90 95
                                85 90 95
Ala Arg Phe Ile Tyr Asp Pro Tyr Trp Gly Phe Ala Tyr Trp Gly Gln
100 105 110
                        100 105 110
Gly Thr Leu Val Thr Val Ser Ala
115 120
                115 120
<210> SEQ ID NO 14
<211> LENGTH: 223
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 14
Thr Cys Pro Pro Cys Pro Ala Pro Glu Leu Leu Gly Gly Pro Ser Val
1 5 10 15
1 5 10 15
Phe Leu Phe Pro Pro Lys Pro Lys Asp Thr Leu Met Ile Ser Arg Thr
20 25 30
                        20 25 30
Pro Glu Val Thr Cys Val Val Val Asp Val Ser His Glu Asp Pro Gln
35 40 45
                35 40 45
Val Lys Phe Asn Trp Tyr Val Asp Gly Val Gln Val His Asn Ala Lys
50 55 60
        50 55 60
Thr Lys Pro Arg Glu Gln Gln Tyr Asn Ser Thr Tyr Arg Val Val Ser
65 70 75 80
Val Leu Thr Val Leu His Gln Asn Trp Leu Asp Gly Lys Glu Tyr Lys
85 90 95
                                85 90 95
Cys Lys Val Ser Asn Lys Ala Leu Pro Ala Pro Ile Glu Lys Thr Ile
100 105 110
                        100 105 110
Ser Lys Ala Lys Gly Gln Pro Arg Glu Pro Gln Val Tyr Thr Leu Pro
115 120 125
                115 120 125
Pro Ser Arg Glu Glu Met Thr Lys Asn Gln Val Ser Leu Thr Cys Leu
130 135 140
        130 135 140
Val Lys Gly Phe Tyr Pro Ser Asp Ile Ala Val Glu Trp Glu Ser Asn
145 150 155 160
Gly Gln Pro Glu Asn Asn Tyr Lys Thr Thr Pro Pro Val Leu Asp Ser
165 170 175
                                165 170 175
Asp Gly Ser Phe Phe Leu Tyr Ser Lys Leu Thr Val Asp Lys Lys Arg
180 185 190
                        180 185 190
Trp Gln Gln Gly Asn Val Phe Ser Cys Ser Val Met His Glu Ala Leu
195 200 205
                195 200 205
His Asn His Tyr Thr Gln Lys Ser Leu Ser Leu Ser Pro Gly Lys
210 215 220
        210 215 220
<210> SEQ ID NO 15
<211> LENGTH: 101
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 15
Ala Ser Thr Lys Gly Pro Ser Val Phe Pro Leu Ala Pro Ser Ser Lys
1 5 10 15
1 5 10 15
Ser Thr Ser Gly Gly Thr Ala Ala Leu Gly Cys Leu Val Lys Asp Tyr
20 25 30
                        20 25 30
Phe Pro Glu Pro Val Thr Val Ser Trp Asn Ser Gly Ala Leu Thr Ser
35 40 45
                35 40 45
Gly Val His Thr Phe Pro Ala Val Leu Gln Ser Ser Gly Leu Tyr Ser
50 55 60
        50 55 60
Leu Ser Ser Val Val Thr Val Pro Ser Ser Ser Leu Gly Thr Gln Thr
65 70 75 80
Tyr Ile Cys Asn Val Asn His Lys Pro Ser Asn Thr Lys Val Asp Lys
85 90 95
                                85 90 95
Lys Val Glu Pro Lys
100
                        100
<210> SEQ ID NO 16
<211> LENGTH: 103
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 16
Gly Gln Pro Arg Glu Pro Gln Val Tyr Thr Leu Pro Pro Ser Arg Asp
1 5 10 15
1 5 10 15
Glu Leu Thr Lys Asn Gln Val Ser Leu Thr Cys Leu Val Lys Gly Phe
20 25 30
                        20 25 30
Tyr Pro Ser Asp Ile Ala Val Glu Trp Glu Ser Asn Gly Gln Pro Glu
35 40 45
                35 40 45
Asn Asn Tyr Lys Thr Thr Pro Pro Val Leu Asp Ser Asp Gly Ser Phe
50 55 60
        50 55 60
Phe Leu Tyr Ser Lys Leu Thr Val Asp Lys Ser Arg Trp Gln Gln Gly
65 70 75 80
Asn Val Phe Ser Cys Ser Val Met His Glu Ala Leu His Asn His Tyr
85 90 95
                                85 90 95
Thr Gln Lys Ser Leu Ser Leu
100
                        100
<210> SEQ ID NO 17
<211> LENGTH: 104
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 17
Arg Thr Val Ala Ala Pro Ser Val Phe Ile Phe Pro Pro Ser Asp Glu
1 5 10 15
1 5 10 15
Gln Leu Lys Ser Gly Thr Ala Ser Val Val Cys Leu Leu Asn Asn Phe
20 25 30
                        20 25 30
Tyr Pro Arg Glu Ala Lys Val Gln Trp Lys Val Asp Asn Ala Leu Gln
35 40 45
                35 40 45
Ser Gly Asn Ser Gln Glu Ser Val Thr Glu Gln Asp Ser Lys Asp Ser
50 55 60
        50 55 60
Thr Tyr Ser Leu Ser Ser Thr Leu Thr Leu Ser Lys Ala Asp Tyr Glu
65 70 75 80
Lys His Lys Val Tyr Ala Cys Glu Val Thr His Gln Gly Leu Ser Ser
85 90 95
                                85 90 95
Pro Val Thr Lys Ser Phe Asn Arg
100
                        100
<210> SEQ ID NO 18
<211> LENGTH: 322
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 18
Met Arg Phe Pro Ser Ile Phe Thr Ala Val Leu Phe Ala Ala Ser Ser
1 5 10 15
1 5 10 15
Ala Leu Ala Ala Pro Val Asn Thr Thr Thr Glu Asp Glu Thr Ala Gln
20 25 30
                        20 25 30
Ile Pro Ala Glu Ala Val Ile Gly Tyr Ser Asp Leu Glu Gly Asp Phe
35 40 45
                35 40 45
Asp Val Ala Val Leu Pro Phe Ser Asn Ser Thr Asn Asn Gly Leu Leu
50 55 60
        50 55 60
Phe Ile Asn Thr Thr Ile Ala Ser Ile Ala Ala Lys Glu Glu Gly Val
65 70 75 80
Ser Leu Glu Lys Arg Gly Asp His His His His His His Val Val Asp
85 90 95
                                85 90 95
Lys Thr His Thr Cys Pro Pro Cys Pro Ala Pro Glu Leu Leu Gly Gly
100 105 110
                        100 105 110
Pro Ser Val Phe Leu Phe Pro Pro Lys Pro Lys Asp Thr Leu Met Ile
115 120 125
                115 120 125
Ser Arg Thr Pro Glu Val Thr Cys Val Val Val Asp Val Ser His Glu
Ser Arg Thr Pro Glu Val Thr Cys Val Val Val Asp Val Ser His Glu
        130 135 140
Asp Pro Glu Val Lys Phe Asn Trp Tyr Val Asp Gly Val Glu Val His
145 150 155 160
Asn Ala Lys Thr Lys Pro Arg Glu Glu Gln Tyr Asn Ser Thr Tyr Arg
165 170 175
                                165 170 175
Val Val Ser Val Leu Thr Val Leu His Gln Asp Trp Leu Asn Gly Lys
180 185 190
                        180 185 190
Glu Tyr Lys Cys Lys Val Ser Asn Lys Ala Leu Pro Ala Pro Ile Glu
195 200 205
                195 200 205
Lys Thr Ile Ser Lys Ala Lys Gly Gln Pro Arg Glu Pro Gln Val Tyr
210 215 220
        210 215 220
Thr Leu Pro Pro Ser Arg Asp Glu Leu Thr Lys Asn Gln Val Ser Leu
225 230 235 240
Thr Cys Leu Val Lys Gly Phe Tyr Pro Ser Asp Ile Ala Val Glu Trp
245 250 255
                                245 250 255
Glu Ser Asn Gly Gln Pro Glu Asn Asn Tyr Lys Thr Thr Pro Pro Val
260 265 270
                        260 265 270
Leu Asp Ser Asp Gly Ser Phe Phe Leu Tyr Ser Lys Leu Thr Val Asp
275 280 285
                275 280 285
Lys Ser Arg Trp Gln Gln Gly Asn Val Phe Ser Cys Ser Val Met His
290 295 300
        290 295 300
Glu Ala Leu His Asn His Tyr Thr Gln Lys Ser Leu Ser Leu Ser Pro
305 310 315 320
Gly Lys
<210> SEQ ID NO 19
<211> LENGTH: 983
<212> TYPE: DNA
<213> ORGANISM: Artificial Sequence
<220> FEATURE:
<223> OTHER INFORMATION: Description of Artificial Sequence: Synthetic
            nucleotide sequence
<220> FEATURE:
<221> NAME/KEY: CDS
<222> LOCATION: (1)..(966)
<400> SEQUENCE: 19
atg aga ttt cct tca att ttt act gca gtt tta ttc gca gca tcc tcc 48
atg aga ttt cct tca att ttt act gca gtt tta ttc gca gca tcc tcc 48
1 5 10 15
1 5 10 15
gca tta gct gct cca gtc aac act aca aca gaa gat gaa acg gca caa 96
gca tta gct gct cca gtc aac act aca aca gaa gat gaa acg gca caa 96
20 25 30
                        20 25 30
att ccg gct gaa gct gtc atc ggt tac tca gat tta gaa ggg gat ttc 144
att ccg gct gaa gct gtc atc ggt tac tca gat tta gaa ggg gat ttc 144
35 40 45
                35 40 45
gat gtt gct gtt ttg cca ttt tcc aac agc aca aat aac ggg tta ttg 192
gat gtt gct gtt ttg cca ttt tcc aac agc aca aat aac ggg tta ttg 192
50 55 60
        50 55 60
ttt ata aat act act att gcc agc att gct gct aaa gaa gaa ggg gta 240
ttt ata aat act act att gcc agc att gct gct aaa gaa gaa ggg gta 240
65 70 75 80
tct ctc gag aaa aga ggt gac cac cat cac cac cat cat gtc gtc gac 288
tct ctc gag aaa aga ggt gac cac cat cac cac cat cat gtc gtc gac 288
85 90 95
                                85 90 95
aaa act cac aca tgc cca ccg tgc cca gca cct gaa ctc ctg ggg gga 336
aaa act cac aca tgc cca ccg tgc cca gca cct gaa ctc ctg ggg gga 336
100 105 110
                        100 105 110
ccg tca gtc ttc ctc ttc ccc cca aaa ccc aag gac acc ctc atg atc 384
ccg tca gtc ttc ctc ttc ccc cca aaa ccc aag gac acc ctc atg atc 384
115 120 125
                115 120 125
tcc cgg acc cct gag gtc aca tgc gtg gtg gtg gac gtg agc cac gaa 432
tcc cgg acc cct gag gtc aca tgc gtg gtg gtg gac gtg agc cac gaa 432
130 135 140
        130 135 140
gac cct gag gtc aag ttc aac tgg tac gtg gac ggc gtg gag gtg cat 480
gac cct gag gtc aag ttc aac tgg tac gtg gac ggc gtg gag gtg cat 480
145 150 155 160
aat gcc aag aca aag ccg cgg gag gag cag tac aac agc acg tac cgt 528
aat gcc aag aca aag ccg cgg gag gag cag tac aac agc acg tac cgt 528
165 170 175
                                165 170 175
gtg gtc agc gtc ctc acc gtc ctg cac cag gac tgg ctg aat ggc aag 576
gtg gtc agc gtc ctc acc gtc ctg cac cag gac tgg ctg aat ggc aag 576
180 185 190
                        180 185 190
gag tac aag tgc aag gtc tcc aac aaa gcc ctc cca gcc ccc atc gag 624
gag tac aag tgc aag gtc tcc aac aaa gcc ctc cca gcc ccc atc gag 624
195 200 205
                195 200 205
aaa acc atc tcc aaa gcc aaa ggg cag ccc cga gaa cca cag gtg tac 672
aaa acc atc tcc aaa gcc aaa ggg cag ccc cga gaa cca cag gtg tac 672
210 215 220
        210 215 220
acc ctg ccc cca tcc cgg gat gag ctg acc aag aac cag gtc agc ctg 720
acc ctg ccc cca tcc cgg gat gag ctg acc aag aac cag gtc agc ctg 720
225 230 235 240
acc tgc ctg gtc aaa ggc ttc tat ccc agc gac atc gcc gtg gag tgg 768
acc tgc ctg gtc aaa ggc ttc tat ccc agc gac atc gcc gtg gag tgg 768
245 250 255
                                245 250 255
gag agc aat ggg cag ccg gag aac aac tac aag acc acg cct ccc gtg 816
gag agc aat ggg cag ccg gag aac aac tac aag acc acg cct ccc gtg 816
260 265 270
                        260 265 270
ttg gac tcc gac ggc tcc ttc ttc ctc tac agc aag ctc acc gtg gac 864
ttg gac tcc gac ggc tcc ttc ttc ctc tac agc aag ctc acc gtg gac 864
275 280 285
                275 280 285
aag agc agg tgg cag cag ggg aac gtc ttc tca tgc tcc gtg atg cat 912
aag agc agg tgg cag cag ggg aac gtc ttc tca tgc tcc gtg atg cat 912
290 295 300
        290 295 300
gag gct ctg cac aac cac tac acg cag aag agc ctc tcc ctg tct ccc 960
gag gct ctg cac aac cac tac acg cag aag agc ctc tcc ctg tct ccc 960
305 310 315 320
ggg aaa taatgagcgg ccgcgaa 983
ggg aaa taatgagcgg ccgcgaa 983
<210> SEQ ID NO 20
<211> LENGTH: 468
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 20
Met Asp Trp Thr Trp Arg Val Phe Cys Leu Leu Ala Val Ala Pro Gly
1 5 10 15
1 5 10 15
Ala His Ser Glu Val Gln Leu Val Glu Ser Gly Gly Gly Leu Val Lys
20 25 30
                        20 25 30
Pro Gly Gly Ser Leu Arg Leu Ser Cys Ala Ala Ser Gly Phe Thr Phe
Pro Gly Gly Ser Leu Arg Leu Ser Cys Ala Ala Ser Gly Phe Thr Phe
                35 40 45
Ser Asp Tyr Tyr Met Tyr Trp Phe Arg Gln Ala Pro Gly Lys Gly Leu
50 55 60
        50 55 60
Glu Trp Val Ala Thr Ile Ser Asp Gly Gly Ser Tyr Thr Tyr Tyr Pro
65 70 75 80
Asp Ser Val Lys Gly Arg Phe Thr Ile Ser Arg Asp Asn Ala Lys Asn
85 90 95
                                85 90 95
Ser Leu Tyr Leu Gln Met Ser Ser Leu Arg Ala Glu Asp Thr Ala Val
100 105 110
                        100 105 110
Tyr Tyr Cys Ala Arg Glu Glu Asn Gly Asn Phe Tyr Tyr Phe Asp Tyr
115 120 125
                115 120 125
Trp Gly Gln Gly Thr Thr Val Thr Val Ser Ser Ala Ser Thr Lys Gly
130 135 140
        130 135 140
Pro Ser Val Phe Pro Leu Ala Pro Ser Ser Lys Ser Thr Ser Gly Gly
145 150 155 160
Thr Ala Ala Leu Gly Cys Leu Val Lys Asp Tyr Phe Pro Glu Pro Val
165 170 175
                                165 170 175
Thr Val Ser Trp Asn Ser Gly Ala Leu Thr Ser Gly Val His Thr Phe
180 185 190
                        180 185 190
Pro Ala Val Leu Gln Ser Ser Gly Leu Tyr Ser Leu Ser Ser Val Val
195 200 205
                195 200 205
Thr Val Pro Ser Ser Ser Leu Gly Thr Gln Thr Tyr Ile Cys Asn Val
210 215 220
        210 215 220
Asn His Lys Pro Ser Asn Thr Lys Val Asp Lys Lys Val Glu Pro Lys
225 230 235 240
Ser Cys Asp Lys Thr His Thr Cys Pro Pro Cys Pro Ala Pro Glu Leu
245 250 255
                                245 250 255
Leu Gly Gly Pro Ser Val Phe Leu Phe Pro Pro Lys Pro Lys Asp Thr
260 265 270
                        260 265 270
Leu Met Ile Ser Arg Thr Pro Glu Val Thr Cys Val Val Val Asp Val
275 280 285
                275 280 285
Ser His Glu Asp Pro Glu Val Lys Phe Asn Trp Tyr Val Asp Gly Val
290 295 300
        290 295 300
Glu Val His Asn Ala Lys Thr Lys Pro Arg Glu Glu Gln Tyr Asn Ser
305 310 315 320
Thr Tyr Arg Val Val Ser Val Leu Thr Val Leu His Gln Asp Trp Leu
325 330 335
                                325 330 335
Asn Gly Lys Glu Tyr Lys Cys Lys Val Ser Asn Lys Ala Leu Pro Ala
340 345 350
                        340 345 350
Pro Ile Glu Lys Thr Ile Ser Lys Ala Lys Gly Gln Pro Arg Glu Pro
355 360 365
                355 360 365
Gln Val Tyr Thr Leu Pro Pro Ser Arg Asp Glu Leu Thr Lys Asn Gln
370 375 380
        370 375 380
Val Ser Leu Thr Cys Leu Val Lys Gly Phe Tyr Pro Ser Asp Ile Ala
385 390 395 400
Val Glu Trp Glu Ser Asn Gly Gln Pro Glu Asn Asn Tyr Lys Thr Thr
405 410 415
                                405 410 415
Pro Pro Val Leu Asp Ser Asp Gly Ser Phe Phe Leu Tyr Ser Lys Leu
420 425 430
                        420 425 430
Thr Val Asp Lys Ser Arg Trp Gln Gln Gly Asn Val Phe Ser Cys Ser
435 440 445
                435 440 445
Val Met His Glu Ala Leu His Asn His Tyr Thr Gln Lys Ser Leu Ser
450 455 460
        450 455 460
Leu Ser Pro Gly
465
<210> SEQ ID NO 21
<211> LENGTH: 1536
<212> TYPE: DNA
<213> ORGANISM: Artificial Sequence
<220> FEATURE:
<223> OTHER INFORMATION: Description of Artificial Sequence: Synthetic
            nucleotide sequence
<220> FEATURE:
<221> NAME/KEY: CDS
<222> LOCATION: (70)..(1473)
<400> SEQUENCE: 21
aagctagcgg ccgcggtcca accaccaatc tcaaagctct cgagctctag atatcgattc 60
catggatcc atg gac tgg acc tgg agg gtc ttc tgc ttg ctg gct gta gca 111
Met Asp Trp Thr Trp Arg Val Phe Cys Leu Leu Ala Val Ala
1 5 10
cca ggt gcc cac tcc gag gta caa ctg gtg gag tct ggg gga ggc tta 159
Pro Gly Ala His Ser Glu Val Gln Leu Val Glu Ser Gly Gly Gly Leu
15 20 25 30
gtg aag cct gga ggg tcc ctg agg ctc tcc tgt gca gcc tct gga ttc 207
Val Lys Pro Gly Gly Ser Leu Arg Leu Ser Cys Ala Ala Ser Gly Phe
35 40 45
act ttc agt gac tat tac atg tat tgg ttt cgc cag gcc ccg gga aag 255
Thr Phe Ser Asp Tyr Tyr Met Tyr Trp Phe Arg Gln Ala Pro Gly Lys
50 55 60
ggg ctg gag tgg gtc gca acc att agt gat ggt ggt agt tac acc tac 303
Gly Leu Glu Trp Val Ala Thr Ile Ser Asp Gly Gly Ser Tyr Thr Tyr
65 70 75
tat cca gac agt gtg aag ggg cga ttc acc atc tcc aga gac aat gcc 351
Tyr Pro Asp Ser Val Lys Gly Arg Phe Thr Ile Ser Arg Asp Asn Ala
80 85 90
aag aac agc ctc tac ctg cag atg agc agc ctg agg gct gag gac aca 399
Lys Asn Ser Leu Tyr Leu Gln Met Ser Ser Leu Arg Ala Glu Asp Thr
95 100 105 110
gct gtg tat tac tgc gca aga gag gag aat ggt aac ttt tac tac ttt 447
Ala Val Tyr Tyr Cys Ala Arg Glu Glu Asn Gly Asn Phe Tyr Tyr Phe
115 120 125
gac tac tgg ggc caa ggg acc acg gtc acc gtc tcc tca gcc tcc acc 495
Asp Tyr Trp Gly Gln Gly Thr Thr Val Thr Val Ser Ser Ala Ser Thr
130 135 140
aag ggc cca tcg gtc ttc ccc ctg gca ccc tcc tcc aag agc acc tct 543
Lys Gly Pro Ser Val Phe Pro Leu Ala Pro Ser Ser Lys Ser Thr Ser
145 150 155
ggg ggc aca gcg gcc ctg ggc tgc ctg gtc aag gac tac ttc ccc gaa 591
Gly Gly Thr Ala Ala Leu Gly Cys Leu Val Lys Asp Tyr Phe Pro Glu
160 165 170
ccg gtg acg gtg tcg tgg aac tca ggc gcc ctg acc agc ggc gtg cac 639
Pro Val Thr Val Ser Trp Asn Ser Gly Ala Leu Thr Ser Gly Val His
175 180 185 190
acc ttc ccg gct gtc cta cag tcc tca gga ctc tac tcc ctc agc agc 687
Thr Phe Pro Ala Val Leu Gln Ser Ser Gly Leu Tyr Ser Leu Ser Ser
195 200 205
gtg gtg acc gtg ccc tcc agc agc ttg ggc acc cag acc tac atc tgc 735
Val Val Thr Val Pro Ser Ser Ser Leu Gly Thr Gln Thr Tyr Ile Cys
210 215 220
aac gtg aat cac aag ccc agc aac acc aag gtg gac aag aaa gtt gag 783
Asn Val Asn His Lys Pro Ser Asn Thr Lys Val Asp Lys Lys Val Glu
225 230 235
ccc aaa tct tgt gac aag act cac aca tgc cca ccg tgc cca gca cct 831
Pro Lys Ser Cys Asp Lys Thr His Thr Cys Pro Pro Cys Pro Ala Pro
240 245 250
gaa ctc ctg ggg gga ccg tca gtc ttc ctc ttc ccc cca aaa ccc aag 879
Glu Leu Leu Gly Gly Pro Ser Val Phe Leu Phe Pro Pro Lys Pro Lys
255 260 265 270
gac acc ctc atg atc tcc cgg acc cct gag gtc aca tgc gtg gtg gtg 927
Asp Thr Leu Met Ile Ser Arg Thr Pro Glu Val Thr Cys Val Val Val
275 280 285
gac gtg agc cac gaa gac cct gag gtc aag ttc aac tgg tac gtg gac 975
Asp Val Ser His Glu Asp Pro Glu Val Lys Phe Asn Trp Tyr Val Asp
290 295 300
ggc gtg gag gtg cat aat gcc aag aca aag ccg cgg gag gag cag tac 1023
Gly Val Glu Val His Asn Ala Lys Thr Lys Pro Arg Glu Glu Gln Tyr
305 310 315
aac agc acg tac cgt gtg gtc agc gtc ctc acc gtc ctg cac cag gac 1071
Asn Ser Thr Tyr Arg Val Val Ser Val Leu Thr Val Leu His Gln Asp
320 325 330
tgg ctg aat ggc aag gag tac aag tgc aag gtc tcc aac aaa gcc ctc 1119
Trp Leu Asn Gly Lys Glu Tyr Lys Cys Lys Val Ser Asn Lys Ala Leu
335 340 345 350
cca gcc ccc atc gag aaa acc atc tcc aaa gcc aaa ggg cag ccc cga 1167
Pro Ala Pro Ile Glu Lys Thr Ile Ser Lys Ala Lys Gly Gln Pro Arg
355 360 365
gaa cca cag gtg tac acc ctg ccc cca tcc cgg gat gag ctg acc aag 1215
Glu Pro Gln Val Tyr Thr Leu Pro Pro Ser Arg Asp Glu Leu Thr Lys
370 375 380
aac cag gtc agc ctg acc tgc ctg gtc aaa ggc ttc tat ccc agc gac 1263
Asn Gln Val Ser Leu Thr Cys Leu Val Lys Gly Phe Tyr Pro Ser Asp
385 390 395
atc gcc gtg gag tgg gag agc aat ggg cag ccg gag aac aac tac aag 1311
Ile Ala Val Glu Trp Glu Ser Asn Gly Gln Pro Glu Asn Asn Tyr Lys
400 405 410
acc acg cct ccc gtg ttg gac tcc gac ggc tcc ttc ttc ctc tac agc 1359
Thr Thr Pro Pro Val Leu Asp Ser Asp Gly Ser Phe Phe Leu Tyr Ser
415 420 425 430
aag ctc acc gtg gac aag agc agg tgg cag cag ggg aac gtc ttc tca 1407
Lys Leu Thr Val Asp Lys Ser Arg Trp Gln Gln Gly Asn Val Phe Ser
435 440 445
tgc tcc gtg atg cat gag gct ctg cac aac cac tac acg cag aag agc 1455
Cys Ser Val Met His Glu Ala Leu His Asn His Tyr Thr Gln Lys Ser
450 455 460
ctc tcc ctg tct ccc ggt tgaggatcct cacatcccaa tccgcggccg 1503
Leu Ser Leu Ser Pro Gly
465
caattcgtaa tcatggtcat agctgtttcc tgt 1536
<210> SEQ ID NO 22
<211> LENGTH: 467
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 22
Met Gly Trp Ser Leu Ile Leu Leu Phe Leu Val Ala Val Ala Thr Arg
1 5 10 15
1 5 10 15
Val Leu Ser Gln Val Gln Leu Val Glu Ser Gly Ala Glu Val Lys Lys
20 25 30
                        20 25 30
Pro Gly Ala Ser Val Lys Val Ser Cys Lys Ala Ser Gly Tyr Thr Phe
35 40 45
                35 40 45
Thr Ser Tyr Trp Ile His Trp Val Arg Gln Ala Pro Gly Gln Gly Leu
50 55 60
        50 55 60
Glu Trp Ile Gly Glu Asn Asp Pro Ser Asn Gly Arg Thr Asn Tyr Asn
65 70 75 80
Glu Lys Phe Lys Asn Arg Val Thr Leu Thr Val Asp Thr Ser Ile Ser
85 90 95
                                85 90 95
Thr Ala Tyr Met His Leu Ser Ser Leu Arg Ser Asp Asp Thr Ala Val
100 105 110
                        100 105 110
Tyr Tyr Cys Ala Arg Gly Pro Asn Tyr Phe Tyr Ser Met Asp Tyr Trp
115 120 125
                115 120 125
Gly Gln Gly Thr Met Val Thr Val Ser Ser Ala Ser Thr Lys Gly Pro
130 135 140
        130 135 140
Ser Val Phe Pro Leu Ala Pro Ser Ser Lys Ser Thr Ser Gly Gly Thr
145 150 155 160
Ala Ala Leu Gly Cys Leu Val Lys Asp Tyr Phe Pro Glu Pro Val Thr
165 170 175
                                165 170 175
Val Ser Trp Asn Ser Gly Ala Leu Thr Ser Gly Val His Thr Phe Pro
180 185 190
                        180 185 190
Ala Val Leu Gln Ser Ser Gly Leu Tyr Ser Leu Ser Ser Val Val Thr
195 200 205
                195 200 205
Val Pro Ser Ser Ser Leu Gly Thr Gln Thr Tyr Ile Cys Asn Val Asn
210 215 220
        210 215 220
His Lys Pro Ser Asn Thr Lys Val Asp Lys Lys Val Glu Pro Lys Ser
225 230 235 240
Cys Asp Lys Thr His Thr Cys Pro Pro Cys Pro Ala Pro Glu Leu Leu
245 250 255
                                245 250 255
Gly Gly Pro Ser Val Phe Leu Phe Pro Pro Lys Pro Lys Asp Thr Leu
260 265 270
                        260 265 270
Met Ile Ser Arg Thr Pro Glu Val Thr Cys Val Val Val Asp Val Ser
275 280 285
                275 280 285
His Glu Asp Pro Glu Val Lys Phe Asn Trp Tyr Val Asp Gly Val Glu
290 295 300
        290 295 300
Val His Asn Ala Lys Thr Lys Pro Arg Glu Glu Gln Tyr Asn Ser Thr
305 310 315 320
Tyr Arg Val Val Ser Val Leu Thr Val Leu His Gln Asp Trp Leu Asn
325 330 335
                                325 330 335
Gly Lys Glu Tyr Lys Cys Lys Val Ser Asn Lys Ala Leu Pro Ala Pro
340 345 350
                        340 345 350
Ile Glu Lys Thr Ile Ser Lys Ala Lys Gly Gln Pro Arg Glu Pro Gln
355 360 365
                355 360 365
Val Tyr Thr Leu Pro Pro Ser Arg Asp Glu Leu Thr Lys Asn Gln Val
370 375 380
        370 375 380
Ser Leu Thr Cys Leu Val Lys Gly Phe Tyr Pro Ser Asp Ile Ala Val
385 390 395 400
Glu Trp Glu Ser Asn Gly Gln Pro Glu Asn Asn Tyr Lys Thr Thr Pro
405 410 415
                                405 410 415
Pro Val Leu Asp Ser Asp Gly Ser Phe Phe Leu Tyr Ser Lys Leu Thr
420 425 430
                        420 425 430
Val Asp Lys Ser Arg Trp Gln Gln Gly Asn Val Phe Ser Cys Ser Val
435 440 445
                435 440 445
Met His Glu Ala Leu His Asn His Tyr Thr Gln Lys Ser Leu Ser Leu
450 455 460
        450 455 460
Ser Pro Gly
465
<210> SEQ ID NO 23
<211> LENGTH: 1453
<212> TYPE: DNA
<213> ORGANISM: Artificial Sequence
<220> FEATURE:
<223> OTHER INFORMATION: Description of Artificial Sequence: Synthetic
            nucleotide sequence
<220> FEATURE:
<221> NAME/KEY: CDS
<222> LOCATION: (33)..(1433)
<400> SEQUENCE: 23
gtcaccgtcc ttgacacggg atccgcggcc gc atg ggt tgg agc ctc atc ttg 53
Met Gly Trp Ser Leu Ile Leu
1 5
ctc ttc ctt gtc gct gtt gct acg cgt gtc ctg tcc cag gtc cag ctg 101
Leu Phe Leu Val Ala Val Ala Thr Arg Val Leu Ser Gln Val Gln Leu
10 15 20
gtg gag agt ggg gct gag gtg aag aag ccc ggg gct tca gtg aag gtg 149
Val Glu Ser Gly Ala Glu Val Lys Lys Pro Gly Ala Ser Val Lys Val
25 30 35
tcc tgc aag gct tct ggc tac acc ttc acc agc tac tgg ata cac tgg 197
Ser Cys Lys Ala Ser Gly Tyr Thr Phe Thr Ser Tyr Trp Ile His Trp
40 45 50 55
gtg aga cag gcg cct gga cag ggc ctt gag tgg att gga gag aat gat 245
Val Arg Gln Ala Pro Gly Gln Gly Leu Glu Trp Ile Gly Glu Asn Asp
60 65 70
cct agc aac ggt cgt act aac tac aat gag aag ttc aag aac cgg gtc 293
Pro Ser Asn Gly Arg Thr Asn Tyr Asn Glu Lys Phe Lys Asn Arg Val
75 80 85
acc ctg act gtc gac aca tcc atc agc aca gcc tac atg cat ctc agc 341
Thr Leu Thr Val Asp Thr Ser Ile Ser Thr Ala Tyr Met His Leu Ser
90 95 100
agc ctg aga tct gac gac acc gcg gtc tat tac tgc gca agg ggc cct 389
Ser Leu Arg Ser Asp Asp Thr Ala Val Tyr Tyr Cys Ala Arg Gly Pro
105 110 115
aat tac ttc tat tct atg gac tac tgg ggt caa gga acc atg gtc acc 437
Asn Tyr Phe Tyr Ser Met Asp Tyr Trp Gly Gln Gly Thr Met Val Thr
120 125 130 135
gtc tcc tca gct agc acc aag ggc cca tcg gtc ttc ccc ctg gca ccc 485
Val Ser Ser Ala Ser Thr Lys Gly Pro Ser Val Phe Pro Leu Ala Pro
140 145 150
tcc tcc aag agc acc tct ggg ggc aca gcg gcc ctg ggc tgc ctg gtc 533
Ser Ser Lys Ser Thr Ser Gly Gly Thr Ala Ala Leu Gly Cys Leu Val
155 160 165
aag gac tac ttc ccc gaa ccg gtg acg gtg tcg tgg aac tca ggc gcc 581
Lys Asp Tyr Phe Pro Glu Pro Val Thr Val Ser Trp Asn Ser Gly Ala
170 175 180
ctg acc agc ggc gtg cac acc ttc ccg gct gtc cta cag tcc tca gga 629
Leu Thr Ser Gly Val His Thr Phe Pro Ala Val Leu Gln Ser Ser Gly
185 190 195
ctc tac tcc ctc agc agc gtg gtg acc gtg ccc tcc agc agc ttg ggc 677
Leu Tyr Ser Leu Ser Ser Val Val Thr Val Pro Ser Ser Ser Leu Gly
200 205 210 215
acc cag acc tac atc tgc aac gtg aat cac aag ccc agc aac acc aag 725
Thr Gln Thr Tyr Ile Cys Asn Val Asn His Lys Pro Ser Asn Thr Lys
220 225 230
gtg gac aag aaa gtt gag ccc aaa tct tgt gac aaa act cac aca tgc 773
Val Asp Lys Lys Val Glu Pro Lys Ser Cys Asp Lys Thr His Thr Cys
235 240 245
cca ccg tgc cca gca cct gaa ctc ctg ggg gga ccg tca gtc ttc ctc 821
Pro Pro Cys Pro Ala Pro Glu Leu Leu Gly Gly Pro Ser Val Phe Leu
250 255 260
ttc ccc cca aaa ccc aag gac acc ctc atg atc tcc cgg acc cct gag 869
Phe Pro Pro Lys Pro Lys Asp Thr Leu Met Ile Ser Arg Thr Pro Glu
265 270 275
gtc aca tgc gtg gtg gtg gac gtg agc cac gaa gac cct gag gtc aag 917
Val Thr Cys Val Val Val Asp Val Ser His Glu Asp Pro Glu Val Lys
280 285 290 295
ttc aac tgg tac gtg gac ggc gtg gag gtg cat aat gcc aag aca aag 965
Phe Asn Trp Tyr Val Asp Gly Val Glu Val His Asn Ala Lys Thr Lys
300 305 310
ccg cgg gag gag cag tac aac agc acg tac cgt gtg gtc agc gtc ctc 1013
Pro Arg Glu Glu Gln Tyr Asn Ser Thr Tyr Arg Val Val Ser Val Leu
315 320 325
acc gtc ctg cac cag gac tgg ctg aat ggc aag gag tac aag tgc aag 1061
Thr Val Leu His Gln Asp Trp Leu Asn Gly Lys Glu Tyr Lys Cys Lys
330 335 340
gtc tcc aac aaa gcc ctc cca gcc ccc atc gag aaa acc atc tcc aaa 1109
Val Ser Asn Lys Ala Leu Pro Ala Pro Ile Glu Lys Thr Ile Ser Lys
345 350 355
gcc aaa ggg cag ccc cga gaa cca cag gtg tac acc ctg ccc cca tcc 1157
Ala Lys Gly Gln Pro Arg Glu Pro Gln Val Tyr Thr Leu Pro Pro Ser
360 365 370 375
cgg gat gag ctg acc aag aac cag gtc agc ctg acc tgc ctg gtc aaa 1205
Arg Asp Glu Leu Thr Lys Asn Gln Val Ser Leu Thr Cys Leu Val Lys
380 385 390
ggc ttc tat ccc agc gac atc gcc gtg gag tgg gag agc aat ggg cag 1253
Gly Phe Tyr Pro Ser Asp Ile Ala Val Glu Trp Glu Ser Asn Gly Gln
395 400 405
ccg gag aac aac tac aag acc acg cct ccc gtg ctg gac tcc gac ggc 1301
Pro Glu Asn Asn Tyr Lys Thr Thr Pro Pro Val Leu Asp Ser Asp Gly
410 415 420
tcc ttc ttc ctc tac agc aag ctc acc gtg gac aag agc agg tgg cag 1349
Ser Phe Phe Leu Tyr Ser Lys Leu Thr Val Asp Lys Ser Arg Trp Gln
425 430 435
cag ggg aac gtc ttc tca tgc tcc gtg atg cat gag gct ctg cac aac 1397
Gln Gly Asn Val Phe Ser Cys Ser Val Met His Glu Ala Leu His Asn
440 445 450 455
cac tac acg cag aag agc ctc tcc ctg tct ccg ggt tgagcggccg 1443
His Tyr Thr Gln Lys Ser Leu Ser Leu Ser Pro Gly
460 465
cggatccctg 1453
<210> SEQ ID NO 24
<211> LENGTH: 254
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 24
Met Lys Lys Thr Ala Ile Ala Ile Ala Val Ala Leu Ala Gly Phe Ala
1 5 10 15
1 5 10 15
Thr Val Ala Gln Ala Glu Val Gln Leu Val Glu Ser Gly Gly Gly Leu
20 25 30
                        20 25 30
Val Lys Pro Gly Gly Ser Leu Arg Leu Ser Cys Ala Ala Ser Gly Phe
35 40 45
                35 40 45
Thr Phe Ser Asp Tyr Tyr Met Tyr Trp Phe Arg Gln Ala Pro Gly Lys
50 55 60
        50 55 60
Gly Leu Glu Trp Val Ala Thr Ile Ser Asp Gly Gly Ser Tyr Thr Tyr
65 70 75 80
Tyr Pro Asp Ser Val Lys Gly Arg Phe Thr Ile Ser Arg Asp Asn Ala
85 90 95
                                85 90 95
Lys Asn Ser Leu Tyr Leu Gln Met Ser Ser Leu Arg Ala Glu Asp Thr
100 105 110
                        100 105 110
Ala Val Tyr Tyr Cys Ala Arg Glu Glu Asn Gly Asn Phe Tyr Tyr Phe
115 120 125
                115 120 125
Asp Tyr Trp Gly Gln Gly Thr Thr Val Thr Val Ser Ser Ala Ser Thr
130 135 140
        130 135 140
Lys Gly Pro Ser Val Phe Pro Leu Ala Pro Ser Ser Lys Ser Thr Ser
145 150 155 160
Gly Gly Thr Ala Ala Leu Gly Cys Leu Val Lys Asp Tyr Phe Pro Glu
165 170 175
                                165 170 175
Pro Val Thr Val Ser Trp Asn Ser Gly Ala Leu Thr Ser Gly Val His
180 185 190
                        180 185 190
Thr Phe Pro Ala Val Leu Gln Ser Ser Gly Leu Tyr Ser Leu Ser Ser
195 200 205
                195 200 205
Val Val Thr Val Pro Ser Ser Ser Leu Gly Thr Gln Thr Tyr Ile Cys
210 215 220
        210 215 220
Asn Val Asn His Lys Pro Ser Asn Thr Lys Val Asp Lys Lys Val Glu
225 230 235 240
Pro Lys Ser Cys Asp Lys Thr His His His His His His His
245 250
                                245 250
<210> SEQ ID NO 25
<211> LENGTH: 833
<212> TYPE: DNA
<213> ORGANISM: Artificial Sequence
<220> FEATURE:
<223> OTHER INFORMATION: Description of Artificial Sequence: Synthetic
            nucleotide sequence
<220> FEATURE:
<221> NAME/KEY: CDS
<222> LOCATION: (57)..(818)
<400> SEQUENCE: 25
aacagctatg accatgatta cggattcact ggaactctag ataacgaggg caaaaa atg 59
Met
1
aaa aag aca gct atc gcg att gca gtg gca ctg gct ggt ttc gct acc 107
Lys Lys Thr Ala Ile Ala Ile Ala Val Ala Leu Ala Gly Phe Ala Thr
5 10 15
gtt gcg caa gca gag gta caa ctg gtg gag tct ggg gga ggc tta gtg 155
Val Ala Gln Ala Glu Val Gln Leu Val Glu Ser Gly Gly Gly Leu Val
20 25 30
aag cct gga ggg tcc ctg agg ctc tcc tgt gca gcc tct gga ttc act 203
Lys Pro Gly Gly Ser Leu Arg Leu Ser Cys Ala Ala Ser Gly Phe Thr
35 40 45
ttc agt gac tat tac atg tat tgg ttt cgc cag gcc ccg gga aag ggg 251
Phe Ser Asp Tyr Tyr Met Tyr Trp Phe Arg Gln Ala Pro Gly Lys Gly
50 55 60 65
ctg gag tgg gtc gca acc att agt gat ggt ggt agt tac acc tac tat 299
Leu Glu Trp Val Ala Thr Ile Ser Asp Gly Gly Ser Tyr Thr Tyr Tyr
70 75 80
cca gac agt gtg aag ggg cga ttc acc atc tcc aga gac aat gcc aag 347
Pro Asp Ser Val Lys Gly Arg Phe Thr Ile Ser Arg Asp Asn Ala Lys
85 90 95
aac agc ctc tac ctg cag atg agc agc ctg agg gct gag gac aca gct 395
Asn Ser Leu Tyr Leu Gln Met Ser Ser Leu Arg Ala Glu Asp Thr Ala
100 105 110
gtg tat tac tgc gcc aga gag gag aat ggt aac ttt tac tac ttt gac 443
Val Tyr Tyr Cys Ala Arg Glu Glu Asn Gly Asn Phe Tyr Tyr Phe Asp
115 120 125
tac tgg ggc caa ggg acc acg gtc acc gtc tcc tca gcc tcc acc aag 491
Tyr Trp Gly Gln Gly Thr Thr Val Thr Val Ser Ser Ala Ser Thr Lys
130 135 140 145
ggc cca tcg gtc ttc ccc ctg gca ccc tcc tcc aag agc acc tct ggg 539
Gly Pro Ser Val Phe Pro Leu Ala Pro Ser Ser Lys Ser Thr Ser Gly
150 155 160
ggc aca gcg gcc ctg ggc tgc ctg gtc aag gac tac ttc ccc gaa cct 587
Gly Thr Ala Ala Leu Gly Cys Leu Val Lys Asp Tyr Phe Pro Glu Pro
165 170 175
gtc acg gtg tcg tgg aac tca ggc gcc ctg acc agc ggc gtg cac acc 635
Val Thr Val Ser Trp Asn Ser Gly Ala Leu Thr Ser Gly Val His Thr
180 185 190
ttc ccg gct gtc cta cag tcc tca gga ctc tac tcc ctc agc agc gtg 683
Phe Pro Ala Val Leu Gln Ser Ser Gly Leu Tyr Ser Leu Ser Ser Val
195 200 205
gtg acc gtg ccc tcc agc agc ttg ggc acc cag acc tac atc tgc aac 731
Val Thr Val Pro Ser Ser Ser Leu Gly Thr Gln Thr Tyr Ile Cys Asn
210 215 220 225
gtg aat cac aag ccc agc aac acc aag gtg gac aag aaa gtt gag ccc 779
Val Asn His Lys Pro Ser Asn Thr Lys Val Asp Lys Lys Val Glu Pro
230 235 240
aaa tct tgt gac aag act cac cac cac cac cac cac cat taaccatgga 828
Lys Ser Cys Asp Lys Thr His His His His His His His
245 250
gaaaa 833
<210> SEQ ID NO 26
<211> LENGTH: 235
<212> TYPE: PRT
<213> ORGANISM: Artificial Sequence
<220> FEATURE:
<223> OTHER INFORMATION: Description of Artificial Sequence: Synthetic
            amino acid sequence
<400> SEQUENCE: 26
Val Lys Gln Ser Thr Ile Ala Leu Ala Leu Leu Pro Leu Leu Phe Thr
1 5 10 15
1 5 10 15
20 25 30
                        20 25 30
Ser Ala Ser Val Gly Asp Arg Val Thr Ile Thr Cys Lys Ala Gly Gln
35 40 45
                35 40 45
Asp Ile Lys Ser Tyr Leu Ser Trp Tyr Gln Gln Lys Pro Gly Lys Ala
50 55 60
        50 55 60
Pro Lys Leu Leu Ile Tyr Tyr Ala Thr Arg Leu Ala Asp Gly Val Pro
65 70 75 80
Ser Arg Phe Ser Gly Ser Gly Ser Gly Thr Asp Tyr Thr Leu Thr Ile
85 90 95
                                85 90 95
Ser Ser Leu Gln Pro Glu Asp Phe Ala Thr Tyr Tyr Cys Leu Gln His
100 105 110
                        100 105 110
Gly Glu Ser Pro Trp Thr Phe Gly Gly Gly Thr Lys Leu Glu Ile Lys
115 120 125
                115 120 125
Arg Thr Val Ala Ala Pro Ser Val Phe Ile Phe Pro Pro Ser Asp Glu
130 135 140
        130 135 140
Gln Leu Lys Ser Gly Thr Ala Ser Val Val Cys Leu Leu Asn Asn Phe
145 150 155 160
Tyr Pro Arg Glu Ala Lys Val Gln Trp Lys Val Asp Asn Ala Leu Gln
165 170 175
                                165 170 175
Ser Gly Asn Ser Gln Glu Ser Val Thr Glu Gln Asp Ser Lys Asp Ser
180 185 190
                        180 185 190
Thr Tyr Ser Leu Ser Ser Thr Leu Thr Leu Ser Lys Ala Asp Tyr Glu
195 200 205
                195 200 205
Lys His Lys Val Tyr Ala Cys Glu Val Thr His Gln Gly Leu Ser Ser
210 215 220
        210 215 220
Pro Val Thr Lys Ser Phe Asn Arg Gly Glu Cys
225 230 235
<210> SEQ ID NO 27
<211> LENGTH: 784
<212> TYPE: DNA
<213> ORGANISM: Artificial Sequence
<220> FEATURE:
<223> OTHER INFORMATION: Description of Artificial Sequence: Synthetic
            nucleotide sequence
<220> FEATURE:
<221> NAME/KEY: CDS
<222> LOCATION: (16)..(720)
<400> SEQUENCE: 27
catggagaaa ataaa gtg aaa caa agc act att gca ctg gca ctc tta ccg 51
Val Lys Gln Ser Thr Ile Ala Leu Ala Leu Leu Pro
1 5 10
tta ctg ttt aca ccg gtg aca aaa gcc gat atc cag atg acc cag tct 99
Leu Leu Phe Thr Pro Val Thr Lys Ala Asp Ile Gln Met Thr Gln Ser
15 20 25
cca tca tcc ttg tct gca tcg gtg gga gac agg gtc act atc act tgc 147
Pro Ser Ser Leu Ser Ala Ser Val Gly Asp Arg Val Thr Ile Thr Cys
30 35 40
aag gcg ggt cag gac att aaa agc tat tta agc tgg tac cag cag aaa 195
Lys Ala Gly Gln Asp Ile Lys Ser Tyr Leu Ser Trp Tyr Gln Gln Lys
45 50 55 60
cca ggg aaa gcg cct aag ctt ctg atc tat tat gca aca agg ttg gca 243
Pro Gly Lys Ala Pro Lys Leu Leu Ile Tyr Tyr Ala Thr Arg Leu Ala
65 70 75
gat ggg gtc cca tca aga ttc agt ggc agt gga tct ggt aca gat tat 291
Asp Gly Val Pro Ser Arg Phe Ser Gly Ser Gly Ser Gly Thr Asp Tyr
80 85 90
act cta acc atc agc agc ctg cag cct gag gat ttc gca act tat tac 339
Thr Leu Thr Ile Ser Ser Leu Gln Pro Glu Asp Phe Ala Thr Tyr Tyr
95 100 105
tgt cta cag cat ggt gag agc ccg tgg acg ttc ggt gga ggc acc aag 387
Cys Leu Gln His Gly Glu Ser Pro Trp Thr Phe Gly Gly Gly Thr Lys
110 115 120
ctg gag atc aaa cgt acg gtg gct gca cca tct gtc ttc atc ttc ccg 435
Leu Glu Ile Lys Arg Thr Val Ala Ala Pro Ser Val Phe Ile Phe Pro
125 130 135 140
cca tcc gat gag cag ttg aaa tct gga act gcc tct gtt gtg tgc ctg 483
Pro Ser Asp Glu Gln Leu Lys Ser Gly Thr Ala Ser Val Val Cys Leu
145 150 155
ctg aat aac ttc tat ccc aga gag gcc aaa gta cag tgg aag gtg gat 531
Leu Asn Asn Phe Tyr Pro Arg Glu Ala Lys Val Gln Trp Lys Val Asp
160 165 170
aac gcc ctc caa tcg ggt aac tcc cag gag agt gtc aca gag cag gac 579
Asn Ala Leu Gln Ser Gly Asn Ser Gln Glu Ser Val Thr Glu Gln Asp
175 180 185
agc aag gac agc acc tac agc ctc agc agc acc ctg acg ctg agc aaa 627
Ser Lys Asp Ser Thr Tyr Ser Leu Ser Ser Thr Leu Thr Leu Ser Lys
190 195 200
gca gac tac gag aaa cac aaa gtc tac gcc tgc gaa gtc acc cat cag 675
Ala Asp Tyr Glu Lys His Lys Val Tyr Ala Cys Glu Val Thr His Gln
205 210 215 220
ggc ctg agc tcg ccc gtc aca aag agc ttc aac agg gga gag tgt 720
Gly Leu Ser Ser Pro Val Thr Lys Ser Phe Asn Arg Gly Glu Cys
225 230 235
tagggatcca gacatgataa gctttctaga acaaaaactc atctcagaag aggatctgaa 780
tagc 784
<210> SEQ ID NO 28
<211> LENGTH: 40
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 28
gaccaagaac cactcgagta gcgtcctacc cccgtcccac 40
<210> SEQ ID NO 29
<211> LENGTH: 40
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 29
gtgggacggg ggtaggacgc tactcgagtg gttcttggtc 40
<210> SEQ ID NO 30
<211> LENGTH: 45
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 30
cggaaactgg tccgttcagt ccgactggac cgtgaaccag tcgag 45
<210> SEQ ID NO 31
<211> LENGTH: 45
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 31
ctcgactggt tcacggtcca gtcggactga acggaccagt ttccg 45
<210> SEQ ID NO 32
<211> LENGTH: 39
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 32
gcaccagaac atcaacgtga gcccgacggg taacgagag 39
<210> SEQ ID NO 33
<211> LENGTH: 39
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 33
ctctcgttac ccgtcgggct cacgttgatg ttctggtgc 39
<210> SEQ ID NO 34
<211> LENGTH: 34
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 34
ggggacgacg gtggacgtga acaggtgcca ctcg 34
<210> SEQ ID NO 35
<211> LENGTH: 34
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 35
cgagtggcac ctgttcacgt ccaccgtcgt cccc 34
<210> SEQ ID NO 36
<211> LENGTH: 35
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 36
cgccggcgag taatcgtagg ccctctgtcc ctctc 35
<210> SEQ ID NO 37
<211> LENGTH: 35
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 37
cgccggcgag taatcgtagg ccctctgtcc ctctc 35
<210> SEQ ID NO 38
<211> LENGTH: 42
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 38
cgggggtctc cacgagaacg ttctccctcg gtcccccttc tg 42
<210> SEQ ID NO 39
<211> LENGTH: 42
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 39
cagaaggggg accgagggag aacgttctcg tggagacccc cg 42
<210> SEQ ID NO 40
<211> LENGTH: 38
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 40
cgtcgggtcc cggcgccacg tgggtctcca cgagaacc 38
<210> SEQ ID NO 41
<211> LENGTH: 38
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 41
gcagcccagg gccgcggtgc acccagaggt gctcttgg 38
<210> SEQ ID NO 42
<211> LENGTH: 38
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 42
ccacacgtgc ggcgacgtgt cccgtggact caaggtgc 38
<210> SEQ ID NO 43
<211> LENGTH: 38
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 43
gcaccttgag tccacgggac acgtcgccgc acgtgtgg 38
<210> SEQ ID NO 44
<211> LENGTH: 33
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 44
gacccacggg ttcgacgtcc tcccgtgcca gtg 33
<210> SEQ ID NO 45
<211> LENGTH: 33
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 45
cactggcacg ggaggacgtc gaacccgtgg gtc 33
<210> SEQ ID NO 46
<211> LENGTH: 36
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 46
ggtctaaagt cgacgagcgt cctaccgccc ttctac 36
<210> SEQ ID NO 47
<211> LENGTH: 36
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 47
gtagaagggc ggtaggacgc tcgtcgactt tagacc 36
<210> SEQ ID NO 48
<211> LENGTH: 40
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 48
gttgtctccg tcaaggcgtg aagtcgacga gtagcctacc 40
<210> SEQ ID NO 49
<211> LENGTH: 40
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 49
ggtaggctac tcgtcgactt cacgccttga cggagacaac 40
<210> SEQ ID NO 50
<211> LENGTH: 43
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 50
ctgtgagagg accctcgttg ggctgacgtc ccgcaatagg tgg 43
<210> SEQ ID NO 51
<211> LENGTH: 43
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 51
ccacctattg cgggacgtca gcccaacgag ggtcctctca cag 43
<210> SEQ ID NO 52
<211> LENGTH: 38
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 52
cacaaagagc atcagcgtaa atgagtcgca gtcccacg 38
<210> SEQ ID NO 53
<211> LENGTH: 38
<212> TYPE: DNA
<213> ORGANISM: Artificial Sequence
<220> FEATURE:
<223> OTHER INFORMATION: Description of Artificial Sequence: Synthetic
            oligonucleotide
<400> SEQUENCE: 53
cgtgggactg cgactcattt acgctgatgc tctttgtg 38
<210> SEQ ID NO 54
<211> LENGTH: 12
<212> TYPE: PRT
<213> ORGANISM: Artificial Sequence
<220> FEATURE:
<223> OTHER INFORMATION: Description of Artificial Sequence: Synthetic
            peptide
<400> SEQUENCE: 54
Gly Cys His His His His His His Val Val Asp Lys
1 5 10
1 5 10
<210> SEQ ID NO 55
<211> LENGTH: 4
<212> TYPE: PRT
<213> ORGANISM: Artificial Sequence
<220> FEATURE:
<223> OTHER INFORMATION: Description of Artificial Sequence: Synthetic
            peptide
<400> SEQUENCE: 55
Glu Val Gln Leu
1
<210> SEQ ID NO 56
<211> LENGTH: 4
<212> TYPE: PRT
<213> ORGANISM: Artificial Sequence
<220> FEATURE:
<223> OTHER INFORMATION: Description of Artificial Sequence: Synthetic
            peptide
<400> SEQUENCE: 56
Gln Val Gln Leu
1
<210> SEQ ID NO 57
<211> LENGTH: 4
<212> TYPE: PRT
<213> ORGANISM: Artificial Sequence
<220> FEATURE:
<223> OTHER INFORMATION: Description of Artificial Sequence: Synthetic
            peptide
<400> SEQUENCE: 57
Ala Val Gln Leu
1
<210> SEQ ID NO 58
<211> LENGTH: 4
<212> TYPE: PRT
<213> ORGANISM: Artificial Sequence
<220> FEATURE:
<223> OTHER INFORMATION: Description of Artificial Sequence: Synthetic
            peptide
<400> SEQUENCE: 58
Asp Ile Gln Met
1
<210> SEQ ID NO 59
<211> LENGTH: 6
<212> TYPE: PRT
<213> ORGANISM: Artificial Sequence
<220> FEATURE:
<223> OTHER INFORMATION: Description of Artificial Sequence: Synthetic
            6xHis tag
<400> SEQUENCE: 59
His His His His His His
1 5
<210> SEQ ID NO 60
<211> LENGTH: 15
<212> TYPE: PRT
<213> ORGANISM: Artificial Sequence
<220> FEATURE:
<223> OTHER INFORMATION: Description of Artificial Sequence: Synthetic
            peptide
<400> SEQUENCE: 60
Gly Gly Gly Gly Ser Gly Gly Gly Gly Ser Gly Gly Gly Gly Ser
1 5 10 15
<210> SEQ ID NO 61
<211> LENGTH: 11
<212> TYPE: PRT
<213> ORGANISM: Artificial Sequence
<220> FEATURE:
<223> OTHER INFORMATION: Description of Artificial Sequence: Synthetic
            peptide
<400> SEQUENCE: 61
Gly Gly Gly Ser Ser Gly Gly Gly Ser Gly Gly
1 5 10
<210> SEQ ID NO 62
<211> LENGTH: 25
<212> TYPE: PRT
<213> ORGANISM: Artificial Sequence
<220> FEATURE:
<223> OTHER INFORMATION: Description of Artificial Sequence: Synthetic
            peptide
<400> SEQUENCE: 62
Gly Gly Gly Gly Ser Gly Gly Gly Gly Ser Gly Gly Gly Gly Ser Gly
1 5 10 15
Gly Gly Gly Ser Gly Gly Gly Ala Ser
                        20 25
<210> SEQ ID NO 63
<211> LENGTH: 222
<212> TYPE: PRT
<213> ORGANISM: Homo sapiens
<400> SEQUENCE: 63
Pro Cys Pro Ser Cys Pro Ala Pro Glu Phe Leu Gly Gly Pro Ser Val
1 5 10 15
Phe Leu Phe Pro Pro Lys Pro Lys Asp Thr Leu Met Ile Ser Arg Thr
                        20 25 30
Pro Glu Val Thr Cys Val Val Val Asp Val Ser Gln Glu Asp Pro Glu
                35 40 45
Val Gln Phe Asn Trp Tyr Val Asp Gly Val Glu Val His Asn Ala Lys
        50 55 60
Thr Lys Pro Arg Glu Glu Gln Phe Asn Ser Thr Tyr Arg Val Val Ser
65 70 75 80
Val Leu Thr Val Leu His Gln Asp Trp Leu Asn Gly Lys Glu Tyr Lys
                                85 90 95
Cys Lys Val Ser Asn Lys Gly Leu Pro Ser Ser Ile Glu Lys Thr Ile
                        100 105 110
Ser Lys Ala Lys Gly Gln Pro Arg Glu Pro Gln Val Tyr Thr Leu Pro
                115 120 125
Pro Ser Gln Glu Glu Met Thr Lys Asn Gln Val Ser Leu Thr Cys Leu
        130 135 140
Val Lys Gly Phe Tyr Pro Ser Asp Ile Ala Val Glu Trp Glu Ser Asn
145 150 155 160
Gly Gln Pro Glu Asn Asn Tyr Lys Thr Thr Pro Pro Val Leu Asp Ser
                                165 170 175
Asp Gly Ser Phe Phe Leu Tyr Ser Arg Leu Thr Val Asp Lys Ser Arg
                        180 185 190
Trp Gln Glu Gly Asn Val Phe Ser Cys Ser Val Met His Glu Ala Leu
                195 200 205
His Asn His Tyr Thr Gln Lys Ser Leu Ser Leu Ser Leu Gly
        210 215 220
```

What is claimed is:

1. An altered binding polypeptide comprising a first CH3 domain or portion thereof, wherein the CH3 domain comprises an engineered cysteine or thiol-containing analog thereof at amino acid position 355 or 415, according to the EU numbering index, and wherein the polypeptide is capable of expression by a host cell at a yield of at least 5 mg per liter of host culture medium.

2. An altered binding polypeptide comprising a first CH3 domain or portion thereof, wherein the CH3 domain comprises an engineered cysteine or thiol-containing analog thereof at one or more amino acid positions selected from the group consisting of 350, 355, 361, 389, 415, 441, 443, and 446b, according to the EU numbering index.

3. (canceled)

4. An altered binding polypeptide comprising a first CH3 domain or portion thereof which comprises an engineered cysteine or thiol-containing analog thereof at one or more amino acid positions selected from the group consisting of amino acid positions 350, 352, 353, 355, 358-366, 368, 370, 371, 389-391, 394-396, 398, 400-407, 409-423 441, 443, 445, 446, and 446b, according to the EU numbering index.

5. A monospecific altered binding protein comprising an altered binding polypeptide comprising an engineered cysteine or thiol-containing analog thereof at one or more amino acid positions selected from the group consisting of amino acid positions 341-441, 443, 445, and 446b, according to the EU numbering index.

6. An altered binding polypeptide wherein said altered binding polypeptide is an altered antibody variant chain comprising a first CH3 domain or portion thereof which comprises an engineered cysteine or thiol-containing analog thereof at one or more amino acid positions selected from the group consisting of 341-441, 443, 445, and 446b, according to the EU numbering index.

7. The altered binding polypeptide of any of the preceding claims which comprises at least two engineered cysteine residues or thiol-containing analogs thereof.

8. (canceled)

9. (canceled)

10. The altered binding polypeptide claims 1, 2, or 3 which comprises at least one CL domain or portion thereof wherein the CL domain comprises an engineered cysteine or thiol-containing analog thereof at one or more of amino acid positions 108-211, according to the Kabat numbering index.

11. (canceled)

12. The altered binding polypeptide of claims 1, 2, or 3, further comprising a second CH3 domain comprising an engineered cysteine or thiol-containing analog thereof.

13. (canceled)

14. (canceled)

15. (canceled)

16. (canceled)

17. An altered binding polypeptide comprising at least one of: (i) a CH1 domain or portion thereof comprising an engineered cysteine or thiol-containing analog thereof at one or more amino acid positions selected from the group consisting of amino acid positions 118-215, according to the EU numbering index, and (ii) a CL domain or portion thereof comprising an engineered cysteine or thiol-containing analog thereof at one or more amino acid positions selected from the group consisting of amino acid positions 108-211, according to the Kabat numbering index.

18. The altered binding polypeptide of claim 17, wherein the CH1 domain comprises an engineered cysteine residue or thiol-containing analog thereof at one or more amino acid positions selected from the group consisting of amino acid positions 132, 138, 164, and 191, according to the EU index.

19. The altered binding polypeptide of claim 17, wherein the CL domain comprises an engineered cysteine residue or thiol-containing analog thereof at one or more amino acid positions selected from the group consisting of 122, 127, 158, and 184, according to the Kabat numbering index.

20. The altered binding polypeptide of claim 17, wherein the altered binding polypeptide comprises an antigen binding portion of an antibody molecule.

21. The altered binding polypeptide of claim 17, wherein the antigen binding portion is a Fab fragment.

22. The altered binding polypeptide of claim 21, wherein the CH1 domain or the CL domain is fused to a scFv fragment.

23. The altered binding polypeptide of claim 17, wherein the binding polypeptide comprises at least one binding site selected from the group consisting of an antigen binding site, a ligand binding portion of a receptor, and a receptor binding portion of a ligand.

24. (canceled)

25. (canceled)

26. (canceled)

27. A nucleic acid molecule encoding an altered binding polypeptide of claim 1.

28. A vector comprising the nucleic acid molecule of claim 27.

29. A host cell comprising the vector of claim 28.

30. (canceled)

31. A modified binding protein of formula I:
Pro-(S—[Y-E_{q}]ₘ)ₙ (I)
wherein
a. Pro is an altered binding protein of any of claims 1-30;
b. S is a sulfur atom of an engineered cysteine residue or analog thereof of the altered binding protein;
c. Y is a linking moiety or a covalent bond, independently selected for each occurrence;
d. E is an independently selected effector moiety for each occurrence; and
e. q, m and n are each independently selected positive integers for each occurrence, wherein the effector moiety is an anti-cancer agent selected from the group consisting of a doxorubicin, a maytansanoid, an etoposide, a taxane, placlitaxel, fluorouracil, mitomycin, camptothecin, a vinca alkaloid, neocarzinostatin, calicheamicin, a maytansinoid, (RS)-cyclophosphamide, 6-mercatopurine, auristatin E, daunorubicin, and a derivative or analog thereof.

32. A modified binding protein of formula V:
Pro₁-(S—[Y—(S-Pro₂)_{q}]ₘ)ₙ (V)
wherein
Pro₁ and Pro₂ are independently selected from the altered binding proteins of any of claims 1-30;
S is a sulfur atom of an engineered cysteine residue or analog thereof;
Y is a linking moiety or a covalent bond, independently selected for each occurrence;
E is an independently selected effector moiety for each occurrence; and
q, m and n are each independently selected positive integers for each occurrence,
wherein q is 1, m is 1, n is 2, Pro₁ is an altered CH3-containing binding protein and Pro₂ is an altered CH1-containing binding protein or an altered CL-containing protein,
and wherein the modified binding protein has at least one binding specificity for a TNF ligand family member or a TNF receptor family member.

33. (canceled)

34. The modified binding protein of claim 32, wherein at least one CH1-containing binding protein has at least one binding specificity for TRAIL-R2.

35. The modified binding protein of claim 32 or 34, wherein at least one CH1-containing binding protein has at least one binding specificity for LTβR.
